(19) United States
(12) Patent Application Publication
(10) Pub. No.: US 2020/0368338 A1
(43) Pub. Date: Nov. 26, 2020

(54) CONJUGATE OF WT1-DERIVED PEPTIDES AND COMPOSITION COMPRISING THE SAME

(71) Applicants: SUMITOMO DAINIPPON PHARMA CO., LTD., Osaka-shi (JP); INTERNATIONAL INSTITUTE OF CANCER IMMUNOLOGY, INC., Suita-shi (JP)

(72) Inventors: Hitoshi BAN, Nishinomiya-shi (JP); Yosuke TAKANASHI, Osaka-shi (JP)

(73) Assignees: SUMITOMO DAINIPPON PHARMA CO., LTD., Osaka-shi (JP); INTERNATIONAL INSTITUTE OF CANCER IMMUNOLOGY, INC., Suita-shi (JP)

(21) Appl. No.: 16/957,963

(22) PCT Filed: Dec. 26, 2018
(86) PCT No.: PCT/JP2018/047752

§ 371 Date: Jun. 25, 2020

(30) Foreign Application Priority Data
Dec. 27, 2017 (JP) 2017-251568

(51) Int. Cl. A61K 39/00; A61K 47/64; C07K 14/47; A61P 35/00; C07K 7/08

(52) U.S. Cl. CPC A61K 39/001153; A61K 47/646; C07K 14/4748; A61K 2039/62; C07K 7/08; A61K 2039/6031; A61K 2039/572; A61P 35/00

(57) ABSTRACT

The present disclosure includes a compound of formula (1):

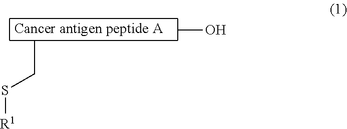

wherein cancer antigen peptide A is an MHC class I-restricted peptide consisting of 7 to 30 amino acid residues and containing at least one cysteine residue, wherein the cysteine residue of the cancer antigen peptide A binds to R¹ via a disulfide bond; and
R¹ is hydrogen, a group of formula (2), the group of formula (3), or cancer antigen peptide D,
wherein the group of formula 2 is

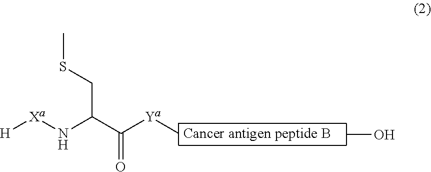

wherein X^{a} and Y^{d} independently represent a single bond or a divalent peptide group consisting of 1 to 4 amino acid residues, provided that the sum of the number of amino acid residues in X^{a} and Y^{a} is an integer of 0 to 4, and
the cancer antigen peptide B is an MHC class II-restricted peptide consisting of 9 to 30 amino acid residues, wherein the amino group of the N-terminal amino acid of the cancer antigen peptide B binds to Y^{a} in the formula (2), and the carbonyl group of the C-terminal amino acid of the cancer antigen peptide B binds to the hydroxyl group in the formula (2), and the formula (1) and the formula (2) binds via a disulfide bond,
the group of formula (3) is

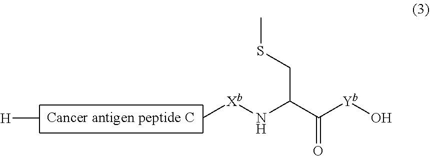

wherein X^{b} and Y^{b} independently represent a single bond or a divalent peptide group consisting of 1 to 4 amino acid residues, provided that the sum of the number of amino acid residues in X^{b} and Y^{b} is an integer of 0 to 4, and
the cancer antigen peptide C is an MHC class II-restricted peptide consisting of 9 to 30 amino acid residues, wherein the carbonyl group of the C-terminal amino acid of the cancer antigen peptide C binds to X^{b} in the formula (3), and the amino group of the N-terminal amino acid of the cancer antigen peptide C binds to the hydrogen atom in the formula (3), and the formula (1) and the formula (3) binds via a disulfide bond, and
the cancer antigen peptide D is an MHC class II-restricted peptide consisting of 9 to 30 amino acid residues and containing at least one cysteine, wherein the cysteine residue of the cancer antigen peptide D binds to R¹ via a disulfide bond;
or a pharmaceutically acceptable salt thereof.

### CROSS REFERENCE TO RELATED APPLICATIONS

This application is a national stage application of PCT/JP2018/047752, filed Dec. 26, 2018, and is based upon and claims the benefits of priority to Japanese Application No. 2017-251568, filed Dec. 27, 2017. The entire contents of all of the above applications are incorporated herein by reference.

### REFERENCE TO A SEQUENCE LISTING

In accordance with 37 CFR § 1.52(e)(5), the present specification makes reference to a Sequence Listing (submitted electronically as a .txt file named “531227US_ST25.txt”). The .txt file was generated on Jul. 1, 2020 and is 56.1 kb in size. The entire contents of the Sequence Listing are herein incorporated by reference.

### TECHNICAL FIELD

The present disclosure relates to cancer immunotherapy and includes a conjugate containing cancer antigen peptides from a cancer antigen protein WT1 and a composition comprising the conjugate.

### BACKGROUND

WT1 Gene has been isolated as a responsible gene of Wilms tumor, which is a kidney cancer in children. Leukemia and some solid cancers are known to be associated with high expression of WT1. WT1 protein is one of cancer antigen proteins of strong interest for use in cancer vaccines. (Non-patent literature 1).

Cellular immunity, especially cytotoxic T cells (cytotoxic T lymphocytes, also referred to as CTLs hereinafter) play an important role in cancer cell clearance by a living body. CTLs that attack cancer cells are derived from precursor T cells through their differentiation and proliferation, upon recognition by precursor T cells of an antigen peptide having 8 to 13 amino acid residues from a cancer antigen protein (i.e., cancer antigen peptide), complexed with an MHC class I molecule. As to WT1 protein, the following cancer antigen peptides (Patent literature 1) or a modified peptide thereof (Patent literature 2), which are bound to and presented by MHC class I, have been reported:

WT1₁₂₆₋₁₃₄ peptide:
(SEQ ID NO: 2)
RMFPNAPYL (Arg-Met-Phe-Pro-Asn-Ala-Pro-Tyr-Leu),
WT1₂₃₅₋₂₄₃ peptide:
(SEQ ID NO: 3)
CMTWNQMNL (Cys-Met-Thr-Trp-Asn-Gln-Met-Asn-Leu),
and
WT1₂₃₅₋₂₄₃ (2M→Y) peptide:
(SEQ ID NO: 4)
CYTWNQMNL (Cys-Tyr-Thr-Trp-Asn-Gln-Met-Asn-Leu).

In cancer immunotherapy, activation of helper T cells is also important to enhance functions of other T cells such as CTLs (Non-patent literatures 2 and 3). It is commonly understand that degradation of an antigen protein in lysosomes within cells produces a peptide fragment consisting of about 10 to 25 amino acid residues and a part of the peptide fragment is bound to an MHC class II molecule and presented to a TCR-CD3 complex on helper T cells to activate the cells. As to WT1 protein, the following cancer antigen peptides, which are bound to and presented by MHC class I, have been reported (Patent literatures 3 and 4):

WT1₃₅₋₅₂ peptide:
(SEQ ID NO: 237)
WAPVLDFAPPGASAYGSL (Trp-Ala-Pro-Val-Leu-Asp-Phe-
Ala-Pro-Pro-Gly-Ala-Ser-Ala-Tyr-Gly-Ser-Leu)
WT1₃₃₀₋₃₄₉ Peptide:
(SEQ ID NO: 233)
PGCNKRYFKLSHLQMHSRKHTG (Pro-Gly-Cys-Asn-Lys-Arg-
Tyr-Phe-Lys-Leu-Ser-His-Leu-Gln-Met-His-Ser-Arg-
Lys-His-Thr-Gly)

For inducing CTLs more efficiently, cocktail vaccines that comprise two different peptides being an MHC class II-restricted peptide and an MHC class I-restricted peptide have been widely used (Non-patent literatures 4-7). Cocktail vaccines comprising cancer antigen peptides from WT1 protein have also been reported (Non-patent literatures 8 and 9). However, as cocktail vaccines comprise antigen peptides composed of different amino acids and thus could show different physical properties, optimal formulations to efficiently induce CTLs corresponding to the peptides are often difficult to prepare.

Long chain peptide vaccines are also considered for efficient CTL induction, but can be difficult to prepare as proteins. Also, as long chain peptide vaccines are composed of antigen peptides to be presented on MHC class I and class II molecules, respectively, bound each other via a peptide spacer, it is difficult to control and predict its cleavage site by intracellular enzymes.

Another option for efficient CTL induction that has been reported is a conjugate of MHC class I-restricted peptides or an MHC class I-restricted peptide and an MHC class II-restricted peptide from WT1 protein via a disulfide bond (Patent literature 5). The conjugate of Patent literature 5 is characterized in that the MHC class I-restricted peptide is provided by the function of ERAP1 (Endoplasmic reticulum aminopeptiase 1), which is one of trimming enzymes that have been reported to cleave a cancer antigen peptide precursor (Non-patent literatures 10-12), and the conjugate has to include at least one MHC class I-restricted peptide to which a cysteine residue is added. Therefore, in development of WT1 conjugate vaccines being able to efficiently induce CTLs while having great physicochemical properties, further improvements have been desired in view of easy manufacturing and simple manufacturing control and wide use of the vaccines.

### CITATION LIST

### Patent Literature

Patent Literature 1: WO 00/06602
Patent Literature 2: WO 02/079253
Patent Literature 3: WO 2007/047764
Patent Literature 4: WO 2010/123065
Patent Literature 5: WO 2014/157692

### Non Patent Literature

Non Patent Literature 1: Clin Cancer Res, 2009; 15(17); 5323-37
Non Patent Literature 2: Cancer Res, 2002; 62(22); 6438-6441
Non Patent Literature 3: J Immunother, 2001; 24(3); 195-204
Non Patent Literature 4: Cancer Journal, 2011; 17(5); 343-350
Non Patent Literature 5: Cancer Sci. 2013; 104(1); 15-21
Non Patent Literature 6: Adv Immunol. 2012; 114; 51-76
Non Patent Literature 7: Recemt Pat Inflamm Allergy Drug Discov. 2015; 9(1); 38-45
Non Patent Literature 8: Blood. 2010; 116(2); 171-9
Non Patent Literature 9: Cancer Immunol Immunother. 2010; 59(10); 1467-79
Non Patent Literature 10: Proceedings of the National Academy of Sciences of United States of America, 2005; 102(47); 17107-17112
Non Patent Literature 11: The Journal of Immunology, 2009; 183; 5526-5536
Non Patent Literature 12: The Journal of Immunology, 2010; 184; 4725-4732

### SUMMARY

### Technical Problem

An object of the present disclosure is to provide a conjugate of an MHC class I-restricted peptide and an MHC class II-restricted peptide each from WT1 protein that is able to induce CTLs efficiently, and a composition comprising the conjugate that can be used as a cocktail vaccine.

The inventors have conducted intensive studies to overcome the above difficulties and found that a cysteine residue within an MHC class I-restricted peptide containing at least one cysteine residue and a cysteine residue within or added to an MHC class II-restricted peptide can be bound via a disulfide bond. Then, the inventors have provided a conjugate that is able to efficiently induce CTLs and has a favorable physicochemical stability while enabling easy manufacturing and simple manufacturing control, as it is not required to add a cysteine residue to an MHC class I-restricted peptide. Further, the conjugate can be used widely. In addition, the inventors have found that a cocktail vaccine comprising the conjugate and an MHC class I-restricted peptide monomer induces CTLs much more efficiently. Accordingly, the inventors have arrived at the invention.

### Solution to Problem

(1) First Aspect

1. A compound of formula (1):

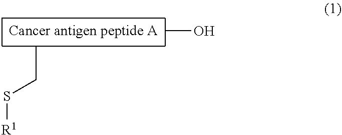

wherein cancer antigen peptide A is an MHC class I-restricted peptide consisting of 7 to 30 amino acid residues and containing at least one cysteine residue, wherein the cysteine residue of the cancer antigen peptide A binds to R¹ via a disulfide bond; and
R¹ is hydrogen, a group of formula (2), the group of formula (3), or cancer antigen peptide D, wherein the group of formula (2) is

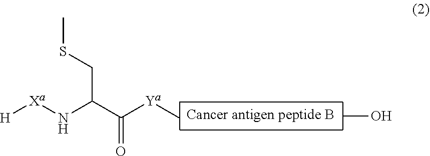

wherein X^{a} and Y^{a} independently represent a single bond or a divalent peptide group consisting of 1 to 4 amino acid residues, provided that the sum of the number of amino acid residues in X^{a} and Y^{a} is an integer of 0 to 4, and
the cancer antigen peptide B is an MHC class II-restricted peptide consisting of 9 to 30 amino acid residues, wherein the amino group of the N-terminal amino acid of the cancer antigen peptide B binds to Y^{a} in the formula (2), and the carbonyl group of the C-terminal amino acid of the cancer antigen peptide B binds to the hydroxyl group in the formula (2), and the formula (1) and the formula (2) binds via a disulfide bond,
the group of formula (3) is

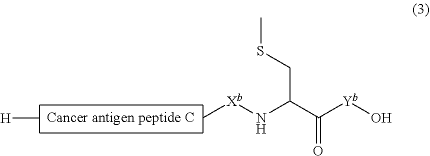

wherein X^{b} and Y^{b} independently represent a single bond or a divalent peptide group consisting of 1 to 4 amino acid residues, provided that the sum of the number of amino acid residues in X^{b} and Y^{b} is an integer of 0 to 4, and
the cancer antigen peptide C is an MHC class II-restricted peptide consisting of 9 to 30 amino acid residues, wherein the carbonyl group of the C-terminal amino acid of the cancer antigen peptide C binds to X^{b} in the formula (3), and the amino group of the N-terminal amino acid of the cancer antigen peptide C binds to the hydrogen atom in the formula (3), and the formula (1) and the formula (3) binds via a disulfide bond, and
the cancer antigen peptide D is an MHC class II-restricted peptide consisting of 9 to 30 amino acid residues and containing at least one cysteine, wherein the cysteine residue of the cancer antigen peptide D binds to R¹ via a disulfide bond;
or a pharmaceutically acceptable salt thereof.

2. The compound or pharmaceutically acceptable salt thereof of item 1, wherein the cancer antigen peptide A is a peptide consisting of 7 to 15 amino acid residues and restricted to HLA-A, HLA-B, or HLA-Cw.

3. The compound or pharmaceutically acceptable salt thereof of item 1 or 2, wherein the cancer antigen peptide A is a peptide consisting of 7 to 15 amino acid residues and restricted to at least one HLA type selected from the group consisting of HLA-A1, HLA-A2, HLA-A3, HLA-A11, HLA-A24, HLA-A28, HLA-A29, HLA-A31, HLA-A33, HLA-A34, HLA-A68.1, HLA-A1101, HLA-A0201, HLA-A0205, HLA-A3101, HLA-A3302, HLA-B7, HLA-B8, HLA-B13, HLA-B14, HLA-B35, HLA-B40, HLA-B60, HLA-B61, HLA-B62, HLA-B2702, HLA-B2705, HLA-B3501, HLA-B3701, HLA-B3801, HLA-B3901, HLA-B3902, HLA-B4403, HLA-B5101, HLA-B5102, HLA-B5201, HLA-B5801, HLA-Cw2, HLA-Cw3, HLA-Cw6, HLA-Cw7, HLA-Cw8, HLA-Cw16, HLA-Cw0301, HLA-Cw0401, HLA-Cw0602, and HLA-Cw0702.

4. The compound or pharmaceutically acceptable salt thereof of any one of items 1-3, wherein the cancer antigen peptide A is an MHC class I-restricted peptide from a cancer antigen protein selected from the group consisting of WT1, MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-3, GAGE-4, GAGE-5, GAGE-6, GAGE-7B, GAGE-8, NA88-A, NY-ESO-1, NY-ESO-1a, MART-1/Melan-A, MC1R, Gp100, PSA, PSM, Tyrosinase, Proteinase 3, TRP-1, TRP-2, ART-4, CAMEL, CEA, Ep-CAM, Cyp-B, Her2/neu, VEGFR, hTERT, hTRT, iCE, MUC1, MUC2, PRAME, P15, RU1, RU2, SART-1, SART-2, SART-3, AFP, β-Catenin, Caspase-8, CDK-4, ELF2, GnT-V, G250, HSP70-2M, HST-2, KIAA0205, MUM-1, MUM-2, MUM-3, Myosin, RAGE, SART-2, TRP-2, 707-AP, Survivin, Livin and SYT-SSX.

5. The compound or pharmaceutically acceptable salt thereof of any one of items 1-4, wherein the cancer antigen peptide A is an MHC class I-restricted WT1 peptide consisting of 7 to 12 amino acid residues.

6. The compound or pharmaceutically acceptable salt thereof of any one of items 1-5, wherein the cancer antigen peptide A is an MHC class I-restricted WT1 peptide consisting of 8 to 10 amino acid residues.

7. The compound or pharmaceutically acceptable salt thereof of any one of items 1-6, wherein the cancer antigen peptide A is an MHC class I-restricted WT1 peptide consisting of 9 amino acid residues.

8. The compound or pharmaceutically acceptable salt thereof of item 7, wherein the cancer antigen peptide A is a peptide comprising the acid sequence:

(SEQ ID NO: 3)
CMTWNQMNL

or a peptide comprising an amino acid sequence that differs from the amino acid sequence of SEQ ID NO: 3 by alteration of one or several amino acid residues and having an ability to induce CTLs.

9. The compound or pharmaceutically acceptable salt thereof of item 8, wherein the cancer antigen peptide A is a peptide comprising an amino acid sequence selected from the group consisting of

(SEQ ID NO: 3)
CMTWNQMNL
and
(SEQ ID NO: 4)
CYTWNQMNL.

10. The compound or pharmaceutically acceptable salt thereof of any one of items 1-9, wherein R¹ is the group of formula (2).

11. The compound or pharmaceutically acceptable salt thereof of any one of items 1-10, wherein X^{a} is a divalent peptide group consisting of two amino acid residues, and Y^{a} is a single bond; X^{a} and Y^{a} are independently a divalent peptide group consisting of one amino acid residue; X^{a} is a single bond, and Y^{a} is a divalent peptide group consisting of two amino acid residues; X^{a} is a divalent peptide group consisting of one amino acid residue, and Y^{a} is a single bond; X^{a} is a single bond, and Y^{a} is a divalent peptide group consisting of one amino acid residue; or X^{a} and Y^{a} are single bonds.

12. The compound or pharmaceutically acceptable salt thereof of item 11, wherein X^{a} is a single bond and Y^{a} is a single bond or a divalent peptide group consisting of one amino acid residue.

13. The compound or pharmaceutically acceptable salt thereof of item 11, wherein X is a single bond or a divalent peptide group consisting of one amino acid residue and Y^{a} is a single bond.

14. The compound or pharmaceutically acceptable salt thereof of any one of items 11-13, wherein X^{a} and Y^{a} are single bonds.

15. The compound or pharmaceutically acceptable salt thereof of any one of items 1-14, wherein the cancer antigen peptide B is an MHC class II-restricted WT1 peptide consisting of 9 to 30 amino acid residues.

16. The compound or pharmaceutically acceptable salt thereof of item 15, wherein the cancer antigen peptide B is an MHC class II-restricted WT1 peptide consisting of 10 to 25 amino acid residues.

17. The compound or pharmaceutically acceptable salt thereof of any one of items 1-16, wherein the cancer antigen peptide B is a peptide comprising an amino acid sequence selected from:

(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
and
(SEQ ID NO: 248)
RYFKLSHLQMHSRKHTGE

or a peptide comprising an amino acid sequence that differs from the amino acid sequence selected from SEQ ID NOS: 217-248 by alteration of one or several amino acid residues and having an ability to induce helper T cells.

18. The compound or pharmaceutically acceptable salt thereof of any one of items 1-17, wherein the MHC class II molecule is selected from the group consisting of DRB1*0101, DRB1*0405, DRB1*0802, DRB1*0803, DRB1*0901, DRB1*1201, DRB1*1403, DRB1*1501, DSB1*1502, DPB1*0201, DPB1*0202, DPB1*0402, DPB1*0501, DPB1*0901, DQB1*0301, DQB1*0302, DQB1*0401, DQB1*0501, DQB1*0601, DQB1*0602, and DRB5*0102.

19. The compound or pharmaceutically acceptable salt thereof of item 18, wherein the MHC class II molecule is selected from the group consisting of DRB1*0101, DRB1*0405, DSB1*1502, DPB1*0201, DPB1*0202, and DQB1*0601.

20. The compound or pharmaceutically acceptable salt thereof of any one of items 1-9, wherein R¹ is the group of formula (3).

21. The compound or pharmaceutically acceptable salt thereof of any one of items 1-9 and 20, wherein X^{b} is a divalent peptide group consisting of two amino acid residues, and Y^{b} is a single bond; X^{b} and Y^{b} are independently a divalent peptide group consisting of one amino acid residue; X^{b} is a single bond, and Y^{b} is a divalent peptide group consisting of two amino acid residues; X^{b} is a divalent peptide group consisting of one amino acid residue, and Y^{b} is a single bond; X^{b} is a single bond, and Y^{b} is a divalent peptide group consisting of one amino acid residue; or X^{b} and Y^{b} are single bonds.

22. The compound or pharmaceutically acceptable salt thereof of item 21, wherein X^{b} is a single bond and Y^{b} is a single bond or a divalent peptide group consisting of one amino acid residue.

23. The compound or pharmaceutically acceptable salt thereof of item 21, wherein X^{b} is a single bond or a divalent peptide group consisting of one amino acid residue and Y^{b} is a single bond.

24. The compound or pharmaceutically acceptable salt thereof of item 21, wherein X^{b} and Y^{b} are single bonds.

25. The compound or pharmaceutically acceptable salt thereof of any one of items 1-9 and 20-24, wherein the cancer antigen peptide C is an MHC class II-restricted WT1 peptide consisting of 9 to 30 amino acid residues.

26. The compound or pharmaceutically acceptable salt thereof of any one of items 1-9 and 20-25, wherein the cancer antigen peptide C is an MHC class II-restricted WT1 peptide consisting of 10 to 25 amino acid residues.

27. The compound or pharmaceutically acceptable salt thereof of any one of items 1-9 and 20-26, wherein the cancer antigen peptide C is a peptide comprising an amino acid sequence selected from:

(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
and
(SEQ ID NO: 248)
RYFKLSHLQMHSRKHTGE

or a peptide comprising an amino acid sequence that differs from the amino acid sequence selected from SEQ ID NOS: 217-248 by alteration of one or several amino acid residues and having an ability to induce helper T cells.

28. The compound or pharmaceutically acceptable salt thereof of any one of items 1-9 and 20-27, wherein the MHC class II molecule is selected from the group consisting of DRB1*0101, DRB1*0405, DRB1*0802, DRB1*0803, DRB1*0901, DRB1*1201, DRB1*1403, DRB1*1501, DSB1*1502, DPB1° 0201, DPB1*0202, DPB1*0402, DPB1*0501, DPB1*0901, DQB1*0301, DQB1*0302, DQB1*0401, DQB1*0501, DQB1*0601, DQB1*0602, and DRB5*0102.

29. The compound or pharmaceutically acceptable salt thereof of item 28, wherein the MHC class II molecule is selected from the group consisting of DRB1*0101, DRB1*0405, DSB1*1502, DPB1*0201, DPB1*0202, and DQB1*0601.

30. The compound or pharmaceutically acceptable salt thereof of any one of items 1-9, wherein R¹ is the cancer antigen peptide D.

31. The compound or pharmaceutically acceptable salt thereof of any one of items 1-9 and 30, wherein the cancer antigen peptide D is an MHC class II-restricted WT1 peptide consisting of 9 to 30 amino acid residues.

32. The compound or pharmaceutically acceptable salt thereof of item 31, wherein the cancer antigen peptide D is an MHC class II-restricted WT1 peptide consisting of 10 to 25 amino acid residues.

33. The compound or pharmaceutically acceptable salt thereof of item 32, wherein the cancer antigen peptide D is a peptide comprising an amino acid sequence containing at least one cysteine residue selected from:

(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
and
(SEQ ID NO: 248)
RYFKLSHLQMHSRKHTGE,

or a peptide comprising an amino acid sequence that differs from the amino acid sequence selected from SEQ ID NOS: 217-248 by alteration of one or several amino acid residues and contains at least one cysteine residue and having an ability to induce helper T cells.

34. The compound or pharmaceutically acceptable salt thereof of any one of items 1-10 and 30-33, wherein the MHC class II molecule is selected from the group consisting of DRB1*0101, DRB1*0405, DRB1*0802, DRB1*0803, DRB1*0901, DRB1*1201, DRB1*1403, DRB1*1501, DSB1*1502, DPB1*0201, DPB1*0202, DPB1*0402, DPB1*0501, DPB1*0901, DQB1*0301, DQB1*0302, DQB1*0401, DQB1*0501, DQB1*0601, DQB1*0602, and DRB5*0102.

35. The compound or pharmaceutically acceptable salt thereof of item 34, wherein the MHC class II molecule is selected from the group consisting of DRB1*0101, DRB1′0405, DSB1*1502, DPB1*0201, DPB1*0202, and DQB1*0601.

36. The compound or pharmaceutically acceptable salt thereof of item 1, wherein the compound of formula (1) is a compound of formula (4):

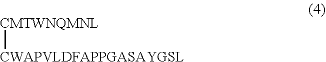

wherein C—C shown in the formula means that the C residues are linked together by a disulfide bond.

37. The compound or pharmaceutically acceptable salt thereof of item 1, wherein the compound of formula (1) is a compound of formula (5):

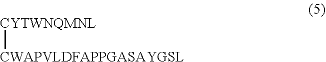

wherein C—C shown in the formula means that the C residues are linked together by a disulfide bond.

38. The compound or pharmaceutically acceptable salt thereof of item 1, wherein the compound of formula (1) is a compound of formula (6):

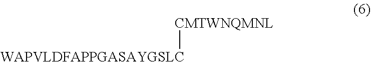

wherein C—C shown in the formula means that the C residues are linked together by a disulfide bond.

39. The compound or pharmaceutically acceptable salt thereof of item 1, wherein the compound of formula (1) is a compound of formula (7):

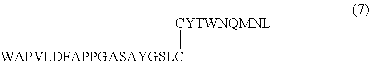

wherein C—C shown in the formula means that the C residues are linked together by a disulfide bond.

40. A composition comprising the compound or pharmaceutically acceptable salt thereof of any one of items 1-39 and at least one cancer antigen peptide E or a pharmaceutically acceptable salt thereof, wherein the at least one cancer antigen peptide E is an MHC class I-restricted WT1 peptide consisting of 7 to 30 amino acid residues.

41. The composition of item 40, wherein the at least one cancer antigen peptide E is a peptide consisting of 7 to 15 amino acid residues and restricted to at least one HLA type selected from the group consisting of HLA-A1, HLA-A2, HLA-A3, HLA-A11, HLA-A24, HLA-A28, HLA-A29, HLA-A31, HLA-A33, HLA-A34, HLA-A68.1, HLA-A1101, HLA-A0201, HLA-A0205, HLA-A3101, HLA-A3302, HLA-B7, HLA-B8, HLA-B13, HLA-B14, HLA-B35, HLA-B40, HLA-B60, HLA-B61, HLA-B62, HLA-B2702, HLA-B2705, HLA-B3501, HLA-B3701, HLA-B3801, HLA-B3901, HLA-B3902, HLA-B4403, HLA-B5101, HLA-B5102, HLA-B5201, HLA-B5801, HLA-Cw2, HLA-Cw3, HLA-Cw6, HLA-Cw7, HLA-Cw8, or HLA-Cw16, HLA-Cw0301, HLA-Cw0401, HLA-Cw0602, and HLA-Cw0702.

42. The composition of item 40 or 41, wherein the at least one cancer antigen peptide E is an MHC class I-restricted WT1 peptide consisting of 7 to 12 amino acid residues.

43. The composition of item 42, wherein the at least one cancer antigen peptide E is an MHC class I-restricted WT1 peptide consisting of 8 to 10 amino acid residues.

44. The composition of item 43, wherein the at least one cancer antigen peptide E is an MHC class I-restricted WT1 peptide consisting of 9 amino acid residues.

45. The composition of any one of items 40-44, wherein the at least one cancer antigen peptide E is a peptide different from the cancer antigen peptide A.

46. The composition of any one of items 40-45, wherein the at least one cancer antigen peptide E includes a peptide comprising an amino acid sequence selected from:

(SEQ ID NO: 2)
RMFPNAPYL,
(SEQ ID NO: 209)
YMFPNAPYL,
(SEQ ID NO: 3)
CMTWNQMNL,
(SEQ ID NO: 4)
CYTWNQMNL,
(SEQ ID NO: 5)
ALLPAVPSL,
(SEQ ID NO: 6)
SLGEQQYSV,
and
(SEQ ID NO: 7)
RVPGVAPTL

or a peptide comprising an amino acid sequence that differs from the amino acid sequence selected from SEQ ID NOS: 2, 3, and 5-7 by alteration of one or several amino acid residues and having an ability to induce CTLs.

47. A composition comprising at least one compound selected from the group consisting of a compound of formula (4):

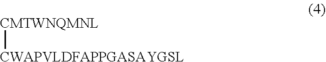

wherein C—C shown in the formula means that the C residues are linked together by a disulfide bond, a compound of formula (5):

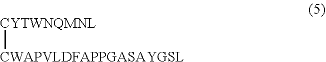

wherein C—C shown in the formula means that the C residues are linked together by a disulfide bond, a compound of formula (6):

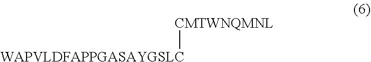

wherein C—C shown in the formula means that the C residues are linked together by a disulfide bond, and a compound of formula (7):

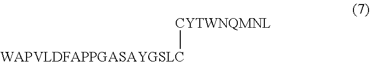

wherein C—C shown in the formula means that the C residues are linked together by a disulfide bond, or a pharmaceutically acceptable salt thereof, and at least one peptide consisting an amino acid sequence selected from:

(SEQ ID NO: 2)
RMFPNAPYL,
(SEQ ID NO: 209)
YMFPNAPYL,
(SEQ ID NO: 3)
CMTWNQMNL,
(SEQ ID NO: 4)
CYTWNQMNL,
(SEQ ID NO: 5)
ALLPAVPSL,
(SEQ ID NO: 6)
SLGEQQYSV,
and
(SEQ ID NO: 7)
RVPGVAPTL

or a pharmaceutically acceptable salt thereof.

48. A pharmaceutical composition comprising the compound or pharmaceutically acceptable salt thereof of any one of items 1-39, or the composition of any one of claims 40-47, and a pharmaceutically acceptable carrier.

49. The pharmaceutical composition of item 48, wherein the compound or pharmaceutically acceptable salt thereof of any one of items 1-39 is used in combination with at least one cancer antigen peptide E, wherein the at least one cancer antigen peptide E is an MHC class I-restricted peptide consisting of 7 to 30 amino acid residues.

50. The pharmaceutical composition of item 49, wherein the compound or pharmaceutically acceptable salt thereof and the at least one cancer antigen peptide E are administered concurrently.

51. The pharmaceutical composition of item 49, wherein the compound or pharmaceutically acceptable salt thereof is administered before the at least one cancer antigen peptide E is administered.

52. The pharmaceutical composition of item 49, wherein the compound or pharmaceutically acceptable salt thereof is administered after the at least one cancer antigen peptide E is administered.

53. The pharmaceutical composition of any one of items 48-52, wherein the pharmaceutical composition is for use as a composition for treating a cancer associated with WT1 gene expression or an elevated level of WT1 gene expression.

54. The pharmaceutical composition of any one of items 48-52, wherein the pharmaceutical composition is for use as a composition for inducing CTLs in cellular immunotherapy for a cancer.

55. The pharmaceutical composition of any one of items 48-52, wherein the pharmaceutical composition is for use as a cancer vaccine.

56. The pharmaceutical composition of any one of items 53-55, wherein the cancer is a hematologic cancer or a solid cancer.

57. The pharmaceutical composition of item 56, wherein the cancer is a hematologic cancer selected from leukemia, myelodysplastic syndrome, multiple myeloma, and malignant lymphoma.

58. The pharmaceutical composition of item 56, wherein the cancer is a solid cancer selected from gastric cancer, colorectal cancer, lung cancer, breast cancer, germ cell cancer, liver cancer, skin cancer, urinary bladder cancer, prostate cancer, uterine cancer, cervical cancer, ovarian cancer, and brain tumor.

59. Use of the compound or pharmaceutically acceptable salt thereof of any one of items 1-39, the composition of any one of items 40-47, or the pharmaceutical composition of any one of items 48-58 for the manufacture of a cancer vaccine.

60. A method of treating or preventing a cancer, comprising administering to a WT1-positive subject in need thereof a therapeutically or prophylactically effective amount of the compound or pharmaceutically acceptable salt thereof of any one of items 1-39, the composition of any one of items 40-47, or the pharmaceutical composition of any one of items 48-58.

61. A method of obtaining an MHC class I-restricted peptide and an MHC class II-restricted peptide, comprising reacting the compound or pharmaceutically acceptable salt thereof of any one of items 1-39, the composition of any one of items 40-47, or the pharmaceutical composition of any one of items 48-58 with ERAP1.

62. A method of synthesizing the compound of formula (1), comprising forming a disulfide bond between the cysteine residue of the cancer antigen peptide A as described in any one of items 1-19, 36 and 37 and a cysteine residue attached to the N-terminus of the cancer antigen peptide B as described in any one of the items.

63. A method of synthesizing the compound of formula (1), comprising forming a disulfide bond between the cysteine residue of the cancer antigen peptide A as described in any one of items 1-9, 20-29, 38 and 39 and a cysteine residue attached to the C-terminus of the cancer antigen peptide C as described in any one of the items.

64. A method for synthesizing the compound of formula (1), comprising forming a disulfide bond between the cysteine residue of the cancer antigen peptide A as described in any one of items 1-9 and 30-35 and the cysteine residue of the cancer antigen peptide D as described in any one of the items.

65. An MHC class I-restricted peptide consisting of 7 to 30 amino acid residues for use in combination with the compound or pharmaceutically acceptable salt thereof of any one of items 1-39, the composition of any one of items 40-47, or the pharmaceutical composition of any one of items 48-58.

66. The compound or pharmaceutically acceptable salt thereof of any one of items 1-39, the composition of any one of items 40-47, or the pharmaceutical composition of any one of items 48-58, for use in combination with at least one MHC class I-restricted peptide consisting of 7 to 30 amino acid residues.

67. A kit for treating a cancer, wherein the kit comprises at least one MHC class I-restricted peptide consisting of 7 to 30 amino acid residues and the compound or pharmaceutically acceptable salt thereof of any one of items 1-39, the composition of any one of items 40-47, or the pharmaceutical composition of any one of items 48-58.

### Effects of Invention

The present disclosure provides the compound of formula (1) and a composition comprising the compound, which are useful for treating or preventing a cancer.

### BRIEF DESCRIPTION OF DRAWINGS

FIG. 1 shows results of testing a CTL inducing ability in vivo in an HLA-A2402 transgenic mouse with the peptide of SEQ ID NO: 4 synthesized in Reference Example 2 and the compound of formula (5) synthesized in Example 1 in an IFNγ ELISPOT assay of Experimental Example 1.

FIG. 2 shows results of testing an ability to induce IFNγ-producing cells in vivo in an HLA-A0201 transgenic mouse with the peptide of SEQ ID NO: 2 synthesized in Reference Example 1 and the compound of formula (5) synthesized in Example 1 in an IFNγ ELISPOT assay of Experimental Example 2.

### DESCRIPTION OF EMBODIMENTS

Embodiments of the present application are described in detail below.

An “amino acid residue” as used herein refers to a single amino acid unit among amino acids constituting a peptide or protein molecule. An “amino acid residue” may be a natural or non-natural α-amino acid residue, β-amino acid residue, γ-amino acid residue or δ-amino acid residue, more specifically, a natural α-amino acid residue, ornithine residue, homoserine residue, homocysteine residue, β-alanine residue, γ-aminobutanoic acid or δ-aminopentanoic acid. When an “amino acid residue” is optically active, it includes L-form and D-form, and is preferably L-form.

For describing an “amino acid residue”, an abbreviation for it may be used. The following is a list of the abbreviations:

Ala or A: alanine residue
a: D-alanine residue
Arg or R: arginine residue
Asn or N: asparagine residue
Asp or D: aspartic acid residue
Cys or C: cysteine residue
Gln or Q: glutamine residue
Glu or E: glutamic acid residue
Gly or G: glycine residue
His or H: histidine residue
Ile or I: isoleucine residue
Leu or L: leucine residue
Lys or K: lysine residue
Met or M: methionine residue
Phe or F: phenylalanine residue
Pro or P: proline residue
Ser or S: serine residue
Thr or T: threonine residue
Trp or W: tryptophan residue
Tyr or Y: tyrosine residue
Val or V: valine residue
Abu: 2-aminobutyric acid residue (also referred to as α-aminobutyric acid residue)
Orn: ornithine residue
Cit: citrulline residue
Cha: Cyclohexylalanine residue
Ahx: 2-Aminohexanoic acid residue

An amino acid sequence of a “peptide” is described herein so that its N-terminal amino acid residue is positioned on the left side, and its C-terminal amino acid residue is positioned on the right side in accordance with a usual description method. Unless otherwise indicated, in a “peptide”, the amino group of the N-terminal amino acid binds to a hydrogen atom (to be a free amino group) and the carbonyl group of the C-terminal amino acid binds to a hydroxyl group. A divalent peptide group means a peptide group that is able to bind to other chemical moieties via the N-terminal amino group and via the C-terminal carbonyl group. Unless otherwise indicated, in a peptide corresponding to partial structure of a compound of formula (1), for example, the compound of any one of the formulae (4)-(7), the amino group of the N-terminal amino acid binds to a hydrogen atom and the carbonyl group of the C-terminal amino acid binds to a hydroxyl group.

“Cancer antigen peptide A” is an MHC class I-restricted peptide consisting of 7 to 30 amino acid residues and containing at least one cysteine residue. In formula (1), the cysteine residue of the cancer antigen peptide A binds to R¹ via a disulfide bond.

In formula (1), “R¹” is hydrogen, a group of formula (2), a group of formula (3), or cancer antigen peptide D. Preferably, R¹ is a group of formula (2), a group of formula (3), or cancer antigen peptide D, more preferably a group of formula (2) or a group of formula (3).

When R¹ is a group of formula (2), the compound of formula (1) is a compound of formula (1-1):

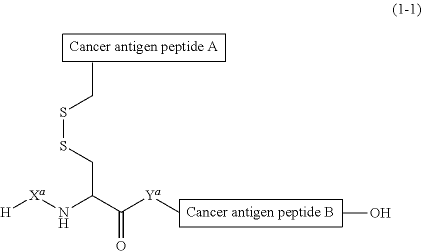

wherein X^{a}, Y^{a} and cancer antigen peptide B have the same meanings as defined above in relation to formula (2).

“X^{a}” and “Y^{a}” independently represent a single bond or a divalent peptide group consisting of 1 to 4 amino acid residues, provided that the sum of the number of amino acid residues in X^{a} and Y^{a} is an integer of 0 to 4. For example, when the sum of the number of amino acid residues in X^{a} and Y^{a} is an integer of 0, both X^{a} and Y^{a} must be single bonds; and when the sum of the number of amino acid residues in X^{a} and Y^{a} is an integer of 4, each of X^{a} and Y^{a} may be a divalent peptide group consisting of two amino acid residues, or X^{a} may be a divalent peptide group consisting of three amino acid residues, and Y^{a} may be a divalent peptide group consisting of one amino acid residue, or X^{a} may be a divalent peptide group consisting of four amino acid residues, and Y^{a} may be a single bond.

The sum of the number of amino acid residues in X^{a} and Y^{a} is preferably an integer of 0 to 2, more preferably an integer of 0 to 1, or most preferably zero. That is, most preferably, X^{a} and Y^{a} are both single bonds.

When the sum of the number of amino acid residues in X^{a} and Y^{a} is an integer of 2, X^{a} may be a divalent peptide group consisting of two amino acid residues, and Y^{a} may be a single bond; X^{a} and Y^{a} may independently be a divalent peptide group consisting of one amino acid residue; or X^{a} may be a single bond, and Y^{a} is a divalent peptide group consisting of two amino acid residues.

When the sum of the number of amino acid residues in X^{a} and Y^{a} is an integer of 1, X^{a} may be a divalent peptide group consisting of one amino acid residue, and Y^{a} may be a single bond; or X^{a} may be a single bond, and Y^{a} may be a divalent peptide group consisting of one amino acid residue. In a preferred embodiment, X^{a} is a single bond, and Y^{a} is a residue of alanine, leucine or methionine; or X^{a} is a residue of alanine, leucine or methionine, and Y^{a} is a single bond.

“Cancer antigen peptide B” is an MHC class II-restricted peptide consisting of 9 to 30 amino acid residues. In formula (2) (or formula (1-1)), the amino group of the N-terminal amino acid of the cancer antigen peptide B binds to Y^{a} in the formula (2), and the carbonyl group of the C-terminal amino acid of the cancer antigen peptide B binds to the hydroxyl group in the formula (2).

When R¹ is a group of formula (3), the compound of formula (1) is a compound of formula (1-2):

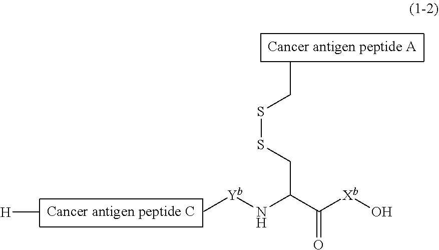

wherein X^{b}, Y^{b} and cancer antigen peptide C have the same meanings as defined above in relation to formula (3).

“X^{b}” and “Y^{b}” independently represent a single bond or a divalent peptide group consisting of 1 to 4 amino acid residues, provided that the sum of the number of amino acid residues in X^{b} and Y^{b} is an integer of 0 to 4. For example, when the sum of the number of amino acid residues in X and Y^{b} is an integer of 0, both X^{b} and Y^{b} must be single bonds; and when the sum of the number of amino acid residues in X^{b} and Y^{b} is an integer of 4, each of X^{b} and Y^{b} may be a divalent peptide group consisting of two amino acid residues, or X^{b} may be a divalent peptide group consisting of three amino acid residues, and Y^{b} may be a divalent peptide group consisting of one amino acid residue, or X^{b} may be a divalent peptide group consisting of four amino acid residues, and Y^{b} may be a single bond.

The sum of the number of amino acid residues in X^{b} and Y^{b} is preferably an integer of 0 to 2, more preferably an integer of 0 to 1, or most preferably zero. That is, most preferably, X^{b} and Y^{b} are both single bonds.

For example, when the sum of the number of amino acid residues in X^{b} and Y^{b} is an integer of 2, X^{b} may be a divalent peptide group consisting of two amino acid residues, and Y^{b} may be a single bond, each of X^{b} and Y^{b} may independently be a divalent peptide group consisting of one amino acid residue, or X^{b} may be a single bond, and Y^{b} may be a divalent peptide group consisting of two amino acid residues.

When the sum of the number of amino acid residues in X^{b} and Y^{b} is an integer of 1, X^{b} may be a divalent peptide group consisting of one amino acid residue, and Y^{b} may be a single bond, or X^{b} may be a single bond, and Y^{b} may be a divalent peptide group consisting of one amino acid residue. In a preferred embodiment, X^{b} is a single bond, and Y^{b} is a residue of alanine, leucine or methionine, or X^{b} is a residue of alanine, leucine or methionine, and Y^{b} is a single bond.

“Cancer antigen peptide C” is an MHC class II-restricted peptide consisting of 9 to 30 amino acid residues. In formula (3) (or formula (1-2)), the carbonyl group of the C-terminal amino acid of the cancer antigen peptide C binds to Y^{b} in the formula (3), and the amino group of the N-terminal amino acid of the cancer antigen peptide C binds to the hydrogen atom in the formula (3).

When R¹ is cancer antigen peptide D, the compound of formula (1) is a compound of formula (1-3):

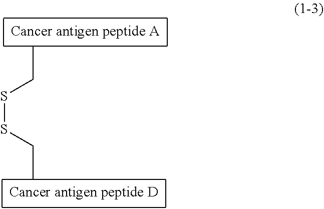

wherein cancer antigen peptide D have the same meanings as defined above.

“Cancer antigen peptide D” is an MHC class II-restricted peptide consisting of 9 to 30 amino acid residues and containing at least one cysteine. When R¹ is the cancer antigen peptide D, the cysteine residue of the cancer antigen peptide D binds to R¹ via a disulfide bond.

Cancer antigen peptide D contains at least one cysteine residue in its amino acid sequence. The number of cysteine residue(s) is preferably 1 to 3, more preferably 1 to 2, or most preferably one.

As used herein, the term “WT1 peptide”, which is synonymous with “partial peptide of WT1 protein”, refers to a peptide consisting of contiguous amino acid residues of the amino acid sequence of human WT1 protein of SEQ ID NO: 1, or an altered peptide thereof having an ability to induce CTLs or helper T cells.

The term “MHC class I-restricted” means an ability of a peptide to bind to a Major Histocompatibility Complex (MHC) class I molecule and induce CTLs.

MHC of human is called human leukocyte-type antigen (HLA). HLA molecules corresponding to MHC class I-molecules include HLA-A, B, Cw, F and G subtypes. Restriction to HLA-A, HLA-B, or HLA-Cw is preferred as the restriction to MHC class I of an “MHC class I-restricted” peptide.

Allelic polymorphism is known for each HLA subtype. For HLA-A, 27 or more types of polymorphism including HLA-A1, HLA-A0201, and HLA-A24 are known. For HLA-B, 59 or more types of polymorphism including HLA-B7, HLA-B40, and HLA-B4403 are known. For HLA-Cw, 10 or more types of polymorphism including HLA-Cw0301, HLA-Cw0401, and HLA-Cw0602 are known. Among such polymorphism, HLA-A0201 and HLA-A24 are preferred.

The term “MHC class I-restricted peptide” as used herein refers to a peptide being capable of binding to an MHC class I molecule to be presented in a form of a complex and inducing CTLs from precursor T cells that recognize the complex in vitro and/or in vivo (in other words, a peptide having an ability to induce CTLs.). An “MHC class I-restricted WT1 peptide” is a WT1 peptide being an “MHC class I-restricted peptide”. The “MHC class I-restricted peptide” and “MHC class I-restricted WT1 peptide” are often herein referred to as “killer peptide” and “WT1 killer peptide”, respectively. The “MHC class I-restricted peptide” may consists of a sequence of any number of amino acids of any type, so long as it functions as the “MHC class I-restricted peptide” as defined above. However, the longer a peptide chain is, the more susceptible it may be to degradation by a proteolytic enzyme. Also, too small peptide may not successfully be caught in a peptide-binding groove of an MHC class I molecule. The “MHC class I-restricted peptide” typically consists of 7 to 30 amino acid residues, preferably 7 to 15 amino acid residues, more preferably 8 to 12 amino acid residues, still more preferably 8 to 11 amino acid residues, or most preferably 8 or 9 amino acid residues.

An “MHC class I-restricted peptide” consisting of 7 to 12 amino acid residue, preferably 9 amino acid residue, may also be referred to as an “MHC class I-restricted epitope”. The term “MHC class I-restricted epitope” refers to a peptide corresponding to an actual peptide complexed with an MHC class I molecule and presented. That is, the term “MHC class I-restricted peptide” includes a peptide that provides an “MHC class I-restricted epitope” through a process such as degradation with proteasome and/or protease, and/or cutting (also referred to as trimming) by ERAP1 to an appropriate peptide length in vitro or in vivo.

An “MHC class I-restricted epitope” may be derived from an “MHC class I-restricted peptide” by degradation with proteasome and/or protease, followed by trimming (cutting) by ERAP1, wherein a C-terminal amino acid residue and an N-terminal amino acid residue of the “MHC class I-restricted epitope” may be determined by the action of proteasome/protease and ERAP1, respectively. Accordingly, the “MHC class I-restricted peptide” may be a peptide consisting of 7 to 30 amino acid residues wherein 0 to 23 amino acid residues are attached to a “MHC class I-restricted epitope” consisting of 7 to 12 residues, via its C-terminal carbonyl group.

As used herein, the term “peptide comprising an amino acid sequence” refers to a peptide having a given amino acid sequence, which may, as usually understood, optionally have extra sequence(s) of amino acid residue(s) attached to the N-terminal and/or C-terminal amino acid of the given sequence.

As used herein, the term “altered peptide” refers to a peptide consisting of an amino acid sequence that differs from the amino acid sequence of the original peptide by alteration of one or several amino acid residues. In an altered peptide, one or several amino acid residues, for example, 1 to 9 amino acid residues, preferably 1 to 5, 1 to 4 or 1 to 3 amino acid residues, more preferably 1 to 2 amino acid residues, or most preferably one amino acid residue is deleted from, substituted in, and/or added (or inserted) to the amino acid sequence of the original peptide. The number of amino acid(s) deleted from, substituted in, and/or added (or inserted) to the amino acid sequence of the original peptide may preferably be 1 to 5, 1 to 4, or 1 to 3, more preferably 1 to 2, or most preferably one. Amino acid substitution for altering a peptide may be made at any position of amino acid residue in the original sequence with any type of amino acid. Conservative amino acid substitution is preferred. For example, substitution of Asp for Glu; Tyr for Phe; Ile for Leu; Ser for Ala; or Arg for His may be made. Amino acid addition or deletion may preferably be made at N- or C-terminus of a peptide. However, amino acid addition or deletion may be made internally. Amino acid addition (or insertion) or substitution may be made with any of the twenty genetically encoded amino acids or even any non-natural amino acid.

As used herein, a killer peptide consisting of an altered amino acid sequence is also referred to as an “altered killer peptide”. In an altered killer peptide, amino acid substitution may be made, specifically at amino acid position 1 (N-terminus), 2, 3 or C-terminus, for example at position 1 (N-terminus), 2, 3 or 9 (C-terminus) in a peptide consisting of nine amino acid residues. When an altered killer peptide has added (or inserted) amino acid residue(s), the number of added amino acid(s) is preferably 1 or 2, or more preferably one. Amino acid addition is preferably made to N-terminus or C-terminus, more preferably to C-terminus. When a killer peptide is altered by amino acid deletion, the number of deleted amino acid(s) is preferably 1 or 2, or more preferably one. Amino acid deletion is preferably made at N-terminus or C-terminus, more preferably at C-terminus.

Each HLA subtype carries polymorphism. Peptides that can complex with a polymorphic sequence of an HLA antigen have a specific pattern of amino acid sequence (that is, binding motif) for binding to the polymorphic sequence of the HLA antigen. Amino acid substitution may be made at any of amino acid residues constituting such a binding motif. For example, an HLA-A24-binding peptide that consists of 8 to 11 amino acid residues is known to have Tyr, Phe, Met or Trp at position 2, and Phe, Leu, Ile, Trp or Met at the C-terminus (J. Immunol., 152, p 3913, 1994; J. Immunol., 155, p 4307, 1994; Immunogenetics, 41, p 178, 1995). Therefore, for example, a peptide consisting of nine amino acid residues may be altered by amino acid substitution to have Tyr, Phe, Met or Trp at position 2, and/or Phe, Leu, Ile, Trp or Met at position 9 (C-terminus) to give an altered peptide useful as an altered killer peptide. Also, an HLA-A0201-binding peptide that consists of 8 to 11 amino acid residues is known to have Leu or Met at position 2, and Val or Leu at the C-terminus. Therefore, for example, a peptide consisting of nine amino acid residues may be altered by amino acid substitution to have Leu or Met at position 2, and/or Val or Leu at position 9 (C-terminus) to give an altered peptide useful as an altered killer peptide.

Examples of the “MHC class I-restricted WT1 peptide” include the peptides as shown in Tables 1-44. In the tables, the column “position” shows positions in the amino acid sequence of human WT1 of SEQ ID NO: 1 to which each peptide corresponds.

TABLE 1

| | Amino acid | SEQ | |
|---|---|---|---|
| Position | sequence | ID NO: | HLA subtype |
| 2-10 | GSDVRDLNA | 8 | A1 |
| 3-11 | SDVRDLNAL | 9 | B40, B60, B61, |
| | | | B3701, B4403, |
| | | | Cw0301, Cw0602 |
| 4-12 | DVRDLNALL | 10 | A24, A68.1, |
| | | | A3302, B7, B8, |
| | | | B3501, B3701, |
| | | | Cw0401, Cw0602 |
| 6-14 | RDLNALLPA | 11 | B40, B61, B3701 |
| 7-15 | DLNALLPAV | 12 | A0201, B62, B5201 |
| 10-18 | ALLPAVPSL | 5 | A0201, A0205, A24, |
| | | | A3, B14, B7, B8, |
| | | | B3801, B3901, |
| | | | B3902, Cw0301, |
| | | | Cw0401, Cw0602 |

TABLE 2

| | Amino acid | SEQ | |
|---|---|---|---|
| Position | sequence | ID NO: | HLA subtype |
| 17-25 | SLGGGGGCA | 13 | B62 |
| 18-26 | LGGGGGCAL | 14 | B60, B7, B3801, |
| | | | B5101, B5102 |
| 20-28 | GGGGCALPV | 15 | B61, B5101, |
| | | | B5102, B5201 |

TABLE 3

| | Amino acid | SEQ | |
|---|---|---|---|
| Position | sequence | ID NO: | HLA subtype |
| 23-31 | GCALPVSGA | 16 | B40, B61 |
| 24-32 | CALPVSGAA | 17 | B40, B5102, |
| | | | Cw0301 |
| 26-34 | LPVSGAAQW | 18 | B40, B3501, |
| | | | B5801 |
| 29-37 | SGAAQWAPV | 19 | B5101, B5102, |
| | | | B5201, B61 |
| 30-38 | GAAQWAPVL | 20 | B40, B60, B7, |
| | | | B8, B3902, |
| | | | B5101, B5102, |
| | | | Cw0301, Cw0602 |

TABLE 4

| | Amino acid | SEQ | |
|---|---|---|---|
| Position | sequence | ID NO: | HLA subtype |
| 32-40 | AQWAPVLDF | 21 | A3, A3101, |
| | | | B62, B2702, |
| | | | B2705, B3902, |
| | | | B5201 |
| 33-41 | QWAPVLDFA | 22 | Cw0702 |
| 37-45 | VLDFAPPGA | 23 | A1, A0201 |
| 38-46 | LDFAPPGAS | 24 | B40, B3701 |
| 39-47 | DFAPPGASA | 25 | Cw0401 |
| 40-48 | FAPPGASAY | 26 | A1, B62, |
| | | | B3501, B4403, |
| | | | B5801, Cw0702 |

TABLE 5

| | Amino acid | SEQ | |
|---|---|---|---|
| Position | sequence | ID NO: | HLA subtype |
| 47-55 | AYGSLGGPA | 27 | A24 |

TABLE 6

| | Amino acid | SEQ | |
|---|---|---|---|
| Position | sequence | ID NO: | HLA subtype |
| 63-71 | PPPPPPHSF | 28 | Cw0401 |
| 64-72 | PPPPPHSFI | 29 | B5101, B5102, |
| | | | B5201 |
| 65-73 | PPPPHSFIK | 30 | A1101 |
| 70-78 | SFIKQEPSW | 31 | Cw0401 |

TABLE 7

| | Amino acid | SEQ | |
|---|---|---|---|
| Position | sequence | ID NO: | HLA subtype |
| 73-81 | KQEPSWGGA | 32 | A1, A0205 |
| 80-88 | GAEPHEEQC | 33 | A1 |

TABLE 8

| | Amino acid | SEQ | |
|---|---|---|---|
| Position | sequence | ID NO: | HLA subtype |
| 81-89 | AEPHEEQCL | 34 | A0205, B40, |
| | | | B60, B61, |
| | | | B3701, B4403 |
| 82-90 | EPHEEQCLS | 35 | B3501, B5101 |
| 83-91 | PHEEQCLSA | 36 | B3801 |
| 84-92 | HEEQCLSAF | 37 | B40, B3701, |
| | | | B4403, |
| | | | Cw0702 |
| 85-93 | EEQCLSAFT | 38 | B40, B60, |
| | | | B61, B3701, |
| | | | B4403 |
| 86-94 | EQCLSAFTV | 39 | A0201, B62, |
| | | | B5201 |
| 88-96 | CLSAFTVHF | 40 | A3, B62 |

TABLE 9

| | Amino acid | SEQ | |
|---|---|---|---|
| Position | sequence | ID NO: | HLA subtype |
| 92-100 | FTVHFSGQF | 41 | B62, B5801, |
| | | | Cw0301 |
| 93-101 | TVHFSGQFT | 42 | A0201, A0205 |
| 96-104 | FSGQFTGTA | 43 | B5801, B4403 |
| 98-106 | GQFTGTAGA | 44 | A0205, B40, |
| | | | B62, B2702, |
| | | | B5201 |
| 99-107 | QFTGTAGAC | 45 | Cw0401 |
| 100-108 | FTGTAGACR | 46 | A68.1, A1101, |
| | | | A3101, A3302 |

TABLE 10

| | Amino acid | SEQ | |
|---|---|---|---|
| Position | sequence | ID NO: | HLA subtype |
| 101-109 | TGTAGACRY | 47 | B62, B4403, |
| | | | Cw0702 |
| 104-112 | AGACRYGPF | 48 | B4403, B5201 |
| 107-115 | CRYGPFGPP | 49 | B2702 |
| 110-118 | GPFGPPPPS | 50 | B5101, B5102 |

TABLE 11

| | Amino acid | SEQ | |
|---|---|---|---|
| Position | sequence | ID NO: | HLA subtype |
| 118-126 | SQASSGQAR | 51 | A68.1, A1101, |
| | | | A3101, A3302 |
| 119-127 | QASSGQARM | 52 | B3501, B5101, |
| | | | B5102 |
| 120-128 | ASSGQARMF | 53 | B3501, B3801, |
| | | | B4403, B5801 |

TABLE 12

| | Amino acid | SEQ | |
|---|---|---|---|
| Position | sequence | ID NO: | HLA subtype |
| 123-131 | GQARMFPNA | 54 | B62 |
| 125-133 | ARMFPNAPY | 55 | B14, B2702, |
| | | | B2705, Cw0702 |
| 126-134 | RMFPNAPYL | 2 | A0201, A0205, |
| | | | A24, A3, B14, |
| | | | B7, B2702, |
| | | | B2705, B3901, |
| | | | B3902, Cw0301 |
| 128-136 | FPNAPYLPS | 56 | B5101 |
| 130-138 | NAPYLPSCL | 57 | A24, B60, B7, |
| | | | B8, B3902, B5101, |
| | | | B5102, Cw0301, |
| | | | Cw0602, Cw0702 |

TABLE 13

| | Amino acid | SEQ | |
|---|---|---|---|
| Position | sequence | ID NO: | HLA subtype |
| 136-144 | SCLESQPAI | 58 | B8, B3901, |
| | | | B5102, Cw0301 |
| 137-145 | CLESQPAIR | 59 | A1, A3, A68.1, |
| | | | A1101, A3101, |
| | | | A3302 |
| 138-146 | LESQPAIRN | 60 | B60, B61 |
| 139-147 | ESQPAIRNQ | 61 | A3302 |

TABLE 14

| | Amino acid | SEQ | |
|---|---|---|---|
| Position | sequence | ID NO: | HLA subtype |
| 141-149 | QPAIRNQGY | 62 | B8, B3501, |
| | | | B4403, Cw0401, |
| | | | Cw0702 |
| 143-151 | AIRNQGYST | 63 | B7 |
| 144-152 | IRNQGYSTV | 64 | B14, B2702, |
| | | | B2705, B3901 |
| 146-154 | NQGYSTVTF | 65 | B62, B2702, |
| | | | B3902, B5201 |

TABLE 15

| | Amino acid | SEQ ID | |
|---|---|---|---|
| Position | sequence | NO: | HLA subtype |
| 152-160 | VTFDGTPSY | 66 | A1, A3, B62, B3501, |
| | | | B4403, B5801, Cw0702 |

TABLE 16

| | Amino acid | SEQ ID | |
|---|---|---|---|
| Position | sequence | NO: | HLA subtype |
| 161-169 | GHTPSHHAA | 67 | B3801 |
| 163-171 | TPSHHAAQF | 68 | B3501, B3801, |
| | | | Cw0401, Cw0702 |
| 165-173 | SHHAAQFPN | 69 | B3801 |
| 168-176 | AAQFPNHSF | 70 | B5801 |
| 169-177 | AQFPNHSFK | 71 | A3, A68.1, A1101, |
| | | | A3101, B2705 |

TABLE 17

| | Amino acid | SEQ ID | |
|---|---|---|---|
| Position | sequence | NO: | HLA subtype |
| 174-182 | HSFKHEDPM | 72 | B14, B3501, B5801 |
| 177-185 | KHEDPMGQQ | 73 | B3801 |
| 179-187 | EDPMGQQGS | 74 | B3701 |
| 180-188 | DPMGQQGSL | 75 | A24, B14, B60, B7, |
| | | | B8, B3501, B3801, |
| | | | B3901, B5101, B5102, |
| | | | Cw0301, Cw0401, |
| | | | Cw0602 |

TABLE 18

| | Amino acid | SEQ ID | |
|---|---|---|---|
| Position | sequence | NO: | HLA subtype |
| 185-193 | QGSLGEQQY | 76 | B4403, Cw0702 |
| 187-195 | SLGEQQYSV | 6 | A0201, A0205, A3, |
| | | | B62 |

TABLE 19

| | Amino acid | SEQ ID | |
|---|---|---|---|
| Position | sequence | NO: | HLA subtype |
| 191-199 | QQYSVPPPV | 77 | A0201, A0205,861, |
| | | | B62, B2702, B2705, |
| | | | B5201 |
| 192-200 | QYSVPPPVY | 78 | A24, Cw0401, Cw0702 |
| 194-202 | SVPPPVYGC | 79 | A0205, A3 |

TABLE 20

| | Amino acid | SEQ ID | |
|---|---|---|---|
| Position | sequence | NO: | HLA subtype |
| 202-210 | CHTPTDSCT | 80 | B3801 |
| 204-212 | TPTDSCTGS | 81 | B5101 |
| 206-214 | TDSCTGSQA | 82 | B40, B61, B3701 |
| 207-215 | DSCTGSQAL | 83 | A24, A3302, B60, B7, |
| | | | B8, B3501, B3901, |
| | | | B3902, Cw0602 |
| 208-216 | SCTGSQALL | 84 | B60, B7, B8, B3701, |
| | | | B3801, B3901, B3902 |
| 209-217 | CTGSQALLL | 85 | B60, B7, B3701, |
| | | | B3902 |
| 210-218 | TGSQALLLR | 86 | A3302 |

TABLE 21

| | Amino acid | SEQ ID | |
|---|---|---|---|
| Position | sequence | NO: | HLA subtype |
| 211-219 | GSQALLLRT | 87 | B5801 |
| 213-221 | QALLLRTPY | 88 | A1, B3501, B4403, |
| | | | B5801, Cw0602, |
| | | | Cw0702 |
| 217-225 | LRTPYSSDN | 89 | B2702 |
| 218-226 | RTPYSSDNL | 90 | A24, B60, B7, B3902, |
| | | | B5801 |
| 219-227 | TPYSSDNLY | 91 | B3501, B5101, B5102, |
| | | | Cw0401, Cw0702 |

TABLE 22

| | Amino acid | SEQ ID | |
|---|---|---|---|
| Position | sequence | NO: | HLA subtype |
| 221-229 | YSSDNLYQM | 92 | B60, B3501 |
| 222-230 | SSDNLYQMT | 93 | A1, B5801 |
| 223-231 | SDNLYQMTS | 94 | B3701 |
| 225-233 | NLYQMTSQL | 95 | A0201, A0205, A24, |
| | | | B14, B7, B8, B3801, |
| | | | B3901, B3902, |
| | | | Cw0301, Cw0602 |
| 227-235 | YQMTSQLEC | 96 | A0201, A0205, B62 |
| 228-236 | QMTSQLECM | 97 | A0201 |
| 230-238 | TSQLECMTW | 98 | B5801 |

TABLE 23

| | Amino acid | SEQ ID | |
|---|---|---|---|
| Position | sequence | NO: | HLA subtype |
| 232-240 | QLECMTWNQ | 99 | A1 |
| 233-241 | LECMTWNQM | 100 | B40, B60, B61, |
| | | | B3701, B4403 |
| 235-243 | CMTWNQMNL | 3 | A0201, A0205, A24, |
| | | | A3, B7 |
| 239-247 | NQMNLGATL | 101 | A0201, A0205, A24, |
| | | | B14, B60, B62, B7, |
| | | | B2702, B2705, B3901, |
| | | | B3902, B5201, |
| | | | Cw0301, Cw0602 |
| 240-248 | QMNLGATLK | 102 | A24, A3, A1101, |
| | | | A3101 |

TABLE 24

| | Amino acid | SEQ ID | |
|---|---|---|---|
| Position | sequence | NO: | HLA subtype |
| 242-250 | NLGATLKGV | 103 | A0201, A0205, B62, |
| | | | Cw0602 |
| 243-251 | LGATLKGVA | 104 | B5201 |
| 244-252 | GATLKGVAA | 105 | B61, B8 |
| 250-258 | VAAGSSSSV | 106 | B61, B5101, B5102 |

TABLE 25

| | Amino acid | SEQ ID | |
|---|---|---|---|
| Position | sequence | NO: | HLA subtype |
| 251-259 | AAGSSSSVK | 107 | A68.1, A1101 |
| 252-260 | AGSSSSVKW | 108 | B5801 |
| 260-268 | WTEGQSNHS | 109 | A1 |

TABLE 26

| | Amino acid | SEQ ID | |
|---|---|---|---|
| Position | sequence | NO: | HLA subtype |
| 261-269 | TEGQSNHST | 110 | B40, B60, B61, |
| | | | B4403 |
| 263-271 | GQSNHSTGY | 111 | A3, B62, B2702, |
| | | | Cw0702 |
| 269-277 | TGYESDNHT | 112 | B5102, B5201 |
| 270-278 | GYESDNHTT | 113 | A24 |

TABLE 27

| | Amino acid | SEQ ID | |
|---|---|---|---|
| Position | sequence | NO: | HLA subtype |
| 272-280 | ESDNHTTPI | 114 | A1, A3302, B5101 |
| 273-281 | SDNHTTPIL | 115 | B40, B60, B3701, |
| | | | B5201 |
| 276-284 | HTTPILCGA | 116 | B5801 |
| 278-286 | TPILCGAQY | 117 | B3501, B4403, |
| | | | Cw0401, Cw0702 |
| 279-287 | PILCGAQYR | 118 | A3101 |
| 280-288 | ILCGAQYRI | 119 | A0201, A0205, A3, |
| | | | B62, B5101 |

TABLE 28

| | Amino acid | SEQ ID | |
|---|---|---|---|
| Position | sequence | NO: | HLA subtype |
| 285-293 | QYRIHTHGV | 120 | A24, Cw0401 |
| 286-294 | YRIHTHGVF | 121 | B14, B2702, B2705, |
| | | | B5201, Cw0301 |
| 287-295 | RIHTHGVFR | 122 | A3, A1101, A3101, |
| | | | A3302 |
| 289-297 | HTHGVFRGI | 123 | B5801 |

TABLE 29

| | Amino acid | SEQ ID | |
|---|---|---|---|
| Position | sequence | NO: | HLA subtype |
| 292-300 | GVFRGIQDV | 124 | A0201, A0205, A3, |
| | | | A68.1, A1101, B3901, |
| | | | B5102, B5201, Cw0602 |
| 293-301 | VFRGIQDVR | 125 | A3101 |
| 294-302 | FRGIQDVRR | 126 | B2705 |
| 295-303 | RGIQDVRRV | 127 | B61, B5101, B5102, |
| | | | B5201, Cw0602 |
| 298-306 | QDVRRVPGV | 128 | B61, B3701 |
| 299-307 | DVRRVPGVA | 129 | A68.1, A3302, B7, B8 |

TABLE 30

| | Amino acid | SEQ ID | |
|---|---|---|---|
| Position | sequence | NO: | HLA subtype |
| 301-309 | RRVPGVAPT | 130 | B14, B2702, B2705, |
| | | | Cw0301 |
| 302-310 | RVPGVAPTL | 7 | A0205, A24, B7B3701, |
| | | | B3801, B3901, B3902 |
| 303-311 | VPGVAPTLV | 131 | B7, B3501, B5102, |
| | | | B5201, Cw0401 |
| 306-314 | VAPTLVRSA | 132 | B4403 |
| 309-317 | TLVRSASET | 133 | A0201, A0205 |

TABLE 31

| | Amino acid | SEQ ID | |
|---|---|---|---|
| Position | sequence | NO: | HLA subtype |
| 312-320 | RSASETSEK | 134 | A68.1, A1101 |
| 313-321 | SASETSEKR | 135 | A3101, A3302 |
| 315-323 | SETSEKRPF | 136 | B40, B3701, B4403 |
| 316-324 | ETSEKRPFM | 137 | B8, B3501 |
| 317-325 | TSEKRPFMC | 138 | A1, B5801 |
| 318-326 | SEKRPFMCA | 139 | B40, B60, 361, 34403 |
| 319-327 | EKRPFMCAY | 140 | Cw0602, Cw0702 |

TABLE 32

| | Amino acid | SEQ ID | |
|---|---|---|---|
| Position | sequence | NO: | HLA subtype |
| 324-332 | MCAYPGCNK | 141 | A68.1, A1101 |
| 325-333 | CAYPGCNKR | 142 | A1, A68.1, A1101, |
| | | | A3101, A3302 |
| 326-334 | AYPGCNKRY | 143 | A24, Cw0401, Cw0702 |
| 327-335 | YPGCNKRYF | 144 | B3501, B3801, B5201, |
| | | | Cw0401, Cw0702 |
| 329-337 | GCNKRYFKL | 145 | A24, B14, B60, B7, |
| | | | B8, B3902, Cw0301 |

TABLE 33

| | Amino acid | SEQ ID | |
|---|---|---|---|
| Position | sequence | NO: | HLA subtype |
| 332-340 | KRYFKLSHL | 146 | B14, B8, B2702, |
| | | | B2705, B3901, B3902, |
| | | | Cw0301, Cw0602 |
| 334-342 | YFKLSHLQM | 147 | Cw0401 |
| 337-345 | LSHLQMHSR | 148 | A68.1, A3302 |
| 340-348 | LQMHSRKHT | 149 | A0201, A0205 |

TABLE 34

| | Amino acid | SEQ ID | |
|---|---|---|---|
| Position | sequence | NO: | HLA subtype |
| 343-351 | HSRKHTGEK | 150 | A68.1 |
| 345-353 | RKHTGEKPY | 151 | Cw0702 |
| 347-355 | HTGEKPYQC | 152 | B8, B5801 |
| 349-357 | GEKPYQCDF | 153 | B40, B3701, B4403 |

TABLE 35

| | Amino acid | SEQ ID | |
|---|---|---|---|
| Position | sequence | NO: | HLA subtype |
| 351-359 | KPYQCDFKD | 154 | B5102 |
| 354-362 | QCDFKDCER | 155 | A1, A68.1, A3101, |
| | | | A3302 |
| 356-364 | DFKDCERRF | 156 | A24, Cw0401 |
| 358-366 | KDCERRFSR | 157 | A3101 |

TABLE 36

| | | | |
|---|---|---|---|
| | Amino acid | SEQ ID | |
| Position | sequence | NO: | HLA subtype |

| | | | |
|---|---|---|---|
| 362-370 | RRFSRSDQL | 158 | B2702, B2705, B3901, |
| | | | Cw0301, Cw0602 |
| 363-371 | RFSRSDQLK | 159 | A1101 |
| 364-372 | FSRSDQLKR | 160 | A68.1, A3302 |
| 366-374 | RSDQLKRHQ | 161 | A1 |
| 368-376 | DQLKRHQRR | 162 | A68.1, A3101, A3302 |

TABLE 37

| | Amino acid | SEQ ID | |
|---|---|---|---|
| Position | sequence | NO: | HLA subtype |
| 371-379 | KRHQRRHTG | 163 | B14 |
| 372-380 | RHQRRHTGV | 164 | B14, B3801, B3901 |
| 373-381 | HQRRHTGVK | 165 | A1101, A3101, B2705 |
| 375-383 | RRHTGVKPF | 166 | B2702, B2705, Cw0102 |
| 379-387 | GVKPFQCKT | 167 | A68.1 |

TABLE 38

| | Amino acid | SEQ ID | |
|---|---|---|---|
| Position | sequence | NO: | HLA subtype |
| 383-391 | FQCKTCQRK | 168 | A3, A1101, A3101, |
| | | | B2705 |
| 384-392 | QCKTCQRKF | 169 | B62, B8 |
| 386-394 | KTCQRKFSR | 170 | A1, A3, A68.1, |
| | | | A1101, A3101 |
| 387-395 | TCQRKFSRS | 171 | B8 |
| 389-397 | QRKFSRSDH | 172 | B2702, B2705 |
| 390-398 | RKFSRSDHL | 173 | B14, B3901, 83902, |
| | | | Cw0301 |

TABLE 39

| | Amino acid | SEQ ID | |
|---|---|---|---|
| Position | sequence | NO: | HLA subtype |
| 391-399 | KFSRSDHLK | 174 | A1101, A3101 |
| 394-402 | RSDHLKTHT | 175 | A1, B5801 |
| 396-404 | DHLKTHTRT | 176 | B3801, B3901 |

TABLE 40

| | Amino acid | SEQ ID | |
|---|---|---|---|
| Position | sequence | NO: | HLA subtype |
| 401-409 | HTRTHTGKT | 177 | B8 |
| 406-414 | TGKTSEKPF | 178 | B5201 |
| 408-416 | KTSEKPFSC | 179 | A0201, B5801 |
| 409-417 | TSEKPFSCR | 180 | A1, A68.1, A3302 |
| 410-418 | SEKPFSCRW | 181 | B40, B4403 |

TABLE 41

| | Amino acid | SEQ ID | |
|---|---|---|---|
| Position | sequence | NO: | HLA subtype |
| 412-420 | KPFSCRWPS | 182 | B3501, B5102 |
| 415-423 | SCRWPSCQK | 183 | A1101 |
| 416-424 | CRWPSCQKK | 184 | B2702, B2705 |
| 417-425 | RWPSCQKKF | 185 | A24, B3801, Cw0401 |
| 418-426 | WPSCQKKFA | 186 | B5102 |
| 419-427 | PSCQKKFAR | 187 | A3302 |
| 420-428 | SCQKKFARS | 188 | B8 |

TABLE 42

| | Amino acid | SEQ ID | |
|---|---|---|---|
| Position | sequence | NO: | HLA subtype |
| 423-431 | KKFARSDEL | 189 | B14, B3901, B3902, |
| | | | Cw0301, Cw0602 |
| 424-432 | KFARSDELV | 190 | Cw0401 |
| 425-433 | FARSDELVR | 191 | A68.1, A1101, A3101, |
| | | | A3302 |
| 426-434 | ARSDELVRH | 192 | B2702, B2705 |
| 427-435 | RSDELVRHH | 193 | A1 |
| 428-436 | SDELVRHHN | 194 | B3701 |
| 429-437 | DELVRHHNM | 195 | B14, B40, B60, B61, |
| | | | B3701, B4403, Cw0301 |

TABLE 43

| | Amino acid | SEQ ID | |
|---|---|---|---|
| Position | sequence | NO: | HLA subtype |
| 432-440 | VRHHNMHQR | 196 | B2705 |
| 433-441 | RHHNMHQRN | 197 | B3801 |
| 434-442 | HHNMHQRNM | 198 | B3901 |
| 436-444 | NMHQRNMTK | 199 | A3, A1101, A3101 |
| 437-445 | MHQRNMTKL | 200 | B14, B3701, B3901, |
| | | | B3902, Cw0301 |
| 439-447 | QRNMTKLQL | 201 | B14, B2702, B2705, |
| | | | B3901, Cw0602 |
| 440-448 | RNMTKLQLA | 202 | B61 |

TABLE 44

| | Amino acid | SEQ ID | |
|---|---|---|---|
| Position | sequence | NO: | HLA subtype |
| 441-449 | NMTKLQLAL | 203 | A0201, A0205, A24, |
| | | | A3, B7, B3902, |
| | | | Cw0602 |

The “MHC class I-restricted WT1 peptide” may preferably be a peptide comprising an amino acid sequence selected from:

(SEQ ID NO: 2)
RMFPNAPYL,
(SEQ ID NO: 3)
CMTWNQMNL,
(SEQ ID NO: 5)
ALLPAVPSL,
(SEQ ID NO: 6)
SLGEQQYSV,
and
(SEQ ID NO: 7)
RVPGVAPTL

or a peptide comprising an amino acid sequence that differs from the amino acid sequence selected from SEQ ID NOS: 2, 3, 5, 6, and 7 by alteration of one or several amino acid residues. A peptide consisting of an amino acid sequence selected from SEQ ID NOS: 2, 3, 5, 6, and 7 is also preferable.

Examples of the altered killer peptide include the following peptides:

(SEQ ID NO: 2)
as an altered killer peptide of RMFPNAPYL,
(SEQ ID NO: 204)
RYFPNAPYL (WO03/106682),
(SEQ ID NO: 205)
FMFPNAPYL,
(SEQ ID NO: 206)
RLFPNAPYL,
(SEQ ID NO: 207)
RMMPNAPYL,
(SEQ ID NO: 208)
RMFPNAPYV
or
(SEQ ID NO: 209)
YMFPNAPYL (WO2009/072610);
(SEQ ID NO: 3)
as an altered killer peptide of CMTWNQMNL,
(SEQ ID NO: 4)
CYTWNQMNL (WO02/79253),
(SEQ ID NO: 210)
Xaa-Met-Thr-Trp-Asn-Gln-Met-Asn-Leu
(wherein Xaa is Ser or Ala)
or
(SEQ ID NO: 211)
Xaa-Tyr-Thr-Trp-Asn-Gln-Met-Asn-Leu
(wherein Xaa is Ser, Ala, Abu, Arg, Lys, Orn, Cit,
Leu, Phe or Asn) (WO2004/026897);
(SEQ ID NO: 5)
as an altered killer peptide of ALLPAVPSL,
(SEQ ID NO: 212)
AYLPAVPSL (WO2003/106682);
(SEQ ID NO: 6)
as an altered killer peptide of SLGEQQYSV,
(SEQ ID NO: 213)
FLGFQQYSV,
(SEQ ID NO: 214)
SMGEQQYSV,
or
(SEQ ID NO: 215)
SLMEQQYSV (WO 2009/072610);
and
(SEQ ID NO: 7)
as an altered killer peptide of RVPGVAPTL,
(SEQ ID NO: 216)
RYPGVAPTL (WO2003/106682).

The term “MHC class II-restricted” means the ability of a peptide to bind to an MHC class II molecule and induce helper T cells.

HLA corresponding to MHC class II-molecules has subtypes including HLA-DR, DQ and DP subtypes. Restriction to HLA-DR, HLA-DQ, or HLA-DP is preferred as the restriction to MHC class II of an “MHC class II-restricted” peptide. Restriction to a subtype selected from the followings is more preferred: DRB1*0101, DRB1*0405, DRB1*0802, DRB1*0803, DRB1*0901, DRB1*1201, DRB1*1403, DRB1*1501, DRB1*1502, DPB1*0201, DPB1*0202, DPB1*0402, DPB1*0501, DPB1*0901, DQB1*0301, DQB1*0302, DQB1*0401, DQB1*0501, DQB1*0601, DQB1*0602 and DRB5*0102. Restriction to a subtype selected from DRB1*0101, DRB1*0405, DRB1*1502, DPB1*0201, DPB1*0202 and DQB10601 is most preferred.

The term “MHC class II-restricted peptide” as used herein refers to a peptide being capable of binding to an MHC class II molecule and inducing helper T cells in vitro and/or in vivo (in other word, a peptide having an ability to induce helper T cells.). An “MHC class II-restricted WT1 peptide” is a WT1 peptide being an “MHC class II-restricted peptide”. The “MHC class II-restricted peptide” and “MHC class II-restricted WT1 peptide” are often herein referred to as “helper peptide” and “WT1 helper peptide”, respectively. The “MHC class II-restricted WT1 peptide” may consists of a sequence of any number of amino acids of any type, so long as it functions as the “MHC class II-restricted WT1 peptide” as defined above. However, the longer a peptide chain is, the more susceptible it may be to degradation by a proteolytic enzyme. Also, too small peptide may not successfully be caught in a peptide-binding groove of an MHC class II molecule. The “MHC class II-restricted WT1 peptide” typically consists of 9 to 30 amino acid residues, preferably 10 to 25 amino acid residues, more preferably 12 to 24 amino acid residues, or still more preferably 15 or 22 amino acid residues.

Examples of the “MHC class II-restricted WT1 peptide” include a peptide comprising an amino acid sequences selected from the following amino acid sequences:

(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG
and
(SEQ ID NO: 241)
FRGIQDVRRVSGVAPTLVR

or a peptide comprising an amino acid sequence that differs from the amino acid sequence selected from SEQ ID NOS: 217-241 by alteration of one or several amino acid residues and having an ability to induce helper T cells.

As filed in the PCT application No. PCT/JP2017/042760, the inventors have found that some WT1 helper peptides commonly have a motif: KLSHL from the amino acid sequence of WT1 protein. The motif: KLSHL corresponds to the amino acid sequence at positions 336-340 of SEQ ID NO: 1. Thus, in an embodiment, an “MHC class II-restricted WT1 peptide” is a partial peptide of WT1 protein consisting of an amino acid sequence of 9 to 30 amino acid residues including the motif: KLSHL as part thereof, or a peptide consisting of an amino acid sequence that differs from the amino acid sequence of the partial peptide by alteration of one or several amino acid residues and has an ability to induce helper T cells.

In a further embodiment, an “MHC class II-restricted WT1 peptide” is a partial peptide of WT1 protein consisting of an amino acid sequence of 10 to 25 amino acid residues, 12 to 24 amino acid residues, or 15 to 22 amino acid residues including the motif: KLSHL as part thereof, or a peptide consisting of an amino acid sequence that differs from the amino acid sequence of the partial peptide by alteration of one or several amino acid residues and has an ability to induce helper T cells.

As defined above, the term “peptide comprising an amino acid sequence” refers to a peptide having a given amino acid sequence, which may, as usually understood, optionally have extra sequence(s) of amino acid residue(s) attached to the N-terminal and/or C-terminal amino acid of the given sequence. Therefore, when a partial peptide of WT1 protein consists of an amino acid sequence of 9 to 30 amino acid residues, 10 to 25 amino acid residues, 12 to 24 amino acid residues, or 15 to 22 amino acid residues including the motif: KLSHL as part thereof, the partial peptide of WT1 protein is a peptide corresponding to part of the amino acid sequence of SEQ ID NO: 1 consisting of the motif: KLSHL and sequence(s) of necessary number of amino acid residues directly attached to the N-terminus and/or C-terminus of the motif. Specifically, such a partial peptide corresponds to an amino acid sequence consisting of contiguous 9 to 30 amino acids, 10 to 25 amino acids, 12 to 24 amino acids, or 15 to 22 amino acids including the motif: KLSHL from SEQ ID NO: 272, which corresponds to the sequence of amino acids 311-365 in SEQ ID NO: 1.

The amino acid sequence at positions 311-365 of SEQ ID NO: 1 (corresponding to the amino acid sequence of SEQ ID NO: 272) is as follows:

```
Val  Arg  Ser  Ala  Ser  Glu  Thr  Ser  Glu  Lys  Arg  Pro  Phe  Met  Cys  Ala  Tyr  Pro  Gly  Cys
                  315 320 325 330

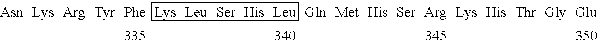

Lys  Pro  Tyr  Gln  Cys  Asp  Phe  Lys  Asp  Cys  Glu  Arg  Arg  Phe  Ser
                  355  360 365
```

Examples of the “MHC class II-restricted WT1 peptide” including the motif: KLSHL as part thereof include a peptide comprising an amino acid sequence selected from:

(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
(SEQ ID NO 248)
RYFKLSHLQMHSRKHTGE,
(SEQ ID NO: 249)
YFKLSHLQMHSRK,
(SEQ ID NO: 250)
FKLSHLQMHSRK,
(SEQ ID NO: 251)
KLSHLQMHSRK,
(SEQ ID NO: 252)
FKLSHLQMHSRKHTGE,
(SEQ ID NO: 253)
KLSHLQMHSRKH,
(SEQ ID NO: 254)
YPGCNKRYFKLSHLQMHSRK,
(SEQ ID NO: 255)
AYPGCNKRYFKLSHLQMHSR,
(SEQ ID NO: 256)
AYPGCNKRYFKLSHLQMHS,
(SEQ ID NO: 257)
AYPGCNKRYFKLSHLQM,
(SEQ ID NO: 258)
AYPGCNKRYFKLSHLQ,
(SEQ ID NO: 259)
YFKLSHLQMHSRKHTGE,
(SEQ ID NO: 260)
RYFKLSHLQMHSRKHTG,
(SEQ ID NO: 261)
RYFKLSHLQMHSR,
(SEQ ID NO: 262)
RYFKLSHLQMHS,
(SEQ ID NO: 263)
RYFKLSHLQM,
(SEQ ID NO: 264)
YPGCNKRYFKLSHL,
(SEQ ID NO: 265)
PGCNKRYFKLSHL,
(SEQ ID NO: 266)
CNKRYFKLSHL,
(SEQ ID NO: 267)
NKRYFKLSHL,
(SEQ ID NO: 268)
KLSHLQMHSRKHTG,
(SEQ ID NO: 269)
KLSHLQMHSRKHT,
(SEQ ID NO: 270)
KLSHLQMHSRK,
and
(SEQ ID NO: 271)
KLSHLQMHSR

or a peptide comprising an amino acid sequence that differs from the amino acid sequence selected from SEQ ID NOS: 221-236 and 242-2/1 by alteration of one or several amino acid residues and having an ability to induce helper T cells.

Examples of the “MHC class II-restricted WT1 peptide containing at least one cysteine residue” include a peptide comprising an amino acid sequence containing at least one cysteine residue selected from:

(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
(SEQ ID NO: 248)
RYFKLSHLQMHSRKHTGE,
(SEQ ID NO: 249)
YFKLSHLQMHSRK,
(SEQ ID NO: 250)
FKLSHLQMHSRK,
(SEQ ID NO: 251)
KLSHLQMHSRK,
(SEQ ID NO: 252)
FKLSHLQMHSRKHTGE,
(SEQ ID NO: 253)
KLSHLQMHSRKH,
(SEQ ID NO: 254)
YPGCNKRYFKLSHLQMHSRK,
(SEQ ID NO: 255)
AYPGCNKRYFKLSHLQMHSR,
(SEQ ID NO: 256)
AYPGCNKRYFKLSHLQMHS,
(SEQ ID NO: 257)
AYPGCNKRYFKLSHLQM,
(SEQ ID NO: 258)
AYPGCNKRYFKLSHLQ,
(SEQ ID NO: 259)
YFKLSHLQMHSRKHTGE,
(SEQ ID NO: 260)
RYFKLSHLQMHSRKHTG,
(SEQ ID NO: 261)
RYFKLSHLQMHSR,
(SEQ ID NO: 262)
RYFKLSHLQMHS,
(SEQ ID NO: 263)
RYFKLSHLQM,
(SEQ ID NO: 264)
YPGCNKRYFKLSHL,
(SEQ ID NO: 265)
PGCNKRYFKLSHL,
(SEQ ID NO: 266)
CNKRYFKLSHL,
(SEQ ID NO: 267)
NKRYFKLSHL,
(SEQ ID NO: 268)
KLSHLQMHSRKHTG,
(SEQ ID NO: 269)
KLSHLQMHSRKHT,
(SEQ ID NO: 270)
KLSHLQMHSRK,
and
(SEQ ID NO: 271)
KLSHLQMHSR

or a peptide comprising an amino acid sequence containing at least one cysteine residue that differs from the amino acid sequence selected from SEQ ID NOS: 217-271 by alteration of one or several amino acid residues and having an ability to induce helper T cells.

Preferred examples of the “MHC class II-restricted WT1 peptide containing at least one cysteine residue” include a peptide comprising an amino acid sequence containing at least one cysteine residue selected from:

(SEQ ID NO: 221)
AYPGCNKRYFKLSHL,
(SEQ ID NO: 222)
YPGCNKRYFKLSHLQ,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 238)
CWAPVLDFAPPGASAYGSL,
(SEQ ID NO: 239)
WAPVLDFAPPGASAYGSLC,
(SEQ ID NO: 243)
AYPGCNKRYFKLSHLQMH,
(SEQ ID NO: 244)
AYPGCNKRYFKLSHLQMHSRK,
and
(SEQ ID NO: 246)
GCNKRYFKLSHL.

An MHC class II-restricted peptide may complex with an MHC class II molecule in any of HLA-DR, HLA-DQ or HLA-DP subclass. In a preferred embodiment, an MHC class II-restricted peptide induces helper T cells by binding to an MHC class II molecule selected from DRB1*0101, DRB1*0405, DRB1*0802, DRB1*0803, DRB1*0901, DRB1*1201, DRB1*1403, DRB1*1501, DRB1*1502, DPB1*0201, DPB1*0202, DPB1*0402, DPB1*0501, DPB1*0901, DQB1*0301, DQB1*0302, DQB1*0401, DQB1*0501, DQB1*0601, DQB1*0602 and DRB5*0102. More preferably, an MHC class II-restricted peptide induces helper T cells by binding to an MHC class II molecule selected from DRB1*0101, DRB1*0405, DRB1*1403, DRB1*1502, DPB1*0201, DPB1*0202, DPB1*0901, DQB1*0301, DQB1*0601 and DRB5*0102. Most preferably, an MHC class II-restricted peptide induces helper T cells by binding to an MHC class II molecule selected from DRB1*0101, DRB1*0405, DRB1*1502, DPB1*0201, DPB1*0202 and DQB1*0601.

A helper peptide consisting of an altered amino acid sequence is also herein referred to as an “altered helper peptide”. If a partial peptide of WT1 protein useful as a WT1 helper peptide consisting of an amino acid sequence of 9 to 30 amino acid residues including the sequence: KLSHL is altered, the altered peptide preferably has modification in amino acid residue(s) out of the sequence: KLSHL. An “MHC class II-restricted WT1 peptide” as “cancer antigen peptide B” may have an extra sequence of amino acid residue (s) attached preferably to its C-terminus. An “MHC class II-restricted WT1 peptide” as “cancer antigen peptide C” may have an extra sequence of amino acid residue(s) attached preferably to its N-terminus.

Amino acid substitution may be made at any of amino acid residues constituting a binding motif to an HLA antigen. When a helper peptide consisting of an amino acid sequence of nine amino acid residues including a binding motif to HLA-DRB1*0405 is altered by amino acid substitution, the substitution may preferably be made at positions 1 (N-terminus), 4, 6 and/or 9 (C-terminus). In a preferred embodiment, a helper peptide consisting of a sequence of nine amino acid residues including a binding motif to HLA-DRB1*0405 may be altered by amino acid substitution to have an amino acid residue(s) selected from:

phenylalanine, tryptophan, valine, isoleucine, leucine or methionine at position 1 (N-terminus);
valine, isoleucine, methionine, aspartic acid and glutamic acid at position 4;
asparagine, serine, threonine, glutamine, lysine and aspartic acid at position 6; and/or
aspartic acid, glutamic acid and glutamine at position 9 (C-terminus).

A peptide may have modification in amino acid residue(s) in its sequence. Modification may be made by a conventional way, for example by esterification, alkylation, halogenation, phosphorylation, sulfonation, or amidation on a functional group in an amino acid residue. Amino acid modification in a peptide can also be addition of any of various moieties to N-terminus and/or C-terminus of a peptide. A peptide may be modified by addition of such a moiety that would modulate solubility of the peptide, stabilize the peptide against, for example, proteolytic degradation, direct the peptide to a specific tissue or organ, or improve capturing of the peptide by antigen presenting cells.

In a peptide, an amino group of its N-terminal amino acid or a carboxyl group of its C-terminal amino acid may be modified. The amino group may be modified, for example by addition of one to three groups selected from C₁₋₆-alkyl, phenyl, cycloalkyl, or acyl such as C₁₋₆-alkanoyl, phenyl-C₁₋₆-alkanoyl, C₅₋₇-cycloalkyl-carbonyl, C₁₋₆-alkylsulfonyl, phenylsulfonyl, C₂₋₆-alkoxy-carbonyl, phenyl-alkoxycarbonyl, C₅₋₇-cycloalkoxy-carbonyl, or phenoxycarbonyl. The carboxyl group of the C-terminal amino acid may be modified, for example by conversion to an ester, such as a C₁₋₆-alkyl ester, a phenyl-C₀₋₆-alkyl ester, or a C₅₋₇-cycloalkyl ester, or to an amide such as an unsubstituted amide, a mono- or di-C₁₋₆-alkylamide, a mono- or di-phenyl-C₀₋₆-alkylamide, or a di-substituted amide wherein the two substituents forms together with the nitrogen atom they attach to a 5- to 7-membered azacycloalkane.

In a peptide, a bond between amino acid residues may be peptide bond or other type of bond such as carbon-carbon bond, carbon-nitrogen bond, or carbon-sulfur bond. A peptide as described herein may comprise one or more D-amino acid residues.

The above description about modification in a peptide is illustrative only, and variations thereof would be conceivable to a person skilled in the art. Such a modified peptide would be prepared, tested or used by an ordinarily skilled person in the art.

Examples of the compound of formula (1) include a compound of formula (4):

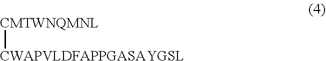

wherein C—C shown in the formula means that the C residues are linked together by a disulfide bond; a compound of formula (5):

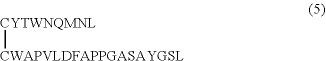

wherein C—C shown in the formula means that the C residues are linked together by a disulfide bond; a compound of formula (6):

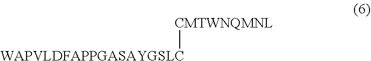

wherein C—C shown in the formula means that the C residues are linked together by a disulfide bond; and a compound of formula (7):

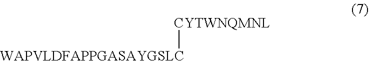

wherein C—C shown in the formula means that the C residues are linked together by a disulfide bond.

The ability of a peptide to induce CTLs or helper T cells can be confirmed by a conventional method. Induction of CTLs can be confirmed, for example by counting CTLs by HLA tetramer method (Int. J. Cancer: 100, 565-570 (2002)) or limiting dilution method (Nat. Med.: 4, 321-327 (1998)). Induction of CTLs by an HLA-A24-restricted peptide may also be confirmed by using an HLA-A24 mouse model as described in WO 02/47474 or Int. J. Cancer: 100, 565-570 (2002). Induction of helper T cells can be confirmed, for example by a method as described in Cancer Immunol. Immunother. 51: 271 (2002) or in the Example section herein.

A peptide or compound as described herein, or an intermediate peptide for the synthesis thereof may be synthesized in accordance with a method described in the Example section herein or by using a conventional technique for peptide synthesis as described, for example, in Peptide Synthesis, Interscience, New York, 1966; The Proteins, Vol 2, Academic Press Inc., New York, 1976; peptide synthesis, Maruzen Co., LTD., 1975; Basics and Experiment of Peptide Synthesis, Maruzen Co., LTD., 1985; or Development of Pharmaceutical Product subsequent vol. 14, Peptide Synthesis, Hirokawa Shoten, 1991. Examples of such a technique include solid phase synthesis by Fmoc method or Boc method, or liquid phase synthesis by sequential condensation of Boc-amino acid or Z-amino acid in a liquid phase (wherein Fmoc means 9-fluorenylmethoxycarbonyl, Boc means t-butoxycarbonyl, and Z means benzyloxycarbonyl). The peptide may be obtained by a genetic technique by using a nucleotide sequence encoding the peptide in accordance with a conventionally known method as described, for example, in Molecular Cloning, T. Maniatis et al., CSH Laboratory (1983), DNA Cloning, DM. Glover, IRL PRESS (1985).

In the course of the synthesis of a compound of formula (1), a functional group on an intermediate compound, such as amino, carboxyl or mercapto may be protected with an appropriate protecting group, or deprotected as needed by a conventional technique. For information about such a protecting group, or a protection or deprotection method, “Protective Groups in Organic Synthesis 2nd Edition (John Wiley & Sons, Inc.; 1990)” may be referred to. As a protecting group for mercapto, an acetamidomethyl group or a trityl group may be useful.

When a compound of formula (1) includes a disulfide bond, the linkage is formed between two different, cysteine-comprising peptide components in the compound, or between a cysteine-comprising peptide component and a cysteine residue in the compound. Such a disulfide bond can be formed by a method as described, for example, in Peptide Synthesis, Interscience, New York, 1966; The Proteins, Vol. 2, Academic Press Inc., New York, 1976; peptide synthesis, Maruzen Co., LTD., 1975; Basics and Experiment of peptide synthesis, Maruzen Co., LTD., 1985; or Development of Pharmaceutical Product sequential vol. 14, Peptide Synthesis, Hirokawa Shoten, 1991.

Specifically, for preparing a compound having a disulfide bond (a disulfide compound) from a peptide having one cysteine residue, the peptide may be subjected to a deprotection reaction for removal of any protecting groups on functional groups including mercapto on the cysteine residue, and then treated in an inert solvent under an oxidative condition for forming a disulfide bond. A disulfide compound may also be prepared from two different, mercapto-having intermediates by treating them in an appropriate solvent under an oxidative condition. Oxidative conditions for disulfide bond formation are known in the field of peptide synthesis. For example, a known method of iodine oxidation, air oxidation under an alkaline condition, or oxidation by an oxidizing agent under an alkaline or acidic condition may be used for forming a disulfide bond. As an oxidizing agent, iodine, dimethyl sulfoxide (DMSO), or potassium ferricyanide may be used. As a solvent for the reaction, water, acetic acid, methanol, chloroform, DMF, or DMSO, or a mixture thereof may be used. Such an oxidative condition often gives a product in the form of a mixture of symmetric and asymmetric disulfide compounds. A desired asymmetric disulfide compound may be recovered or purified by an appropriate chromatographic method or recrystallization. An intermediate having an activated mercapto group, for example a mercapto group bound to an Npys group (3-nitro-2-pyridinesulphenyl group) may be used. For forming a disulfide bond on a given mercapto group on an intermediate, the group may be activated by 2,2′-dithiobis(5-nitropyridine) in advance of coupling with another intermediate (Tetrahedron Letters. Vol. 37. No. 9, pp. 1347-1350).

The methods as described above may be useful for preparing a disulfide compound from a peptide having more than one cysteine residue. In that case, however, a mixture of different disulfide compounds having a disulfide bond between different cysteine residues may be formed. For selectively preparing a product dimerized by a disulfide bond between specific positions of monomers, different protecting groups can be used in combination for protection of functional groups on the cysteine residues. Examples of such combination of protecting groups include a combination of MeBzl (methylbenzyl) and Acm (acetamidomethyl); Trt (trityl) and Acm; Npys (3-nitro-2-pyridylthio) and Acm; and S-Bu-t (S-tert-butyl) and Acm. For example, when a peptide protected with a combination of MeBzi and Acm is used for selective disulfide bond formation, all the MeBzl protecting groups, and the Acm protecting groups on functional groups on amino acid residues other than certain cysteine residues may be removed in the first step. Then, by treating the peptide monomer in a solution under air oxidation condition, a disulfide bond can be formed between selectively deprotected cysteine residues of the monomers. Then, through removal of remaining Acm protecting groups and treatment under oxidative condition with iodine, a further disulfide bond can be formed on the newly deprotected cysteine residues.

The peptide, compound or intermediate synthesized may be purified by any purification method know in the art or in the field of peptide chemistry. Examples of such a purification technique include various types of chromatography (e.g., silica gel column chromatography, ion exchange column chromatography, gel filtration or reversed-phase chromatography), and recrystallization from a solvent, for example an alcohol, such as methanol, ethanol or 2-propanol; an ether, such as diethyl ether; an ester, such as ethyl acetate; an aromatic hydrocarbon, such as benzene or toluene; a ketone, such as acetone; a hydrocarbon, such as hexane; an aprotic solvent, such as dimethylformamide or acetonitrile; water; or a mixture thereof. For further useful purification methods, reference can be made, for example, to Jikken Kagaku Kouza (The Chemical Society of Japan ed., Maruzen) vol. 1. Purification methods for disulfide compounds are also described in Peptide Synthesis, Interscience, New York, 1966; The Proteins, Vol. 2, Academic Press Inc., New York, 1976; peptide synthesis, Maruzen Co., LTD., 1975; Basics and Experiment of peptide synthesis, Maruzen Co., LTD., 1985; or Development of Pharmaceutical Product sequential vol. 14, Peptide Synthesis, Hirokawa Shoten, 1991. Purification by HPLC is preferred.

A compound of formula (1) may have one or more asymmetric centers. Such a compound can be prepared from a starting material (an amino acid) having corresponding asymmetric centers. Also, a compound of formula (1) can be obtained in a high optical purity by inclusion of an optical resolution step in a process for its synthesis. For example, in accordance with a diastereomer method for optical resolution, a compound of formula (1) or an intermediate product can be treated with an optically active acid (e.g., a monocarboxylic acid such as mandelic acid, N-benzyloxyalanine, or lactic acid, a dicarboxylic acid such as tartaric acid, o-diisopropylidenetartaric acid, or malic acid, or a sulfonic acid such as camphorsulfonic acid or bromocamphorsulfonic acid) in an inert solvent (e.g., an alcohol such as methanol, ethanol, or 2-propanol, an ether such as diethyl ether, an ester such as ethyl acetate, a hydrocarbon such as toluene, an aprotic solvent such as acetonitrile, or a mixture thereof) to form salts. For optically resolving a compound of formula (1) or an intermediate having an acidic functional group such as carboxyl, its salts can be formed with an optically active amine (e.g., an organic amine such as α-phenethylamine, kinin, quinidine, cinchonidine, cinchonine, or strychnine).

The salts may be formed at a temperature in the range from room temperature up to the boiling point of a solvent used. For obtaining a product in a highly optically pure form, it may be desirable to raise the temperature to around the boiling point of the solvent for a period of time. Salts formed are crystallized, and then filtered, optionally with cooling for an improved yield. An optically active acid or amine used for the salt formation may be used in an amount of about 0.5 to about 2.0 equivalents, preferably about 1 equivalent, relative to the amount of a compound to optically resolve. A crystalline product may optionally be further purified by recrystallization from an inert solvent (e.g., an alcohol such as methanol, ethanol, or 2-propanol, an ether such as diethyl ether, an ester such as ethyl acetate, a hydrocarbon solvent such as toluene, an aprotic solvent such as acetonitrile, or a mixture thereof). A product recovered in the form of a salt may optionally be converted to a free base or acid by treatment with an acid or base.

The term “pharmaceutically acceptable salt” as used herein includes an acid addition salt and a base addition salt. The acid addition salt may be an inorganic acid salt, such as hydrochloride, hydrobromide, sulfate, hydroiodide, nitrate, or phosphate, or an organic acid salt such as citrate, oxalate, acetate, formate, propionate, benzoate, trifluoroacetate, maleate, tartrate, methanesulfonate, benzenesulfonate, or p-toluenesulfonate. The base addition salt may be a salt with an inorganic base, such as sodium salt, potassium salt, calcium salt, magnesium salt, or ammonium salt, a salt with an organic base, such as triethylammonium salt, triethanolammonium salt, pyridinium salt, or diisopropylammonium salt. The “pharmaceutically acceptable salt” also includes a salt with a basic or acidic amino acid, such as arginine, aspartic acid, or glutamic acid. The term “peptide” or “compound” used herein includes a peptide or compound in the form of a pharmaceutically acceptable salt, unless the context requires otherwise.

The present disclosure further includes a hydrate or a solvate such as an ethanol solvate of the compound of formula (1) or the peptide or or a pharmaceutically acceptable salt thereof as described herein. The present disclosure also includes any stereoisomer such as a diastereomer or an enantiomer, and any crystalline form of the compound or the peptide as described herein.

The compound of formula (1) or a pharmaceutically acceptable salt thereof as described herein is useful for treating or preventing (including prevention of recurrence) a cancer accompanied by WT1 gene expression or a cancer accompanied by an elevated level of WT1 gene expression. For example, the compound of formula (1) or a pharmaceutically acceptable salt thereof can be an active ingredient of a pharmaceutical composition (for example, a cancer vaccine), or a composition for inducing CTLs in cellular immunotherapy for a cancer.

The compound of formula (1) or a pharmaceutically acceptable salt thereof as described herein may be used for treating or preventing (including prevention of recurrence) “a cancer accompanied by WT1 gene expression” or “a cancer accompanied by an elevated level of WT1 gene expression”. Examples of such a cancer include a hematologic cancer such as leukemia, myelodysplastic syndrome, multiple myeloma, and malignant lymphoma, and a solid cancer such as gastric cancer, colorectal cancer, lung cancer, breast cancer, germ cell cancer, liver cancer, skin cancer, urinary bladder cancer, prostate cancer, uterine cancer, cervical cancer, ovarian cancer, glioblastoma multiforme, malignant melanoma, non-small cell lung cancer, renal cell carcinoma or brain tumor.

Further examples of cancers that can be treated or prevented by the compound of formula (1) or a pharmaceutically acceptable salt thereof include bone cancer, pancreatic cancer, cancer of the head or neck, cutaneous or intraocular malignant melanoma, rectal cancer, cancer of the anal region, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia such as acute myeloid leukemia, chronic myeloid leukemia, acute lymphoblastic leukemia, or chronic lymphocytic leukemia, childhood solid tumor, lymphocytic lymphoma, cancer of the kidney or ureter, carcinoma of the renal pelvis, central nervous system (CNS) tumor, primary CNS lymphoma, tumor angiogenesis, spinal tumor, brainstem glioma, pituitary adenoma, Kaposi's sarcoma, epidermoid cancer, squamous cell cancer, T-cell lymphoma, environmentally induced cancers including asbestos-induced cancer, and combinations of the cancers as described above.

The compound of formula (1) or a pharmaceutically acceptable salt thereof as described herein may be used in combination with at least one different cancer antigen peptide, in particular, an MHC class I-restricted WT1 peptide or an MHC class II-restricted WT1 peptide, a conjugate thereof, or a pharmaceutically acceptable salt thereof. Examples of different cancer antigen peptides or conjugates include peptides or derivatives thereof, or conjugates thereof as described in the following publications: WO 2000/006602, WO 2002/079253, WO 2003/106682, WO 2004/026897, WO 2004/063903, WO 2007/063903, WO 2010/123065, WO 2014/157692, WO 2005/053618, WO 2007/047764, WO 2007/120673, WO 2005/045027, WO 2010037395, WO 2000/018795, WO 2002/028414, WO 2003/037060, and WO 2004/100870.

In an embodiment, the compound of formula (1) or a pharmaceutically acceptable salt thereof is used in combination with at least one cancer antigen peptide E or a pharmaceutically acceptable salt thereof, wherein the at least one cancer antigen peptide E is an MHC class I-restricted peptide, preferably, an MHC class I-restricted WT1 peptide, consisting of 7 to 30 amino acid residues.

The compound of formula (1) or a pharmaceutically acceptable salt thereof, or a combination of the compound of formula (1) or a pharmaceutically acceptable salt thereof and at least one different cancer antigen peptide or a pharmaceutically acceptable salt thereof may be used in combination with at least one different drug (hereinafter referred to as coadministration drug).

The coadministration drug may be an “immunomodulator”. As used herein, the term “immunomodulator” means any agent that controls transmission of a costimulatory signal generated during T cell activation by antigen-presenting cells by interacting with a molecule that is involved in the transmission of the costimulatory signal and present on the antigen-presenting cells and/or T cells, as well as any agent that directly or indirectly controls function of a molecule involved in establishment of immune tolerance (immunosuppression) in the immune system. Since a cancer antigen peptide is effective for increasing tumor-reactive CTLs in a tumor, it is potentially useful as an agent for coadministration with an immunomodulator, for lowering a necessary dose of an immunomodulator or reducing adverse event caused by an immunomodulator. Thus, the present disclosure provides, through the use of a WT1 antigen peptide in combination with an immunomodulator, patients with a therapy having improved efficacy and safety.

The “immunomodulator” can be an agent in the form of an antibody, a nucleic acid, a protein, a peptide, or a small compound, but is not limited thereto. The “antibody” as the “immunomodulator” includes an antibody fragment. Examples of the antibody fragment include heavy and light chain variable regions of an antibody (VH and VL), F(ab′)2, Fab′, Fab, Fv, Fd, sdFv, and scFV. The “protein” as the “immunomodulator” means any protein other than antibodies. Examples of the “immunomodulator” include immune checkpoint inhibitors, costimulatory molecule agonists, immune activating agents, and low-molecular inhibitors.

The “immune checkpoint inhibitor” inhibits immunosuppressive effect induced by cancer cells or antigen presenting cells. Examples of the immune checkpoint inhibitor include, but are not limited to, agents against a molecule selected from the group consisting of: (1) CTLA-4 (e.g., ipilimumab and tremelimumab); (2) PD-1 (e.g., nivolumab, pembrolizumab, AMP-224, AMP-514 (MEDI0680), and pidilizumab (CT-011)); (3) LAG-3 (e.g., IMP-321 and BMS-986016); (4) BTLA; (5) KIR (e.g., IPH2101); (6) TIM-3; (7) PD-L1 (e.g., durvalumab (MEDI4736), MPDL3280A, BMS-936559, and avelumab (MSB0010718C)); (8) PD-L2; (9) B7-H3 (e.g., MGA-271); (10) B7-H4; (11) HVEM; (12) GAL9; (13) CD160; (14) VISTA; (15) BTNL2; (16) TIGIT; (17) PVR; (18) BTN1A1; (19) BTN2A2; (20) BTN3A2 (Nat Rev Drug Discov. 2013; 12: 130-146; Nikkei Medical Cancer Review 2014; 9; Nat Rev Immunol. 2014; 14: 559-69); and (21) CSF1-R.

The “costimulatory molecule agonist” enhances T cell activation by transmission of an auxiliary signal via a costimulatory molecule on the T cells and/or antigen presenting cells, and attenuates the immunosuppressive effect of cancer cells or antigen presenting cells. Examples of the costimulatory molecule agonist include, but are not limited to, agents against a molecule selected from the group consisting of: (1) 4-1BB; (2) 4-1BB-L; (3) OX40 (4) OX40-L; (5) GITR; (6) CD28; (7) CD40; (8) CD40-L; (9) ICOS; (10) ICOS-L; (11) LIGHT; and (12) CD27.

The “immune activating agent” efficiently stimulates killer T cells in the lymph nodes by directly or indirectly activating immune cells such as T cells and dendritic cells. Examples of the immune activating agent include, but are not limited to, Toll-like receptor (TLR) agonists, stimulator of interferon genes (STING) agonists, cytokines, and agents against heat shock protein (HSP).

Examples of the “Toll-like receptor (TLR) agonist” include, but are not limited to, TLR1/2 agonists, TLR2 agonists, TLR3 agonists (e.g., PolyI:C), TLR4 agonists (e.g., S-type lipopolysaccharide, paclitaxel, lipid A, and monophosphoryl lipid A), TLR5 agonists (e.g., flagellin), TLR6/2 agonists (e.g., MALP-2), TLR7 agonist, TLR7/8 agonists (e.g., gardiquimod, imiquimod, loxoribine, and resiquimod (R848)), TLR7/9 agonists (e.g., hydroxychloroquine sulfate), TLR8 agonists (e.g., motolimod (VTX-2337)), TLR9 agonists (e.g., CpG-ODN), and TLR11 agonists (e.g., profilin).

Examples of the “cytokine” include, but are not limited to, IL-1a, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (INF)-α, INF-β, INF-γ, SCF, GM-CSF, G-CSF, M-CSF, erythropoietin, thrombopoietin, macrophage inflammatory protein (MIP), and monocyte chemoattractant protein (MCP).

Examples of the “heat shock protein (HSP)” include, but are not limited to, HSP70, HSP90, HSP90a, HSP900, HSP105, HSP72, and HSP40. Agents against a heat shock protein include HSP inhibitors. Examples of inhibitors to HSP90, for example, include, but are not limited to, tanespimycin (17-AAG), luminespib (AUY-922, NVP-AUY922), alvespimycin (17-DMAG) hydrochloride, ganetespib (STA-9090), BIIB021, onalespib (AT13387), geldanamycin, NVP-BEP800, SNX-2112 (PF-04928473), PF-4929113 (SNX-5422), KW-2478, XL888, VER155008, VER-50589, CH5138303, VER-49009, NMS-E973, PU-H71, HSP990 (NVP-HSP990) and KNK437.

Examples of the “low-molecular inhibitor” include, but are not limited to, histone deacetylase inhibitors, histone demethylase inhibitors, histone acetyltransferase inhibitors, histone methyltransferase inhibitors, DNA methyltransferase inhibitors, anthracycline antibiotics, platinum formulations, MAPK inhibitors, β-catenin inhibitors, STAT3 inhibitors, NF-kB inhibitors, JAK inhibitors, mTOR inhibitors, IDO inhibitors, COX-2 inhibitors, CXCR4 inhibitors, and arginase inhibitors.

Examples of the “histone deacetylase inhibitor” include, but are not limited to, vorinostat (SAHA, MK0683), entinostat (MS-275), panobinostat (LBH589), trichostatin A (TSA), mocetinostat (MGCD0103), BG45, BRD73954, belinostat (PXD101), romidepsin (FK228, depsipeptide), 4SC-202, HPOB, LMK-235, CAY10603, tasquinimod, TMP269, nexturastat A, rocilinostat (ACY-1215), RGFP966, RG2833 (RGFP109), scriptaid, tubastatin A, pracinostat (SB939), CUDC-101, M344, PCI-34051, dacinostat (LAQ824), tubastatin A hydrochloride, abexinostat (PCI-24781), CUDC-907, AR-42, sodium phenylbutyrate, resminostat, tubacin, quisinostat (JNJ-26481585) dihydroch-oride, MC1568, givinostat (ITF2357), droxinostat, chidamide (C S055, HBI-8000), CHR-2485, CHR-3996, DAC-060, FRM-0334 (EVP-0334), MGCD-290, CXD-101 (AZD-9468), CG200745, arginine butyrate, sulforaphane, SHP-141, CUDC-907, YM753 (OBP-801), sodium valproate, apicidin, and CI994 (tacedinaline).

Examples of the “histone demethylase inhibitor” include, but are not limited to, GSK J4 HCl, OG-L002, JIB-04, IOX1, SP2509, ORY-1001 (RG-6016), GSK J1, ML324, and GSK-LSD1 2HCl.

Examples of the “histone acetyltransferase inhibitor” include, but are not limited to, C646, MG149, remodelin, and anacardic acid.

Examples of the “histone methyltransferase inhibitor” include, but are not limited to, pinometostat (EPZ5676), EPZ005678, GSK343, BIX01294, tazemetostat (EPZ6438), 3-deazaneplanocin A (DZNeP) HCl, UNC1999, MM-102, SGC0946, entacapone, EPZ015666, UNC0379, EI1, MI-2 (menin-MLL inhibitor), MI-3 (menin-MLL inhibitor), PFI-2, GSK126, EPZ04777, BRD4770, GSK-2816126, and UNC0631.

Examples of the “DNA methyltransferase inhibitor” include, but are not limited to, decitabine, azatidine, RG108, thioguanine, zebularine, SGI-110, CC-486, SGI-1027, lomeguatrib, and procainamide hydrochloride.

The “anthracycline antibiotic” is intercalated between DNA strands to inhibit DNA relaxation. Examples of the anthracycline antibiotic include, but are not limited to, doxorubicin, liposomal doxorubicin, daunorubicin, pirarubicin, epirubicin, idarubicin, aclarubicin, amrubicin, aloin, and mitoxantrone.

Examples of the “platinum formulation” include, but are not limited to, cisplatin, carboplatin, miboplatin, nedaplatin, satraplatin (JM-126), oxaliplatin (ELOXATIN), triplatin tetranitrate, and DDS formulations thereof.

Examples of the “MAPK inhibitor” include, but are not limited to, SB203580, doramapimod (BIRB796), SB202190 (FHPI), LY2228820, VX-702, SB239063, pexmetinib (ARRY-614), PH-797804, VX-745, and TAK-715.

Examples of the “β-catenin inhibitor” include, but are not limited to, XAV-939, ICG-001, IWR-1-endo, Wnt-C59 (C59), LGK-974, KYO2111, IWP-2, IWP-L6, WIKI4, and FH535.

Examples of the “STAT3 inhibitor” include, but are not limited to, S3I-201, Stattic, niclosamide, nifuroxazide, napabucasin (BBI608), cryptotanshinone, HO-3867, WHI-P154, FLLL32, STA-21, WP1066, and SH-4-54.

Examples of the “NF-kB inhibitor” include, but are not limited to, QNZ (EVP4593), sodium 4-aminosalicylate, JSH-23, phenethyl caffeate, sodium salicylate, andrographolide, and SC75741.

Examples of the “JAK inhibitor” include, but are not limited to, ruxolitinib (INCB018424), tofacitinib (CP-690550) citrate, AZD1480, fedratinib (SAR302503, TG101348), AT9283, tyrphostin B42 (AG-490), momelotinib (CYT387), tofacitinib (CP-690550, tasocitinib), WP1066, TG101209, gandotinib (LY2784544), NVP-BSK805 2HCl, baricitinib (LY3009104, INCB02850), AZ960, CEP-33779, pacritinib (SB1518), WHI-P154, XL019, S-ruxolitinib (INCB018424), ZM39923 HCl, decernotinib (VX-509), cerdulatinib (PRT062070, PRT2070), filgotinib (GLPG0634), FLLL32, peficitinib (ASP015K, JNJ-54781532), GLPG0634 analogue, Go6976, and Curcumol.

Examples of the “mTOR inhibitor” include, but are not limited to, sirolimus (rapamycin), deforolimus (AP23573, MK-8669), everolimus (RAD-001), temsirolimus (CCI-779, NSC683864), zotarolimus (ABT-578), biolimus A9 (umirolimus), AZD8055, KU-0063794, voxtalisib (XL765, SAR245409), MHY1485, dactolisib (BEZ235, NVP-BEZ235), PI-103, and torkinib (PP242).

Examples of the “IDO inhibitor” include, but are not limited to, NLG919, INCB024360 analog, indoximod (NLG-8189), and epacadostat (INCB024360).

Examples of the “COX-2 inhibitor” include, but are not limited to, valdecoxib, rofecoxib, carprofen, celecoxib, lumiracoxib, tolfenamic acid, nimesulide, niflumic acid, asaraldehyde, lornoxicam, sodium meclofenamate, amfenac sodium hydrate, diclofenac sodium, ketoprofen, ketorolac, naproxen sodium, indomethacin, ibuprofen, aspirin, mefenamic acid, bromfenac sodium, oxaprozin, zaltoprofen, and nepafenac.

Examples of the “CXCR4 inhibitor” include, but are not limited to, WZ811, plerixafor (AMD3100), and plerixafor 8HCl (AMD3100 8HCl).

The coadministration drug may also be one or more drugs selected from the group consisting of “hormone therapy agent”, “immunotherapeutic agent”, “biopharmaceutical”, “cell growth factor”, “cell growth factor inhibitor”, “cell growth factor receptor inhibitor”, “radiotherapeutic agent”, “auxiliary agent”, and “chemotherapeutic agent”. For example, one to five drugs, one to three drugs, or one drug selected from the above group of drugs may be used in combination with the peptide or the compound of formula (1), or a pharmaceutically acceptable salt thereof, or a combination thereof as described herein.

Examples of the “hormone therapy agent” include adrenal cortical hormone agents (e.g., steroidal anti-inflammatory agents, estrogen preparations, progesterone preparations, and androgen preparations), anti-estrogen agents, estrogen-controlling agents, estrogen synthesis inhibitors, anti-androgen agents, androgen-controlling agents, androgen synthesis inhibitors, LH-RH agonist preparations, LH-RH antagonist preparations, aromatase inhibitors, steroid-lactonase inhibitors, contraceptive pills, retinoids, and agents that delay metabolism of a retinoid.

Examples of the “hormone therapy agent” include fosfestrol, diethylstilbestrol, fluoxymesterol, chlorotrianisene, methyl testosterone, medroxyprogesterone acetate, megestrol acetate, chlormadinone acetate, cyproterone acetate, danazol, allylestrenol, gestrinone, mepartricin, raloxifene, ormeloxifene, levormeloxifene, tamoxifen citrate, toremifene citrate, iodoxyfene, contraceptive pills, mepitiostane, testololactone, aminoglutethimide, goserelin acetate, buserelin, leuprorelin, leuprolide, droloxifene, epitiostanol, ethinylestradiol sulfonate, estramustine, fadrozole hydrochloride, anastrozole, tetrazole, ketoconazole, letrozole, exemestane, vorozole, formestane, flutamide, bicalutamide, nilutamide, enzalutamide, mifepristone, finasteride, dexamethasone, prednisolone, betamethasone, triamcinolone, abiraterone, liarozole, bexarotene, and DN101.

Examples of the “immunotherapeutic agent” include picibanil, krestin, sizofiran, lentinan, ubenimex, interferon (IFN)-α, interferon (IFN)-β, interferon (IFN)-γ, interleukin, macrophage colony stimulating factor, granulocyte-colony stimulating factor, erythropoietin, lymphotoxin, BCG vaccine, Corynebacterium parvum, levamisole, polysaccharide K, procodazole, anti-CTLA4 antibody, anti-PD-1 antibody, and TLR agonists (e.g., TLR7 agonists, TLR8 agonists, TLR9 agonists).

Examples of the “biopharmaceutical” include, but are not limited to, interleukin-2 (aldesleukin), interferon-a, interferon-β, interferon-γ, erythropoietin (EPO), granulocyte-colony stimulating factor (filgrastim), granulocyte-macrophage-colony stimulating factor (sargramostim), IL13-PE380QR, Bacille Calmette-Guerin, levamisole, octreotide, CPG7909, Provenge, GVAX, Myvax, Favld, lenalidomide, trastuzumab, rituximab, gemtuzumab ozogamicin, alemtuzumab, endostatin, ibritumomab tiuxetan, tositumomab, cetuximab, zanolimumab, ofatumumab, HGS-ETR1, pertuzumab, M200, SGN-30, matuzumab, adecatumumab, denosumab, zalutumumab, MDX-060, nimotuzumab, MORAb-003, Vitaxin, MDX-101, MDX-010, DPC4 antibodies, NF-1 antibodies, NF-2 antibodies, Rb antibodies, p53 antibodies, WT1 antibodies, BRCA1 antibodies, BRCA2 antibodies, ganglioside (GM2), prostate specific antigens (PSA), α-fetoprotein (AFP), carcinoembryonic antigens (CEA), melanoma-associated antigens (MART-1, gap100, MAGE 1,3 tyrosine), papilloma virus E6 and E7 fragments, and DDS formulations thereof.

Regarding the “cell growth factor”, “cell growth factor inhibitor” and “cell growth factor receptor inhibitor”, the cell growth factor may be any agent that promotes cell proliferation. For example, the cell growth factor may be a peptide that has a molecular weight of not more than 20,000 and can bind to a receptor and function at a low concentration.

Examples of the “cell growth factor” include, but are not limited to, epidermal growth factor (EGF), insulin-like growth factor (IGF (e.g., insulin, IGF-1, and IGF-2)), transforming growth factor (TGF (e.g., TGF-α and TGF-β)), nerve growth factor (NGF), brain-derived neurotrophic factor (BDNF), vascular endothelial growth factor (VEGF), colony stimulating factor (CSF (e.g., granulocyte-colony stimulating factor (G-CSF)), granulocyte-macrophage-colony stimulating factor (GM-CSF)), platelet-derived growth factor (PDGF), erythropoietin (EPO), fibroblast growth factor (FGF (e.g., acidic FGF, basic FGF, keratinocyte growth factor (KGK), and FGF-10)), hepatocyte growth factor (HGF), heregulin, and angiopoietin. The term “cell growth factor” is synonymous with the term “growth factor”.

Examples of the “cell growth factor inhibitor” include, but are not limited to, epidermal growth factor inhibitors (EGF inhibitors), insulin-like growth factor inhibitors (IGF inhibitors), nerve growth factor inhibitors (NGF inhibitors), brain-derived neurotrophic factor inhibitors (BDNF inhibitors), vascular endothelial cell growth factor inhibitors (VEGF inhibitors), colony stimulating factor inhibitors (CSF inhibitors), platelet-derived growth factor inhibitors (PDGF inhibitors), erythropoietin inhibitors (EPO inhibitors), fibroblast growth factor inhibitors (FGF inhibitors), hepatocyte growth factor inhibitors (HGF inhibitors), heregulin inhibitors, and angiopoietin inhibitors. The term “cell growth factor inhibitor” is synonymous with the term “growth factor inhibitor”.

Examples of the “cell growth factor receptor inhibitor” include, but are not limited to, epidermal growth factor receptor inhibitors (EGFR inhibitors), insulin-like growth factor receptor inhibitors (IGFR inhibitors), nerve growth factor receptor inhibitors (NGFR inhibitors), brain-derived neurotrophic factor receptor inhibitors (BDNFR inhibitors), vascular endothelial cell growth factor receptor inhibitors (VEGFR inhibitors), colony stimulating factor receptor inhibitors (CSFR inhibitors), platelet-derived growth factor receptor inhibitors (PDGFR inhibitors), erythropoietin receptor inhibitors (EPOR inhibitors), fibroblast growth factor receptor inhibitors (FGFR inhibitors), hepatocyte growth factor receptor inhibitors (HGFR inhibitors), heregulin receptor inhibitors, and angiopoietin receptor inhibitors. The term “cell growth factor receptor inhibitor” is synonymous with the term “growth factor receptor inhibitor”.

Examples of the “radiotherapeutic agent” include, but are not limited to, radioactive materials and radiosensitizers.

The “auxiliary agent” is an agent used together with an anticancer agent for suppressing a side effect or vomiting caused by the anticancer agent. Examples of the “auxiliary agent” include, but are not limited to, aprepitant, ondansetron, lorazepam, dexamethasone, diphenhydramine, ranitidine, cimetidine, ranitidine, famotidine, cimetidine, Procrit, epoetin alfa, filgrastim, oprelvekin, leucovorin, and granulocyte-macrophage-colony stimulating factor (GM-CSF).

Examples of the “chemotherapeutic agent” include, but are not limited to, alkylating agents, platinum formulations, antimetabolites, topoisomerase inhibitors, DNA intercalators, antimitotic agents, antitumor antibiotics, plant-derived anticancer agents, epigenome drugs, immunomodulators, molecular targeted drugs, angiogenesis inhibitors, and other chemotherapeutic agents. Some typical examples of chemotherapeutic agent are listed below.

Examples of the “alkylating agent” include, but are not limited to, nitrogen mustard, nitrogen mustard N-oxide hydrochloride, chlorambucil, cyclophosphamide, ifosfamide, thiotepa, carboquone, improsulfan tosylate, busulfan, nimustine hydrochloride, mitobronitol, melphalan, dacarbazine, procarbazine, ranimustine, estramustine sodium phosphate, triethylenemelamine, carmustine, lomustine, streptozocin, pipobroman, etoglucid, altretamine, ambamustine, dibrospidium hydrochloride, fotemustine, prednimustine, bendamustine, uramustine, semustine, pumitepa, ribomustin, temozolomide, treosulfan, trofosfamide, zinostatin stimalamer, adozelesin, cystemustine, bizelesin, mechlorethamine, uracil mustard, trabectedin, chlormethine, mannosulfan, triaziquone, procarbazine, canfosfamide, nitrosoureas, and DDS formulations thereof.

Examples of the “platinum formulation” include, but are not limited to, cisplatin, carboplatin, miboplatin, nedaplatin, satraplatin, oxaliplatin, triplatin tetranitrate, and DDS formulations thereof.

Examples of the “antimetabolite” include, but are not limited to, antifolates, pyrimidine metabolism inhibitors, purine metabolism inhibitors, ribonucleotide reductase inhibitors, and nucleotide analogs.

Examples of the “antimetabolite” include, but are not limited to, mercaptopurine, 6-mercaptopurine riboside, thioinosine, methotrexate, pemetrexed, eoshitabin, enocitabine, cytarabine, cytarabine ocfosfate, ancitabine hydrochloride, 5-FU agents (e.g., fluorouracil, Carzonal, Bennan, Lunachol, Lunapon, tegafur, tegafur-uracil, tegafur-gimeracil-oteracil potassium (TS-1), UFT, doxifluridine, carmofur, gallocitabine, emitefur, and capecitabine), aminopterin, nelarabine, leucovorin calcium, Tabloid, butocine, folinate calcium, levofolinate calcium, cladribine, emitefur, fludarabine, gemcitabine, hydroxycarbamide, pentostatin, piritrexim, idoxuridine, mitoguazone, tiazofurine, ambamustine, bendamustine, floxuridine, leucovorin, hydroxyurea, thioguanine, asparaginase, bortezomib, raltitrexed, clofarabine, enocitabine, sapacitabine, azacytidine, sulfadiazine, sulfamethoxazole, trimethoprim, Liproxstatin-1, D4476, Xanthohumol, Epacadostat (INCB024360), Vidofludimus, P7C3, GMX1778 (CHS828), NCT-501, SW033291, Ro61-8048, and DDS formulations thereof.

Examples of the “topoisomerase inhibitor” include, but are not limited to, doxorubicin, daunorubicin, epirubicin, idarubicin, anthracenedione, mitoxantrone, mitomycin C, bleomycin, dactinomycin, plicatomycin, irinotecan, camptothecin, rubitecan, belotecan, etoposide, teniposide, topotecan, amsacrine, and DDS formulations thereof.

Examples of the “DNA intercalator” include, but are not limited to, proflavine, doxorubicin (adriamycin), daunorubicin, dactinomycin, thalidomide, and DDS formulations thereof.

Examples of the “antimitotic agent” include, but are not limited to, paclitaxel, paclitaxel derivatives (e.g., DHA paclitaxel, paclitaxel polyglutamate, nab-paclitaxel, micellar paclitaxel, 7α-glucosyloxyacetylpaclitaxel, and BMS-275183), docetaxel, vinorelbine, vincristine, vinblastine, vindesine, vinzolidine, etoposide, teniposide, ixabepilone, larotaxel, ortataxel, tesetaxel, ispinesib, colchicine, vinflunine, and DDS formulations thereof.

Examples of the “antitumor antibiotic” include, but are not limited to, actinomycin D, actinomycin C, mitomycin C, chromomycin A3, mithramycin A, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, amrubicin hydrochloride, neocarzinostatin, zinostatin stimalamer, mithramycin, sarkomycin, carzinophilin, mitotane, zorubicin hydrochloride, mitoxantrone hydrochloride, idarubicin hydrochloride, liposomal doxorubicin, and DDS formulations thereof.

Examples of the “plant-derived anticancer agent” include, but are not limited to, irinotecan, nogitecan, etoposide, etoposide phosphate, eribulin, sobuzoxane, vinblastine sulfate, vincristine sulfate, vindesine sulfate, teniposide, paclitaxel, paclitaxel injection, docetaxel, DJ-927, vinorelbine, topotecan, and DDS formulations thereof.

Examples of the “epigenome drug” include, but are not limited to, DNA methylation inhibitors, histone deacetylase (HDAC) inhibitors, DNA methyl transferase (DNMT) inhibitors, histone deacetylase activators, histone demethylase inhibitors, and methylated nucleotides.

Specific examples of the “epigenome drug” include, but are not limited to, vorinostat, belinostat, mocetinostat (MGCD0103), entinostat (SNDX-275), romidepsin, azacytidine, decitabine, GSK28′/9552 2H1, SGC707, ORY-1001 (RG-6016), PFI-4, SirReal2, GSK2801, CPI-360, GSK503, AMI-1, CPI-169, and DDS formulations thereof.

Examples of the “immunomodulator” include, but are not limited to, thalidomide, lenalidomide, pomalidomide, and DDS formulations thereof.

The “molecular targeted drug” can be a small compound or an antibody. Examples of the “molecular targeted drug” include, but are not limited to, kinase inhibitors, proteasome inhibitors, monoclonal antibodies, mTOR inhibitors, TNF inhibitors, and T-cell inhibitors.

Examples of the “kinase inhibitor” include, but are not limited to, tyrosine kinase inhibitors, serine/threonine kinase inhibitors, Raf kinase inhibitors, cyclin-dependent kinase (CDK) inhibitors, and mitogen-activated protein kinase (MEK) inhibitors.

Specific examples of the “kinase inhibitor” include, but are not limited to, imatinib, gefitinib, erlotinib, afatinib, dasatinib, bosutinib, vandetanib, sunitinib, axitinib, pazopanib, lenvatinib, lapatinib, nintedanib, nilotinib, crizotinib, ceritinib, alectinib, ruxolitinib, tofacitinib, ibrutinib, sorafenib, vemurafenib, dabrafenib, palbociclib, trametinib, regorafenib, cedivanib, lestaurtinib, bandetinib, vatalanib, seliciclib, tivantinib, canertinib, pelitinib, tesevatinib, cediranib, motesanib, midostaurin, foretinib, cabozantinib, selumetinib, neratinib, volasertib, saracatinib, enzastaurin, tandutinib, semaxanib, alvocidib, ICR-62, AEE788, PD0325901, PD153035, TK787, amcasertib (BBI503), E6201, E7050, and DDS formulations thereof.

Examples of the “proteasome inhibitor” include, but are not limited to, bortezomib, carfilzomib, and DDS formulations thereof.

Examples of the “monoclonal antibody” include, but are not limited to, anti-CD22 antibodies, anti-CD20 antibodies, anti-CD25 antibodies, anti-CD30 antibodies, anti-CD33 antibodies, anti-CD5 antibodies, anti-CD52 antibodies, anti-epidermal growth factor receptor antibodies (EGFR antibodies), anti-vascular endothelial cell growth factor antibodies (VEGF antibodies), anti-TNF-α antibodies, anti-IL-1 receptor antibodies, anti-IL-2 receptor antibodies, anti-IL-5 receptor antibodies, anti-IL-6 receptor antibodies, anti-HER2 antibodies, anti-IgE antibodies, anti-IgG antibodies, anti-RS virus antibodies, anti-CCR4 antibodies, anti-cytotoxic T lymphocyte-associated antigen 4 (CTLA-4, CD152) antibodies, anti-PD-1 antibodies, anti-receptor activator of nuclear factor κB ligand (RANKL) antibodies, anti-c-Met antibodies, and anti-CXCR4 antibodies.

Specific examples of the “monoclonal antibody” include, but are not limited to, ibritumomab tiuxetan, rituximab, cetuximab, infliximab, basiliximab, brentuximab vedotin, tocilizumab, trastuzumab, bevacizumab, omalizumab, mepolizumab, gemtuzumab ozogamicin, palivizumab, ranibizumab, certolizumab, ocrelizumab, mogamulizumab, eculizumab, pertuzumab, alemtuzumab, inotuzumab, panitumumab, ofatumumab, golimumab, adalimumab, ramucirumab, nivolumab, anakinra, denosumab, ipilimumab, pembrolizumab, matuzumab, farletuzumab, MORAb-004, MORA-b009, and DDS formulations thereof.

Examples of the “mTOR inhibitor” include, but are not limited to, everolimus (RAD001), rapamycin (sirolimus), AZD8055, temsirolimus (CC-779, NSC683864), KU-0063794, voxtalisib (XL-765, SAR245409), MHY1485, dactolisib (BEZ235), PI-103, torkinib (PP242), ridaforolimus (deforolimus, MK-8669), INK-128 (MLN0128), Torin1, omipalisib (GSK2126458, GSK458), OSI-027, PF-04691502, apitolisib (GDC-0980, RG7422), GSK1059615, gedatolisib (PF-05212384, PKI-587), WYE-132, PP121, WYE-354, AZD2014, Torin2, WYE-687, CH5132799, WAY-600, ETP-46464, GDC-0349, XL388, zotarolimus (ABT-578), tacrolimus (FK506), BGT226 (NVP-BGT226), Palomid 529 (P529), chrysophanic acid, and DDS formulations thereof.

Examples of the “TNF inhibitor” include, but are not limited to, etanercept, lenalidomide (CC-5013), pomalidomide, thalidomide, necrostatin-1, and QNZ (EVP4593).

Examples of the “T-cell inhibitor” include, but are not limited to, abatacept.

Examples of the “angiogenesis inhibitor” include, but are not limited to, CM101, IFN-α, IL-12, platelet factor-4, suramin, semaxanib, thrombospondin, VEGFR antagonists, combinations of an angiostatic steroid and heparin, cartilage-derived angiogenesis inhibitors, matrix metalloproteinase inhibitors, batimastat, marimastat, angiostatin, endostatin, 2-methoxyestradiol, tecogalan, thrombospondin, αVβ3 inhibitors, linomide, ADH-1, E7820, and DDS formulations thereof.

Examples of the “other chemotherapeutic agent” include, but are not limited to, finasteride, sobuzoxane, obatoclax, efaproxiral, tipifarnib, and lonafarnib.

When the compound of formula (1) or a pharmaceutically acceptable salt thereof as described herein is used in combination with at least one cancer antigen peptide or a pharmaceutically acceptable salt thereof and/or coadministration drug, these active agents may be formulated in separate compositions or incorporated in a single composition. In an embodiment, the compound of formula (1) or a pharmaceutically acceptable salt thereof and a cancer antigen peptide are incorporated in a single composition. In another embodiment, the compound of formula (1) or a pharmaceutically acceptable salt thereof and a cancer antigen peptide are formulated in separate compositions. A composition may comprise one or more compounds of formula (1) or pharmaceutically acceptable salts thereof and/or one or more cancer antigen peptides. A composition comprising the compound of formula (1) or a pharmaceutically acceptable salt thereof or a cancer antigen peptide may be provided together with instructions of dosage and administration for use of the composition in combination with the other active agent. A composition comprising the compound of formula (1) or a pharmaceutically acceptable salt thereof and a composition comprising a cancer antigen peptide may be incorporated in a single kit. Such a kit may further comprise instructions of dosage and administration for use of the compositions in combination, or may be packaged. In administration of more than one active agent in combination, the agents may be administered on the same administration schedule or different administration schedules.

The composition of the disclosure may comprise the compound of formula (1), the peptide, or a pharmaceutically acceptable salt thereof, or a combination thereof as described herein as an active agent together with a pharmaceutically acceptable carrier. Also, the composition of the disclosure may further comprise, or be administered in combination with, an appropriate adjuvant for enhancing the induction of WT1-specific CTLs and/or helper T cells by the composition.

The “pharmaceutically acceptable carrier” refers to a carrier that is non-toxic to a cell or a mammal exposed to the carrier at an amount or concentration it is used. In some embodiments, a pH buffered aqueous solution is used as a pharmaceutically acceptable carrier. Examples of the “pharmaceutically acceptable carrier” include buffering agents (such as phosphate, citrate, lactate, tartrate, trifluoroacetate and other organic acids); antioxidants (such as ascorbic acid); low molecular weight polypeptides (less than about 10 residues); proteins (such as serum albumin, gelatin or immunoglobulin); hydrophilic polymers (such as polyvinylpyrrolidone); amino acids (such as glycine, glutamine, asparagine, arginine, methionine or lysine); monosaccharides, disaccharides and other carbohydrates (such as glucose, mannose or dextrin); chelating agents (such as EDTA); sugar alcohols (such as mannitol, trehalose or sorbitol); stabilizers (such as diethylenetriaminepentaacetic acid); salt forming counterions (such as sodium); solubilizing agents (such as polysorbate 80®), and/or nonionic surfactants (such as TWEEN®, polyethylene glycol (PEG) and PLURONICS®). A macromolecular material that is metabolized slowly, such as a protein, a polypeptide, a liposome, a polysaccharide, polylactide, polyglycolic acid, polymeric amino acids, amino acid copolymers, and inactive virus particles may also be useful as a pharmaceutically acceptable carrier. For administration, the compound of formula (1) or the peptide as described herein may be formulated in a liposome preparation, attached to beads having a diameter of a micrometer order, or associated with a lipid carrier.

The adjuvant may be any of adjuvants as described in Clin. Microbiol. Rev., 7: 277-289, 1994. Specifically, the adjuvant may be a microorganism-derived agent, GM-CSF, a cytokine such as interleukin-2, interleukin-7, or interleukin-12, a plant-derived agent, a marine organism-derived agent, a mineral gel such as aluminum hydroxide, lysolecithin, a surfactant such as pluronic polyol, a polyanion, a peptide, or an oil emulsion (an emulsion preparation). Examples of the microorganism-derived agent include lipid A, monophosphoryl lipid A, which is a derivative of lipid A, killed bacteria (e.g., Mycobacterium bacteria such as BCG bactera), bacterium-derived proteins, polynucleotides, Freund's incomplete adjuvant, Freund's complete adjuvant, cell wall skeleton components (e.g., BCG-CWS), trehalose dimycolate (TDM).

The adjuvant may also be a sedimentary adjuvant or an oil adjuvant. A sedimentary adjuvant can be a suspension of an inorganic substance to which a peptide can be adsorbed. Examples of the sedimentary adjuvant include sodium hydroxide, aluminum hydroxide (Alum), calcium phosphate, aluminum phosphate, alum, Pepesu, and carboxyvinyl polymer. An oil adjuvant can be an oil emulsifier that is able to emulsify a peptide by forming micelles comprising an aqueous peptide solution phase encapsulated in a mineral oil membrane. Examples of the oil adjuvant include, but are not limited to, liquid paraffin, lanolin, Freund's adjuvant (Freund's complete adjuvant, and Freund's incomplete adjuvant), Montanide, and a W/O emulsion (see WO2006/078059).

The composition of the disclosure may be provided as a dosage form for oral administration or parenteral administration. Examples of dosage forms for parenteral administration include an injectable preparation, an external preparation, a suppository, an inhalable preparation, or a nasal preparation. In a preferred embodiment, the composition of the disclosure is provided as an injectable preparation.

An injectable preparation may be in the form of a solution, a suspension, or an emulsion, which comprises one or more active agents dissolved, dispersed or emulsified in a liquid for injection, or may be provided as a solid formulation comprising active agent(s) to be dissolved or dispersed in a liquid for injection before use. A liquid for injection may comprise distilled water for injection, physiological saline, a vegetable oil, propylene glycol, polyethylene glycol, an alcohol such as ethanol, or a combination thereof. An injectable preparation may additionally comprise a stabilizer, a solubilizing aid (such as glutamic acid, aspartic acid, or polysorbate 80®), a dispersant, an emulsifier, an analgesic, a buffer, a preservative, or other appropriate additive. For providing an injectable preparation as a sterilized preparation, it may be subjected to sterilization in the final step of its production, or produced aseptically throughout its production. A formulation for injection may be provided as a sterilized solid formulation, for example a lyophilized formulation, which may be reconstituted in sterilized water for injection or other appropriate sterilized liquid before use.

An external preparation may be in the form of an ointment, a gel, a cream, a plaster, a patch, a liniment, a spray, an inhalant, an aerosol, an eye drop, or a nasal drop, which may be prepared in accordance with a conventionally known preparation method, and may comprise one or more active agents.

An ointment may be prepared in accordance with a conventionally known preparation method, for example by incorporating one or more active agents in an ointment base by grinding or melting. For preparing an ointment, any conventionally used ointment base may be used, which may comprise a higher fatty acid or fatty acid ester (such as adipic acid, myristic acid, palmitic acid, stearic acid, or oleic acid, or an ester thereof), a wax (such as beeswax, spermaceti, or ceresin), a surfactant (such as polyoxyethylene alkyl ether phosphate), a higher alcohol (such as cetanol, stearyl alcohol, or cetostearyl alcohol), a silicone oil (such as dimethylpolysiloxane), a hydrocarbon (such as a hydrophilic petrolatum, white petrolatum, purified lanolin, or liquid paraffin), a glycol (such as ethylene glycol, diethylene glycol, propylene glycol, polyethylene glycol, or macrogol), a vegetable oil (such as castor oil, olive oil, sesame oil, or turpentine oil), an animal oil (such as mink oil, egg-yolk oil, squalane, or squalene), water, an absorption enhancer, a skin protective agent, or a combination thereof. An ointment may additionally comprise a humectant, a preservative, a stabilizer, an antioxidant, a fragrance, or other appropriate additive.

The pharmaceutical composition in a gel form may be prepared in accordance with a conventionally known preparation method, for example by incorporating one or more active agents in a gel base by melting. For preparing a gel, any conventionally used pharmaceutical gel base may be used, which may comprise a lower alcohol (such as ethanol, or isopropyl alcohol), a gelling agent (such as carboxymethyl cellulose, hydroxyethyl cellulose, hydroxypropyl cellulose, or ethyl cellulose), a neutralizing agent (such as triethanolamine, or diisopropanolamine), a surfactant (such as polyoxyethylene glycol monostearate), a gum, water, an absorption enhancer, a skin protective agent, or a combination thereof. A gel may additionally comprise a preservative, an antioxidant, a fragrance, or any other appropriate additive(s).

The pharmaceutical composition in a cream form may be prepared in accordance with a conventionally known preparation method, for example by incorporating one or more active agents in a pharmaceutical cream base by melting or emulsification. For preparing a cream, any conventionally used pharmaceutical cream base may be used, which may comprise a higher fatty acid ester, a lower alcohol, a hydrocarbon, a polyhydric alcohol (such as propylene glycol, or 1,3-butylene glycol), a higher alcohol (such as 2-hexyldecanol, or cetanol), an emulsifier (such as a polyoxyethylene alkyl ether, or a fatty acid ester), water, an absorption enhancer, a skin protective agent, or a combination thereof. A cream may additionally comprise a preservative, an antioxidant, a fragrance, or other appropriate additive.

The pharmaceutical composition in the form of a plaster may be prepared in accordance with a conventionally known preparation method, for example by incorporating one or more active agents in a plaster base by melting, and applying the mixture onto a support. For preparing a plaster, any conventionally used pharmaceutical plaster base may be used, which may comprise a thickening agent (such as polyacrylic acid, polyvinylpyrrolidone, gum arabic, starch, gelatin, or methyl cellulose), a humectant (such as urea, glycerol, or propylene glycol), a filler (such as kaolin, zinc oxide, talc, calcium, or magnesium), water, a solubilizing aid, a tackifier, a skin protective agent, or a combination thereof. A plaster may additionally comprise a preservative, an antioxidant, a fragrance, or other appropriate additive.

The pharmaceutical composition in the form of a patch may be prepared in accordance with a conventionally known preparation method, for example by incorporating one or more active agents in a patch base by melting, and applying the mixture onto a support. For preparing a patch, any conventionally used pharmaceutical patch base may be used, which may comprise a polymer, an oil or fat, a higher fatty acid, a tackifier, a skin protective agent, or a combination thereof. A patch may additionally comprise a preservative, an antioxidant, a fragrance, or other appropriate additive.

The pharmaceutical composition in the form of a liniment may be prepared in accordance with a conventionally known preparation method, for example by dissolving, dispersing or emulsifying one or more active agents in a vehicle that may comprise water, an alcohol (such as ethanol, or polyethylene glycol), a higher fatty acid, glycerol, soap, an emulsifier, a dispersant, or a combination thereof. A liniment may additionally comprise a preservative, an antioxidant, a fragrance, or other appropriate additive.

The pharmaceutical composition in the form of a spray, or an inhalant may comprise active agent(s), and optionally a stabilizing agent such as sodium hydrogen sulfite, or a tonicity agent or buffer, such as sodium chloride, sodium citrate or citric acid, in a vehicle.

The pharmaceutical composition in a dosage form for inhalation may be in the form of an aerosol, an inhalable powder, or an inhalable liquid, or may be provided as a liquid concentrate that is to be dissolved or dispersed in water or other appropriate vehicle to form an inhalable preparation before use. A preparation for inhalation may be prepared in accordance with a conventionally known preparation method. An inhalable liquid may optionally comprise a preservative (such as benzalkonium chloride, or paraben), a coloring agent, a buffer (such as sodium phosphate, or sodium acetate), a tonicity agent (such as sodium chloride, or concentrated glycerin), a thickening agent (such as a carboxyvinyl polymer), an absorption enhancer, or other appropriate additive. An inhalable powder may optionally comprise a lubricant (such as stearic acid, or a salt thereof), a binder (such as starch, or dextrin), a filler (such as lactose, or cellulose), a coloring agent, a preservative (such as benzalkonium chloride, or paraben), an absorption enhancer, or other appropriate additive. For administration of an inhalable liquid, a spray device (such as an atomizer, or a nebulizer) is usually used. An inhalable powder is usually dispensed from a powder inhalation device.

The pharmaceutical composition in the form of a spray may comprise active agent(s), and optionally a stabilizing agent (such as sodium hydrogen sulfite), or a tonicity agent or buffer (such as sodium chloride, sodium citrate, or citric acid) in a vehicle. A spray may be prepared in accordance with a preparation method as described, for example, in U.S. Pat. No. 2,868,691, or U.S. Pat. No. 3,095,355.

The pharmaceutical composition may be prepared in other parenteral dosage form. For example, one or more active agents may be formulated into a rectal suppository or a vaginal pessary by a conventionally known preparation method.

In one embodiment, the composition comprising the compound of formula (1) or the peptide, or a pharmaceutically acceptable salt thereof, or a combination thereof as described herein comprises one or more pharmaceutically acceptable carriers selected from the group consisting of trehalose, mannitol, methionine, citric acid, lactic acid, tartaric acid, acetic acid, trifluoroacetic acid, and a pH adjusting agent.

The compound of formula (1) or the peptide, or a pharmaceutically acceptable salt thereof, or a coadministration drug as described herein can be administered to a subject by an appropriate method depending on the disease to treat, condition of the subject, target site of the administration, or other factors. For example, parenteral administration, preferably, intravenous, intramuscular, intradermal, or subcutaneous administration by injection or infusion may be useful. The compound or peptide as described herein may be administered in a lymphocyte therapy or a DC (dendritic cell) therapy. When an immunomodulator is coadministered, the immunomodulator may be administered transdermally, or transmucosally by intranasal, buccal, vaginal, rectal, or sublingual administration.

Frequency of dose, or dosing interval may be appropriately selected depending on the disease to treat, condition of the subject, route of administration, or other factor. Administration is usually repeated, preferably every few days or few months.

The compound of formula (1) or the peptide or a pharmaceutically acceptable salt thereof, or a coadministration drug, if any, as described herein can be administered to a subject in an appropriate amount depending on the disease to treat, condition of the subject, route of the administration, or other factor. The compound or the peptide or a pharmaceutically acceptable salt thereof may usually be administered in an amount of 0.0001 mg to 1000 mg, preferably 0.001 mg to 1000 mg, more preferably 0.1 mg to 10 mg, at one time. A coadministration drug may be administered in an amount appropriately selected on the basis of a known clinical dose of the drug. For example, an immunomodulator as a coadministration drug may usually be administered in an amount of 0.0001 mg to 1000 mg per kg body weight, preferably 0.001 mg to 1000 mg per kg body weight, more preferably 0.1 mg to 10 mg per kg body weight.

When more than one active agent is incorporated in a single composition, they may be incorporated at an amount ratio appropriately selected depending on the disease to treat, condition of the subject, route of administration, or other factor. For example, for treating a human subject by administration of a composition comprising the peptide and/or the compound as described herein and an immunomodulator or other coadministration drug, the immunomodulator or coadministration drug may be used in an amount of 0.01 to 100 parts by weight relative to the peptide and/or the compound.

The term “subject” as used herein includes human and non-human mammals. Non-human mammals include, but are not limited to, non-human primate, ovine, canine, feline, equine, and bovine. A human subject, especially a human subject in need of potentiation of immune response is preferred.

The term “effective amount” as used herein means an amount of an active agent that completely or partially inhibits the progression of a cancer, or at least partially reduces one or more symptoms of a cancer. An effective amount may be a therapeutically or prophylactically effective amount. An effective amount of an agent is determined depending on the age or sex of the subject, the type or severity of condition to treat with the agent, a desired outcome of the treatment with the agent, or other factor. A person skilled in the art can determine an effective amount for a particular patient.

The compound of formula (1) or the peptide, or a pharmaceutically acceptable salt thereof, or a combination thereof as described herein may be administered in combination with a non-drug therapy, or even more than one non-drug therapy selected, for example, from surgery, radiotherapy, gene therapy, hyperthermia, cryotherapy, or laser burning therapy. For example, the compound of formula (1) or the peptide, or a pharmaceutically acceptable salt thereof, or a combination thereof as described herein may be administered before or after a non-drug therapy such as surgery, or before or after a combination of two or three non-drug therapies.

The compound of formula (1) or the peptide, or a pharmaceutically acceptable salt thereof, or a combination thereof as described herein can be further used in combination with an agent to reduce an unwanted side effect, if any, such as an antiemetic agent, sleep-inducing agent, or anticonvulsant.

The present disclosure also provides a polynucleotide encoding a peptide as described herein. The polynucleotide may be DNA or RNA. The polynucleotide has a nucleotide sequence corresponding to the amino acid sequence of the peptide the polynucleotide encodes. The polynucleotide may be synthesized by a method for DNA or RNA synthesis, or PCR.

The polynucleotide of the disclosure includes a polynucleotide which is able to hybridize to a complementary sequence of a polynucleotide encoding the peptide under a stringent condition, and encodes a peptide having a similar ability of inducing CTLs or helper T cells to the peptide. The hybridization under a stringent condition may be conventional hybridization as described in Sambrook J., Frisch E. F., Maniatis T., Molecular Cloning 2nd edition, Cold Spring Harbor Laboratory press. A “stringent condition” may be created, for example in a solution of 6×SSC (in this regard, 10×SSC contains 1.5 M NaCl, and 0.15 M trisodiun citrate) with 50% formamide at 45° C. for forming a hybrid, and in a solution of 2×SSC for washing a hybrid (Molecular Biology, John Wiley & Sons, N.Y. (1989), 6.3.1-6.3.6).

The present disclosure also provides an expression vector comprising the polynucleotide of the disclosure (hereinafter also referred to as a WT1 expression vector). The expression vector may be of any appropriate type, and have any appropriate sequence outside the sequence of the polynucleotide of the disclosure, depending on type of a host to be transfected with the vector or any other specific factors. The vector may be a plasmid, a phage vector, or a viral vector. For transfection to E. coli, a plasmid vector such as pUC128, pUC119, pBR322 or pCR3, or a phage vector such as λZAPII or λgt11 may be used. For transfection to yeast, pYES2 or pYEUra3 may be used. For transfection to insect cells, pAcSGHisNT-A may be used. For transfection to animal cells, a plasmid vector such as pKCR, pCDM8, pGL2, pcDNA3.1, pRc/RSV or pRc/CMV, or a viral vector such as a retroviral vector, an adenoviral vector, or an adeno-associated viral vector may be used. The vector may further comprise a promotor for expression induction, a gene coding a signal sequence, a marker gene for selection, or a terminator. The vector may also comprise a sequence encoding a tag such as thioredoxin, a His tag or GST (glutathione S-transferase) so that a protein is expressed with a tag fused thereon for facilitation of isolation or purification of the protein. Such a vector may comprise an appropriate promotor (such as lac, tac, trc, trp, CMV, SV40 early promoter) depending on a host to be transfected with the vector, and can be a GST-fused protein expression vector (for example pGEX4T), a vector comprising a sequence of a tag such as Myc or His (for example, pcDNA3.1/Myc-His), or an expression vector for a protein fused to thioredoxin or a His tag (for example, pET32a).

The expression vector of the disclosure expresses the peptide as described herein in vitro or in vivo, and therefore, is useful for treatment or prevention of a cancer.

The present disclosure further provides an antibody against the peptide as described herein. The antibody may be polyclonal or monoclonal, and can be prepared in accordance with a conventional method for preparation of a polyclonal or monoclonal antibody (Current protocols in Molecular Biology edit. Ausubel et al. (1987) Publish. John Wiley and Sons. Section 11.12-11.13, Antibodies; A Laboratory Manual, Lane, H, D. et al (Ed.), Cold Spring Harber Laboratory Press, New York 1989). The antibody can be obtained as an antibody which recognizes the peptide of the disclosure, or an antibody which recognizes and neutralizes the peptide of the disclosure, from an animal immunized by a conventional method with the compound or peptide as described herein. The antibody can be used in affinity chromatography, or in immunological diagnosis based, for example, on immunoblotting, radioimmunoassay (RIA), enzyme-linked immunosorbent assay (ELISA), or fluorescent or luminescent immunoassay, for a cancer, especially a cancer associated with WT1 gene expression or an elevated level of WT1 gene expression.

The compound of formula (1) of the disclosure efficiently induces CTLs, has favorable physicochemical stability, enables easy manufacturing and simple manufacturing control, and can be used widely. Also, the compound of formula (1) of the disclosure induces CTLs more efficiently when combined with a different cancer antigen peptide.

### EXAMPLES

The present invention is described in further detail in the following Examples, which are not in any way intended to limit the scope of the invention.

### Reference Example 1

### Synthesis of a Peptide Consisting of the Amino Acid Sequence: RMFPNAPYL (Arg-Met-Phe-Pro-Asn-Ala-Pro-Tyr-Leu) (SEQ ID NO: 2)

The peptide was synthesized by an Fmoc/tBu method for solid-phase peptide synthesis. Specifically, peptide chain elongation was performed by using as a starting material 1.00 g of Fmoc-Leu-Alko-PEG resin (wherein Fmoc represents 9-fluorenylmethyloxycarbonyl, Alko represents p-alkoxybenzyl alcohol, and PEG represents polyethylene glycol) (Watanabe Chemical Industries; 0.23 mmol/g, 0.23 mmol) in CS Bio CS336X peptide synthesizer. For removing the Fmoc protecting group, the resin was treated with a solution of 20% piperidine in N,N-dimethylformamide (DMF) for 5 minutes and 20 minutes. For coupling a protected amino acid to the resin, a solution of 1.05 mmol of a protected amino acid, 1 mol of O-(benzotriazol-1-yl)-N,N,N′,N′-tetramethyluronium hexafluorophosphate (HBTU), 2 mmol of N,N-diisopropylethylamine (DIPEA) in DMF was added to the resin and reacted for one hour. The resin from the reaction was washed with DMF and diethyl ether, and dried under vacuum. The resin to which a synthesized peptide chain was attached was incubated under shaking for two hours in 10 ml of a mixture of trifluoroacetic acid (TFA)/water/triisopropylsilane (TIS) (volume ratio: 94/2.5/2.5) at room temperature. The resin was then filtered off. The filtrate was concentrated under vacuum, and then cooled on ice and diluted in 50 ml of diethyl ether to precipitate the peptide. The precipitated peptide was filtered, washed with ether, and dried under vacuum. A crude peptide product thus obtained was dissolved in a mixture of 20% acetic acid/acetonitrile (volume ratio 1/1) and subjected to purification under the conditions described below. The peptide consisting of the sequence: RMFPNAPYL (Arg-Met-Phe-Pro-Asn-Ala-Pro-Tyr-Leu) (SEQ ID NO: 2) was obtained as a TFA salt (0.16 g).

Purification Conditions:

HPLC System: Gilson high throughput preparative HPLC system Column: YMC ODS-A 3 cmϕ×25 cm, 10 μm
Eluent 1: 0.1% TFA in water
Eluent 2: 0.035% TFA in acetonitrile
Flow rate: 20 ml/min

### Gradient Method:

TABLE 45

| Time (min) | Concentration of Eluent 2 (%) |
|---|---|
| 0 | 10 |
| 25 | 50 |

Identification of the product was conducted by analysis under the following conditions:

MS System: Shimazu LCMS-IT-TOF system
Column: Kinetex Minibore column, 2.1 mmϕ×50 mm, 1.7 μm
Eluent 1: 0.1% formic acid in water
Eluent 2: 0.1% formic acid in acetonitrile
Flow rate: 1.2 ml/min

### Gradient Method:

TABLE 46

| Time (min) | Concentration of Eluent 2 (%) |
|---|---|
| 0 | 10 |
| 1.4 | 95 |
| 1.6 | 95 |

MS: m/z=554.73 [M+2H]²⁺, retention time: 0.82 min.

### Reference Example 2

In accordance with the procedure as described in Reference Example 1, the peptide as shown in Table 47 was synthesized from corresponding starting materials and obtained as a TFA salt.

TABLE 47

| Reference | | | LC-TOFMS (m/z, |
|---|---|---|---|
| example | SEQ ID | Amino acid | retention time |
| No. | NO: | sequence | (min)) |
| 2 | 4 | CYTWNQMNL | 586.69 [M + 2H]²⁺, |
| | | | 0.87 |

### Example 1

In accordance with the procedure as described in WO2014/157692, the compound as shown in Table 48 was obtained as a TFA salt (in the structural formula shown in Table 48, C—C means that the C residues are linked together by a disulfide bond.).

TABLE 48

| | | | LC-TOFMS |
|---|---|---|---|
| Example | Formula | | (m/z, retention |
| No. | NO. | Structural formula | time (min)) |
| 1 | 5 | 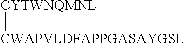 | 1031.76 [M + 3H]³⁺, 1.09 |

### Experimental Example 1

Evaluation of CTL Inducing Ability In Vivo in HLA-A2402 Transgenic Mouse

The peptide of SEQ ID NO: 4 synthesized in Reference Example 2 and the compound of formula (5) synthesized in Example 1 were evaluated for ability to induce CTLs in vivo in an HLA-A2402 transgenic mouse. The peptide of SEQ ID NO: 4 and the sequence CYTWNQMNL (SEQ ID NO: 4) included in the compound of formula (5) corresponds to an HLA-A2402-restricted WT1 peptide.

The HLA-A2402 transgenic mouse (C57BL/6CrHLA-A2402/K^{b}) is a mouse that expresses a chimeric HLA of a human MHC, HLA-A2402 with a mouse MHC, H-2K^{b}. The mouse is useful for screening peptides for potential ability to induce CTLs in HLA-A2402-positive humans (Int J Cancer. 2002; 100:565-70).

Induction of CTLs specific to the peptide of SEQ ID NO: 4 by the peptide of SEQ ID NO: 4 or the compound of formula (5) in the mouse was determined by measuring the level of IFNγ produced by splenocytes from the mouse to which the peptide of SEQ ID NO: 4 or the compound of formula (5) had been administered upon stimulation of the cells with the peptide of SEQ ID NO: 4.

The peptide of SEQ ID NO: 4 was dissolved in DMSO at a concentration of 66.67 mg/mL. The solution was diluted with water for injection to a concentration of 5.0 mg/mL, and then converted to an emulsion by addition of an equal volume of Montanide ISA 51 VG. The emulsion was injected to mice intradermally at two sites in the tail base area in an amount for administering 250 μg of the peptide per site. On the other hand, the compound of formula (5) was dissolved in DMSO at a concentration of 351.1 mg/mL, and the solution was diluted with water for injection to a concentration of the compound of formula (5) of 13.2 mg/mL, and then converted into an emulsion by the addition of an equal volume of Montanide ISA 51 VG. The emulsion was injected to mice intradermally at two sites in the tail base area in an amount for administering 660 μg of the compound per site. The molar ratio of the peptide of SEQ ID NO: 4 and the peptide contained in the compound of formula (5) administered per mouse was almost 1:1. One week after the administration, the mice were sacrificed with CO₂ gas. Splenocytes were harvested from spleens removed from the mice. The splenocytes were then frozen-stored at −80° C. overnight. For measuring IFNγ-production, an IFNγ ELISPOT assay kit was used. In particular, an ELISPOT plate was treated with an anti-mouse-IFNγ antibody on the day before preparation of the splenocyte samples. On the next day, the plate was blocked by treatment with an RPMI 1640 medium with 10% FBS. The frozen splenocytes from the HLA-A2402 transgenic mice were initiated and added to the blocked ELISPOT plate at 2.5×10⁵ cells/well. For in vitro stimulation of the cells, the peptide of SEQ ID NO: 4 was dissolved in DMSO at a concentration of 40 mg/ml, diluted with RPMI 1640 with 10% FBS to 40 μg/ml, and added to the splenocyte-containing wells at a final concentration of 10 μg/ml. The plate was incubated for about 17 hours at 37° C. under an atmosphere of 5% CO₂. Then, after removal of the culture medium from the wells, the ELISPOT plate was subjected to treatment for cell staining in accordance with the manufacturer's protocol. Stained spots were counted on ImmunoSpot Analyzer (C.T.L.).

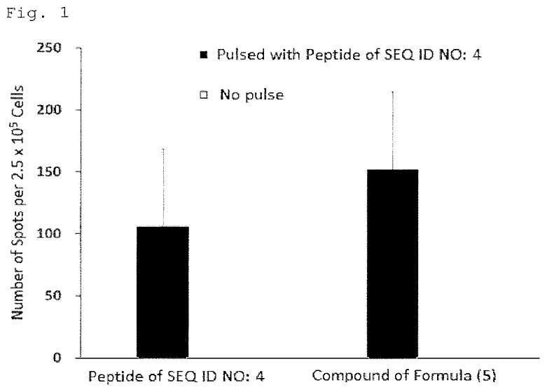

FIG. 1 shows results from the IFNγ ELISPOT assay using the HLA-A2402 transgenic mouse. The scale on the vertical axis of the graph of FIG. 1 indicates the number of cells (CTLs) that produced IFNγ in response to the stimulation with the peptide of SEQ ID NO: 4, which were comprised in the cells seeded on the plate. The peptide administered to the mice are described under the bars. The black bar of FIG. 1 shows the number of splenocytes of the HLA-A2402 transgenic mouse that produced IFNγ in response to the stimulation with the peptide of SEQ ID NO: 4. The white bar shows the number of splenocytes of the HLA-A2402 transgenic mouse that produced IFNγ in the absence of the peptide stimulation. Therefore, the difference in cell count between the black bar and the white bar shows the number of CTLs specific to the peptide. The results show that the peptide of SEQ ID NO: 4 or the compound of formula (5) induced CTLs responsive to the peptide of SEQ ID NO: 4 in the HLA-A2402 transgenic mouse, and that many CTLs responsive to the peptide of SEQ ID NO: 4 were found when the compound of formula (5) was administered as compared with the peptide of SEQ ID NO: 4.

The results demonstrate that the compound of formula (5) can induce CTLs specific to the peptide of SEQ ID NO: 4. The results also demonstrate that the compound of formula (5) is a potent inducer for CTLs responsive to the peptide of SEQ ID NO: 4.

### Experimental Example 2

Evaluation of CTL Inducing Ability In Vivo in HLA-A0201 Transgenic Mouse

In accordance with the procedure as described in Experimental Example 1, except for the conditions as described below, a cocktail vaccine of the compound of the formula (5) synthesized in Example 1 and the peptide of SEQ ID NO: 2 synthesized in Reference Example 1 was evaluated for ability to induce CTLs in vivo in an HLA-A0201 transgenic mouse. The sequence CYTWNQMNL (SEQ ID NO: 4) included in the compound of formula (5) corresponds to an HLA-A2402-restricted WT1 peptide and the sequence RMFPNAPYL (SEQ ID NO: 2) corresponds to an HLA-A0201-restricted WT1 peptide.

The HLA-A0201 transgenic mouse (C57BL/6CrHLA-A2.1DR1) lacks mouse MHC, and instead expresses a chimeric HLA of a human MHC, HLA-A0201, with a mouse MHC, H-2D^{b}, and HLA-DRB1*0101. The mouse is useful for screening peptides for potential ability to induce CTLs in HLA-A0201-positive humans (Eur J Immunol. 2004; 34: 3060-9), and also for evaluating helper peptides that can bind to the human MHC HLA-DRB1*0101 and induce helper T cells for ability to enhance CTL induction.

Induction of CTLs specific to the peptide of SEQ ID NO: 2 by the cocktail vaccine of the compound of formula (5) and the peptide of SEQ ID NO: 2 in the mouse was measured, compared with a vaccine comprising the peptide of SEQ ID NO: 2 alone, as the level of IFNγ produced by splenocytes from the mouse upon stimulation of the cells with the peptide of SEQ ID NO: 2. Increase in the number of CTLs specific to the peptide of SEQ ID NO: 2 in a sample from a mouse treated with the cocktail vaccine comprising the compound of formula (5) and the peptide of SEQ ID NO: 2 compared with a sample from a mouse treated with the vaccine comprising the peptide of SEQ ID NO: 2 alone reflects the effect of co-administration of the compound of formula (5) and the peptide of SEQ ID NO: 2 on the CTL induction.

The peptide of SEQ ID NO: 2 was dissolved in dimethyl sulfoxide (DMSO) at a concentration of 4.0 mg/ml. The solution was diluted with water for injection to a concentration of 3.0 mg/ml, and then converted to an emulsion by addition of an equal volume of Montanide ISA 51 VG. The emulsion was injected to mice intradermally at two sites in the tail base area in an amount for administering 150 μg of the peptide per site. On the other hand, a solution of the compound of formula (5) (222.2 mg/ml) and the peptide of SEQ ID NO: 2 (80 mg/ml) in DMSO was prepared, diluted with water for injection to concentrations of the compound of formula (5) of 8.4 mg/ml and the peptide of SEQ ID NO: 2 of 3.0 mg/ml, and then converted into an emulsion by the addition of an equal volume of Montanide ISA 51 VG. The cocktail vaccine emulsion was injected to mice intradermally at two sites in the tail base area in an amount for administering 420 μg of the compound of formula (5) per site and 150 μg of the peptide of SEQ ID NO: 2 per site. The molar ratio of each peptide contained in the compound of formula (5) and the peptide of SEQ ID NO: 2 administered per mouse was almost 1:1. The frozen splenocytes from the HLA-A0201 transgenic mice were initiated and added to the blocked ELISPOT plate at 2.5×10⁵ cells/well.

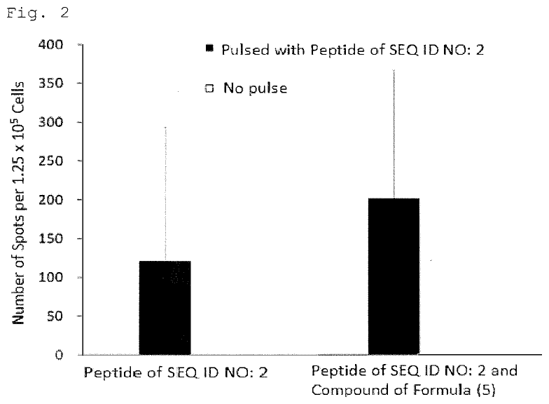

FIG. 2 shows results from the IFNγ ELISPOT assay using the HLA-A0201 transgenic mouse. The results show that the peptide of SEQ ID NO: 2 or the cocktail vaccine of the compound of formula (5) and the peptide of SEQ ID NO: 2 induced CTLs responsive to the peptide of SEQ ID NO: 2 in the HLA-A0201 transgenic mouse, and that the CTL induction by the peptide of SEQ ID NO: 2 was enhanced by the cocktail vaccine of the compound of formula (5) and the peptide of SEQ ID NO: 2.

The results suggest that the compound of formula (5) has an ability to enhance peptide-specific CTL induction by the peptide of SEQ ID NO: 2, in other words, a helper function.

### Reference Examples 3 and 4

In accordance with the procedure as described in WO2014/157692, the compounds as shown in Table 49 were obtained as TFA salts (in the structural formula shown in Table 49, C—C means that the C residues are linked together by a disulfide bond.). These compounds are not encompassed in the compound of the disclosure and thus indicated as reference examples.

TABLE 49

| Reference | | | LC-TOFMS |
|---|---|---|---|
| example | Formula | | (m/z, retention |
| No. | NO. | Structural formula | time (min)) |
| 3 | 8 | 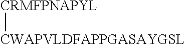 | 1044.74[M + 3H]³⁺_{,} 1.05 |
| 4 | 9 | 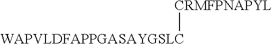 | 1044.73[M + 3H]³, 1.05 |

### Experimental Example 3

Evaluation of In Vivo CTL Induction in HLA-A2402 Transgenic Mouse

A cocktail vaccine of the peptide of SEQ ID NO: 4 synthesized in Reference Example 2 and the compound of formula (8) synthesized in Reference Example 3 is evaluated for ability to induce CTLs in vivo in an HLA-A*24:02 transgenic mouse. The sequence CYTWNQMNL (SEQ ID NO: 4) corresponds to an HLA-A*24:02-restricted WT1 peptide and the sequence RMFPNAPYL (SEQ ID NO: 2) included in the compound of formula (8) corresponds to an HLA-A*02:01-restricted WT1 peptide.

The HLA-A*24:02 transgenic mouse used in Experimental Example 1 is used. Induction of CTLs specific to the peptide of SEQ ID NO: 4 by the peptide of SEQ ID NO: 4 or the cocktail vaccine of the compound of formula (8) and the peptide of SEQ ID NO: 4 in the mouse is determined by measuring the level of IFNγ produced by splenocytes from the mouse to which the peptide of SEQ ID NO: 4 or the cocktail vaccine of the compound of formula (8) and the peptide of SEQ ID NO: 4 has been administered upon stimulation of the cells with the peptide of SEQ ID NO: 4. Increase in the number of CTLs specific to the peptide of SEQ ID NO: 4 in a sample from a mouse treated with the cocktail vaccine comprising the compound of formula (8) and the peptide of SEQ ID NO: 4 compared with a sample from a mouse treated with the vaccine comprising the peptide of SEQ ID NO: 4 alone reflects the effect of co-administration of the compound of formula (8) and the peptide of SEQ ID NO: 4 on the CTL induction.

The peptide of SEQ ID NO: 4 is dissolved in DMSO at a concentration of 66.67 mg/mL. The solution is diluted with water for injection to a concentration of 5.0 mg/mL, and then converted to an emulsion by addition of an equal volume of Montanide ISA 51 VG. On the other hand, a solution of the compound of formula (8) (355.56 mg/ml) and the peptide of SEQ ID NO: 4 (133.33 mg/ml) in DMSO is prepared, diluted with water for injection to concentrations of the compound of formula (8) of 13.4 mg/ml and the peptide of SEQ ID NO: 4 of 5.0 mg/ml, and then converted into an emulsion by the addition of an equal volume of Montanide ISA 51 VG.

The emulsion of the peptide of SEQ ID NO: 4 is injected to mice intradermally at two sites in the tail base area in an amount for administering 250 μg of the peptide per site. The cocktail vaccine emulsion is injected to mice intradermally at two sites in the tail base area in an amount for administering 670 μg of the compound of formula (8) per site and 250 μg of the peptide of SEQ ID NO: 4 per site. The molar ratio of each peptide included in the compound of formula (8) and the peptide of SEQ ID NO: 4 administered per mouse is almost 1:1. One week after the administration, the mice are sacrificed with CO₂ gas. Splenocytes are harvested from spleens removed from the mice. The splenocytes are then frozen-stored at −80° C. overnight. For measuring IFNγ-production, an IFNγ ELISPOT assay kit is used. In particular, an ELISPOT plate is treated with an anti-mouse-IFNγ antibody on the day before preparation of the splenocyte samples. On the next day, the plate is blocked by treatment with an RPMI 1640 medium with 10% FBS. The frozen splenocytes from the HLA-A*24:02 transgenic mice are initiated and added to the blocked ELISPOT plate at 2.5×10⁵ cells/well. For in vitro stimulation of the cells, the peptide of SEQ ID NO: 4 is dissolved in DMSO at a concentration of 40 mg/ml, diluted with RPMI 1640 with 10% FBS to 40 μg/ml, and added to the splenocyte-containing wells at a final concentration of 10 μg/ml. The plate is incubated for about 17 hours at 37° C. under an atmosphere of 5% CO₂. Then, after removal of the culture medium from the wells, the ELISPOT plate is subjected to treatment for cell staining in accordance with the manufacturer's protocol. Stained spots are counted on ImmunoSpot Analyzer (C.T.L.).

### INDUSTRIAL APPLICABILITY

The compound of the disclosure efficiently induces CTLs, has favorable physicochemical stability, enables easy manufacturing and simple manufacturing control, and can be used widely. Also, the cocktail vaccine of the disclosure comprising the compound of the disclosure and an MHC class I-restricted peptide is useful as it induces CTLs more efficiently.

1. A compound of formula (1): 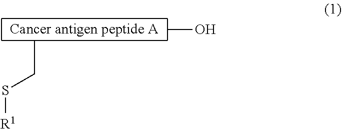
wherein cancer antigen peptide A is an MHC class I-restricted WT1 peptide consisting of 7 to 30 amino acid residues and containing at least one cysteine residue, wherein the cysteine residue of the cancer antigen peptide A binds to R¹ via a disulfide bond; and
R¹ is a group of formula (2), the group of formula (3), or cancer antigen peptide D,
wherein the group of formula (2) is 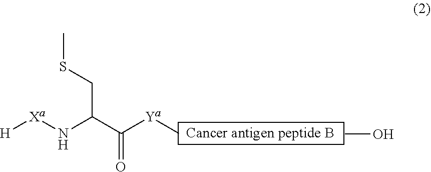
wherein X^{a} and Y^{a} independently represent a single bond or a divalent peptide group consisting of 1 to 4 amino acid residues, provided that the sum of the number of amino acid residues in X^{a} and Y^{a} is an integer of 0 to 4, and
the cancer antigen peptide B is an MHC class II-restricted WT1 peptide consisting of 9 to 30 amino acid residues, wherein the amino group of the N-terminal amino acid of the cancer antigen peptide B binds to Y^{a} in the formula (2), and the carbonyl group of the C-terminal amino acid of the cancer antigen peptide B binds to the hydroxyl group in the formula (2), and
the formula (1) and the formula (2) binds via a disulfide bond,
the group of formula (3) is 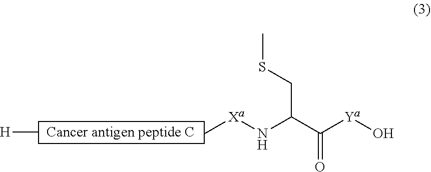
wherein X^{b} and Y^{b} independently represent a single bond or a divalent peptide group consisting of 1 to 4 amino acid residues, provided that the sum of the number of amino acid residues in X^{b} and Y^{b} is an integer of 0 to 4, and
the cancer antigen peptide C is an MHC class II-restricted WT1 peptide consisting of 9 to 30 amino acid residues, wherein the carbonyl group of the C-terminal amino acid of the cancer antigen peptide C binds to X^{b} in the formula (3), and the amino group of the N-terminal amino acid of the cancer antigen peptide C binds to the hydrogen atom in the formula (3), and the formula (1) and the formula (3) binds via a disulfide bond, and
the cancer antigen peptide D is an MHC class II-restricted WT1 peptide consisting of 9 to 30 amino acid residues and containing at least one cysteine, wherein the cysteine residue of the cancer antigen peptide D binds to R¹ via a disulfide bond;
or a pharmaceutically acceptable salt thereof.

2. The compound or pharmaceutically acceptable salt thereof of claim 1, wherein the cancer antigen peptide A is an MHC class I-restricted WT peptide consisting of 7 to 12 amino acid residues.

3. The compound or pharmaceutically acceptable salt thereof of claim 1, wherein the cancer antigen peptide A is a peptide comprising the amino acid sequence:
(SEQ ID NO: 3)
CMTWNQMNL
or a peptide comprising an amino acid sequence that differs from the amino acid sequence of SEQ ID NO: 3 by deletion, substitution, or addition of one to three amino acid residues and having an ability to induce CTLs.

4. The compound or pharmaceutically acceptable salt thereof of claim 3, wherein the cancer antigen peptide A is a peptide comprising an amino acid sequence selected from the group consisting of
(SEQ ID NO: 3)
CMTWNQMNL
and
(SEQ ID NO: 4)
CYTWNQMNL.

5. The compound or pharmaceutically acceptable salt thereof of claim 1, wherein R¹ is the group of formula (2).

6. The compound or pharmaceutically acceptable salt thereof of claim 5, wherein X^{a} and Y^{a} are single bonds.

7. The compound or pharmaceutically acceptable salt thereof of claim, wherein the cancer antigen peptide B is an MHC class II-restricted WT1 peptide consisting of 10 to 25 amino acid residues.

8. The compound or pharmaceutically acceptable salt thereof of claim 5, wherein the cancer antigen peptide B is a peptide consisting of an amino acid sequence selected from the group consisting of:
(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
and
(SEQ ID NO: 248)
RYFKLSHLQMHSRKHTGE
or a peptide consisting of an amino acid sequence that differs from the amino acid sequence selected from the group consisting of SEQ ID NOS: 217-248 by deletion, substitution, or addition of one to three amino acid residues and having an ability to induce helper T cells.

9. The compound or pharmaceutically acceptable salt thereof of claim 1, wherein R¹ is the group of formula (3).

10. The compound or pharmaceutically acceptable salt thereof of claim 9, wherein X^{b} and Y^{b} are single bonds.

11. The compound or pharmaceutically acceptable salt thereof of claim 9, wherein the cancer antigen peptide C is an MHC class II-restricted WT peptide consisting of 10 to 25 amino acid residues.

12. The compound or pharmaceutically acceptable salt thereof of claim 9, wherein the cancer antigen peptide C is a peptide consisting of an amino acid sequence selected from the group consisting of:
(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
and
(SEQ ID NO: 248)
RYFKLSHLQMHSRKHTGE,
or a peptide consisting of an amino acid sequence that differs from the amino acid sequence selected from the group consisting of SEQ ID NOS: 217-248 by deletion, substitution, or addition of one to three amino acid residues and having an ability to induce helper T cells.

13. The compound or pharmaceutically acceptable salt thereof of claim 1, wherein R¹ is the cancer antigen peptide D.

14. The compound or pharmaceutically acceptable salt thereof of claim 13, wherein the cancer antigen peptide D is an MHC class II-restricted WT peptide consisting of 10 to 25 amino acid residues.

15. The compound or pharmaceutically acceptable salt thereof of claim 13, wherein the cancer antigen peptide D is a peptide consisting of an amino acid sequence containing at least one cysteine residue selected from the group consisting of:
(SEQ ID NO: 217)
SGQARMFPNAPYLPSC,
(SEQ ID NO: 218)
SGQAYMFPNAPYLPSC,
(SEQ ID NO: 219)
SGQARMFPNAPYLPSCLES,
(SEQ ID NO: 220)
SGQAYMFPNAPYLPSCLES,
(SEQ ID NO: 221)
AYPGCNKRYFKLSHL,
(SEQ ID NO: 222)
YPGCNKRYFKLSHLQ,
(SEQ ID NO: 223)
KRYFKLSHLQMHSRK,
(SEQ ID NO: 224)
RYFKLSHLQMHSRKH,
(SEQ ID NO: 225)
YFKLSHLQMHSRKHT,
(SEQ ID NO: 226)
FKLSHLQMHSRKHTG,
(SEQ ID NO: 227)
KLSHLQMHSRKHTGE,
(SEQ ID NO: 228)
NKRYFKLSHLQMHSRK,
(SEQ ID NO: 229)
KRYFKLSHLQMHSRKH,
(SEQ ID NO: 230)
GCNKRYFKLSHLQMHSRK,
(SEQ ID NO: 231)
PGCNKRYFKLSHLQMHSRK,
(SEQ ID NO: 232)
PGCNKRYFKLSHLQMHSRKH,
(SEQ ID NO: 233)
PGCNKRYFKLSHLQMHSRKHTG,
(SEQ ID NO: 234)
CNKRYFKLSHLQMHSRK,
(SEQ ID NO: 235)
CNKRYFKLSHLQMHSRKH,
(SEQ ID NO: 236)
CNKRYFKLSHLQMHSRKHTG,
(SEQ ID NO: 237)
WAPVLDFAPPGASAYGSL,
(SEQ ID NO: 238)
CWAPVLDFAPPGASAYGSL,
(SEQ ID NO: 239)
WAPVLDFAPPGASAYGSLC,
(SEQ ID NO: 240)
EQCLSAFTLHFSGQFTG,
(SEQ ID NO: 241)
FRGIQDVRRVSGVAPTLVR,
(SEQ ID NO: 242)
RYFKLSHLQMHSRK,
(SEQ ID NO: 243)
AYPGCNKRYFKLSHLQMH,
(SEQ ID NO: 244)
AYPGCNKRYFKLSHLQMHSRK,
(SEQ ID NO: 245)
RYFKLSHLQMH,
(SEQ ID NO: 246)
GCNKRYFKLSHL,
(SEQ ID NO: 247)
RYFKLSHLQMHSRKHT,
and
(SEQ ID NO: 248)
RYFKLSHLQMHSRKHTGE,
or a peptide consisting of an amino acid sequence that differs from the amino acid sequence selected from the group consisting of SEQ ID NOS: 217-248 by deletion, substitution, or addition of one to three amino acid residues and contains at least one cysteine residue and having an ability to induce helper T cells.

16. The compound or pharmaceutically acceptable salt thereof of claim 1, wherein the compound of formula (1) is a compound of formula (4): 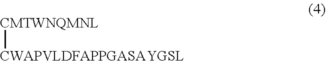
wherein C—C shown in the formula means that the C residues are linked together by a disulfide bond.

17. The compound or pharmaceutically acceptable salt thereof of claim 1, wherein the compound of formula (1) is a compound of formula (5): 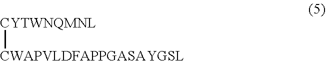
wherein C—C shown in the formula means that the C residues are linked together by a disulfide bond.

18. The compound or pharmaceutically acceptable salt thereof of claim 1, wherein the compound of formula (1) is a compound of formula (6): 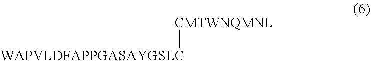
wherein C—C shown in the formula means that the C residues are linked together by a disulfide bond.

19. The compound or pharmaceutically acceptable salt thereof of claim 1, wherein the compound of formula (1) is a compound of formula (7): 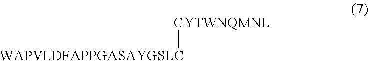
wherein C—C shown in the formula means that the C residues are linked together by a disulfide bond.

20. A composition comprising the compound or pharmaceutically acceptable salt thereof of claim 1 and at least one cancer antigen peptide E or a pharmaceutically acceptable salt thereof, wherein the at least one cancer antigen peptide E is an MHC class I-restricted WT1 peptide consisting of 7 to 30 amino acid residues.

21. The composition of claim 20, wherein the at least one cancer antigen peptide E is an MHC class I-restricted WT1 peptide consisting of 7 to 12 amino acid residues.

22. The composition of claim 20, wherein the at least one cancer antigen peptide E is a peptide different from the cancer antigen peptide A.

23. The composition of claim 20, wherein the at least one cancer antigen peptide E includes a peptide consisting of an amino acid sequence selected from the group consisting of:
(SEQ ID NO: 2)
RMFPNAPYL,
(SEQ ID NO: 209)
YMFPNAPYL,
(SEQ ID NO: 3)
CMTWNQMNL,
(SEQ ID NO: 4)
CYTWNQMNL,
(SEQ ID NO: 5)
ALLPAVPSL,
(SEQ ID NO: 6)
SLGEQQYSV,
and
(SEQ ID NO: 7)
RVPGVAPTL
or a peptide consisting of an amino acid sequence that differs from the amino acid sequence selected from the group consisting of SEQ ID NOS: 2, 3, 5, 6 and 7 by deletion, substitution, or addition of one to three amino acid residues and having an ability to induce CTLs.

24. A composition comprising at least one compound selected from the group consisting of a compound of formula (4): 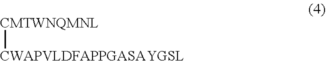
wherein C—C shown in the formula means that the C residues are linked together by a disulfide bond,
a compound of formula (5): 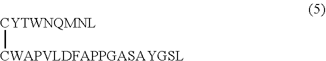
wherein C—C shown in the formula means that the C residues are linked together by a disulfide bond,
a compound of formula (6): 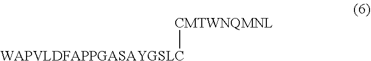
wherein C—C shown in the formula means that the C residues are linked together by a disulfide bond, and
a compound of formula (7): 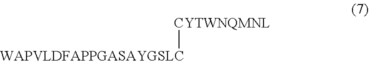
wherein C—C shown in the formula means that the C residues are linked together by a disulfide bond,
or a pharmaceutically acceptable salt thereof, and
at least one peptide consisting an amino acid sequence selected from the group consisting of:
(SEQ ID NO: 2)
RMFPNAPYL,
(SEQ ID NO: 209)
YMFPNAPYL,
(SEQ ID NO: 3)
CMTWNQMNL,
(SEQ ID NO: 4)
CYTWNQMNL,
(SEQ ID NO: 5)
ALLPAVPSL,
(SEQ ID NO: 6)
SLGEQQYSV,
and
(SEQ ID NO: 7)
RVPGVAPTL
or a pharmaceutically acceptable salt thereof.

25. A pharmaceutical composition comprising the compound or pharmaceutically acceptable salt thereof of claim 1, and a pharmaceutically acceptable carrier.

26. The pharmaceutical composition of claim 25, wherein the pharmaceutical composition is for use as a composition for treating a cancer associated with WT1 gene expression or an elevated level of WT gene expression.

27. The pharmaceutical composition of claim 25, wherein the pharmaceutical composition is for use as a composition for inducing CTLs in cellular immunotherapy for a cancer.

28. The pharmaceutical composition of claim 25, wherein the pharmaceutical composition is for use as a cancer vaccine.

29. The pharmaceutical composition of claim 26, wherein the cancer is a hematologic cancer selected from the group consisting of leukemia, myelodysplastic syndrome, multiple myeloma, and malignant lymphoma; or a solid cancer selected from the group consisting of gastric cancer, colorectal cancer, lung cancer, breast cancer, germ cell cancer, liver cancer, skin cancer, urinary bladder cancer, prostate cancer, uterine cancer, cervical cancer, ovarian cancer, and brain tumor.

30. A method of treating or preventing a cancer, comprising administering to a WT1-positive subject in need thereof a therapeutically or prophylactically effective amount of the compound or pharmaceutically acceptable salt thereof of claim 1.


SEQUENCE LISTING (272 sequences)

SEQ ID NO: 1 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 2 - protein, 9 aa, Artificial Sequence
  Peptide
RMFPNAPYL

SEQ ID NO: 3 - protein, 9 aa, Artificial Sequence
  Peptide
CMTWNQMNL

SEQ ID NO: 4 - protein, 9 aa, Artificial Sequence
  Peptide
CYTWNQMNL

SEQ ID NO: 5 - protein, 9 aa, Artificial Sequence
  Peptide
ALLPAVPSL

SEQ ID NO: 6 - protein, 9 aa, Artificial Sequence
  Peptide
SLGEQQYSV

SEQ ID NO: 7 - protein, 9 aa, Artificial Sequence
  Peptide
RVPGVAPTL

SEQ ID NO: 8 - protein, 9 aa, Artificial Sequence
  Peptide
GSDVRDLNA

SEQ ID NO: 9 - protein, 9 aa, Artificial Sequence
  Peptide
SDVRDLNAL

SEQ ID NO: 10 - protein, 9 aa, Artificial Sequence
  Peptide
DVRDLNALL

SEQ ID NO: 11 - protein, 9 aa, Artificial Sequence
  Peptide
RDLNALLPA

SEQ ID NO: 12 - protein, 9 aa, Artificial Sequence
  Peptide
DLNALLPAV

SEQ ID NO: 13 - protein, 9 aa, Artificial Sequence
  Peptide
SLGGGGGCA

SEQ ID NO: 14 - protein, 9 aa, Artificial Sequence
  Peptide
LGGGGGCAL

SEQ ID NO: 15 - protein, 9 aa, Artificial Sequence
  Peptide
GGGGCALPV

SEQ ID NO: 16 - protein, 9 aa, Artificial Sequence
  Peptide
GCALPVSGA

SEQ ID NO: 17 - protein, 9 aa, Artificial Sequence
  Peptide
CALPVSGAA

SEQ ID NO: 18 - protein, 9 aa, Artificial Sequence
  Peptide
LPVSGAAQW

SEQ ID NO: 19 - protein, 9 aa, Artificial Sequence
  Peptide
SGAAQWAPV

SEQ ID NO: 20 - protein, 9 aa, Artificial Sequence
  Peptide
GAAQWAPVL

SEQ ID NO: 21 - protein, 9 aa, Artificial Sequence
  Peptide
AQWAPVLDF

SEQ ID NO: 22 - protein, 9 aa, Artificial Sequence
  Peptide
QWAPVLDFA

SEQ ID NO: 23 - protein, 9 aa, Artificial Sequence
  Peptide
VLDFAPPGA

SEQ ID NO: 24 - protein, 9 aa, Artificial Sequence
  Peptide
LDFAPPGAS

SEQ ID NO: 25 - protein, 9 aa, Artificial Sequence
  Peptide
DFAPPGASA

SEQ ID NO: 26 - protein, 9 aa, Artificial Sequence
  Peptide
FAPPGASAY

SEQ ID NO: 27 - protein, 9 aa, Artificial Sequence
  Peptide
AYGSLGGPA

SEQ ID NO: 28 - protein, 9 aa, Artificial Sequence
  Peptide
PPPPPPHSF

SEQ ID NO: 29 - protein, 9 aa, Artificial Sequence
  Peptide
PPPPPHSFI

SEQ ID NO: 30 - protein, 9 aa, Artificial Sequence
  Peptide
PPPPHSFIK

SEQ ID NO: 31 - protein, 9 aa, Artificial Sequence
  Peptide
SFIKQEPSW

SEQ ID NO: 32 - protein, 9 aa, Artificial Sequence
  Peptide
KQEPSWGGA

SEQ ID NO: 33 - protein, 9 aa, Artificial Sequence
  Peptide
GAEPHEEQC

SEQ ID NO: 34 - protein, 9 aa, Artificial Sequence
  Peptide
AEPHEEQCL

SEQ ID NO: 35 - protein, 9 aa, Artificial Sequence
  Peptide
EPHEEQCLS

SEQ ID NO: 36 - protein, 9 aa, Artificial Sequence
  Peptide
PHEEQCLSA

SEQ ID NO: 37 - protein, 9 aa, Artificial Sequence
  Peptide
HEEQCLSAF

SEQ ID NO: 38 - protein, 9 aa, Artificial Sequence
  Peptide
EEQCLSAFT

SEQ ID NO: 39 - protein, 9 aa, Artificial Sequence
  Peptide
EQCLSAFTV

SEQ ID NO: 40 - protein, 9 aa, Artificial Sequence
  Peptide
CLSAFTVHF

SEQ ID NO: 41 - protein, 9 aa, Artificial Sequence
  Peptide
FTVHFSGQF

SEQ ID NO: 42 - protein, 9 aa, Artificial Sequence
  Peptide
TVHFSGQFT

SEQ ID NO: 43 - protein, 9 aa, Artificial Sequence
  Peptide
FSGQFTGTA

SEQ ID NO: 44 - protein, 9 aa, Artificial Sequence
  Peptide
GQFTGTAGA

SEQ ID NO: 45 - protein, 9 aa, Artificial Sequence
  Peptide
QFTGTAGAC

SEQ ID NO: 46 - protein, 9 aa, Artificial Sequence
  Peptide
FTGTAGACR

SEQ ID NO: 47 - protein, 9 aa, Artificial Sequence
  Peptide
TGTAGACRY

SEQ ID NO: 48 - protein, 9 aa, Artificial Sequence
  Peptide
AGACRYGPF

SEQ ID NO: 49 - protein, 9 aa, Artificial Sequence
  Peptide
CRYGPFGPP

SEQ ID NO: 50 - protein, 9 aa, Artificial Sequence
  Peptide
GPFGPPPPS

SEQ ID NO: 51 - protein, 9 aa, Artificial Sequence
  Peptide
SQASSGQAR

SEQ ID NO: 52 - protein, 9 aa, Artificial Sequence
  Peptide
QASSGQARM

SEQ ID NO: 53 - protein, 9 aa, Artificial Sequence
  Peptide
ASSGQARMF

SEQ ID NO: 54 - protein, 9 aa, Artificial Sequence
  Peptide
GQARMFPNA

SEQ ID NO: 55 - protein, 9 aa, Artificial Sequence
  Peptide
ARMFPNAPY

SEQ ID NO: 56 - protein, 9 aa, Artificial Sequence
  Peptide
FPNAPYLPS

SEQ ID NO: 57 - protein, 9 aa, Artificial Sequence
  Peptide
NAPYLPSCL

SEQ ID NO: 58 - protein, 9 aa, Artificial Sequence
  Peptide
SCLESQPAI

SEQ ID NO: 59 - protein, 9 aa, Artificial Sequence
  Peptide
CLESQPAIR

SEQ ID NO: 60 - protein, 9 aa, Artificial Sequence
  Peptide
LESQPAIRN

SEQ ID NO: 61 - protein, 9 aa, Artificial Sequence
  Peptide
ESQPAIRNQ

SEQ ID NO: 62 - protein, 9 aa, Artificial Sequence
  Peptide
QPAIRNQGY

SEQ ID NO: 63 - protein, 9 aa, Artificial Sequence
  Peptide
AIRNQGYST

SEQ ID NO: 64 - protein, 9 aa, Artificial Sequence
  Peptide
IRNQGYSTV

SEQ ID NO: 65 - protein, 9 aa, Artificial Sequence
  Peptide
NQGYSTVTF

SEQ ID NO: 66 - protein, 9 aa, Artificial Sequence
  Peptide
VTFDGTPSY

SEQ ID NO: 67 - protein, 9 aa, Artificial Sequence
  Peptide
GHTPSHHAA

SEQ ID NO: 68 - protein, 9 aa, Artificial Sequence
  Peptide
TPSHHAAQF

SEQ ID NO: 69 - protein, 9 aa, Artificial Sequence
  Peptide
SHHAAQFPN

SEQ ID NO: 70 - protein, 9 aa, Artificial Sequence
  Peptide
AAQFPNHSF

SEQ ID NO: 71 - protein, 9 aa, Artificial Sequence
  Peptide
AQFPNHSFK

SEQ ID NO: 72 - protein, 9 aa, Artificial Sequence
  Peptide
HSFKHEDPM

SEQ ID NO: 73 - protein, 9 aa, Artificial Sequence
  Peptide
KHEDPMGQQ

SEQ ID NO: 74 - protein, 9 aa, Artificial Sequence
  Peptide
EDPMGQQGS

SEQ ID NO: 75 - protein, 9 aa, Artificial Sequence
  Peptide
DPMGQQGSL

SEQ ID NO: 76 - protein, 9 aa, Artificial Sequence
  Peptide
QGSLGEQQY

SEQ ID NO: 77 - protein, 9 aa, Artificial Sequence
  Peptide
QQYSVPPPV

SEQ ID NO: 78 - protein, 9 aa, Artificial Sequence
  Peptide
QYSVPPPVY

SEQ ID NO: 79 - protein, 9 aa, Artificial Sequence
  Peptide
SVPPPVYGC

SEQ ID NO: 80 - protein, 9 aa, Artificial Sequence
  Peptide
CHTPTDSCT

SEQ ID NO: 81 - protein, 9 aa, Artificial Sequence
  Peptide
TPTDSCTGS

SEQ ID NO: 82 - protein, 9 aa, Artificial Sequence
  Peptide
TDSCTGSQA

SEQ ID NO: 83 - protein, 9 aa, Artificial Sequence
  Peptide
DSCTGSQAL

SEQ ID NO: 84 - protein, 9 aa, Artificial Sequence
  Peptide
SCTGSQALL

SEQ ID NO: 85 - protein, 9 aa, Artificial Sequence
  Peptide
CTGSQALLL

SEQ ID NO: 86 - protein, 9 aa, Artificial Sequence
  Peptide
TGSQALLLR

SEQ ID NO: 87 - protein, 9 aa, Artificial Sequence
  Peptide
GSQALLLRT

SEQ ID NO: 88 - protein, 9 aa, Artificial Sequence
  Peptide
QALLLRTPY

SEQ ID NO: 89 - protein, 9 aa, Artificial Sequence
  Peptide
LRTPYSSDN

SEQ ID NO: 90 - protein, 9 aa, Artificial Sequence
  Peptide
RTPYSSDNL

SEQ ID NO: 91 - protein, 9 aa, Artificial Sequence
  Peptide
TPYSSDNLY

SEQ ID NO: 92 - protein, 9 aa, Artificial Sequence
  Peptide
YSSDNLYQM

SEQ ID NO: 93 - protein, 9 aa, Artificial Sequence
  Peptide
SSDNLYQMT

SEQ ID NO: 94 - protein, 9 aa, Artificial Sequence
  Peptide
SDNLYQMTS

SEQ ID NO: 95 - protein, 9 aa, Artificial Sequence
  Peptide
NLYQMTSQL

SEQ ID NO: 96 - protein, 9 aa, Artificial Sequence
  Peptide
YQMTSQLEC

SEQ ID NO: 97 - protein, 9 aa, Artificial Sequence
  Peptide
QMTSQLECM

SEQ ID NO: 98 - protein, 9 aa, Artificial Sequence
  Peptide
TSQLECMTW

SEQ ID NO: 99 - protein, 9 aa, Artificial Sequence
  Peptide
QLECMTWNQ

SEQ ID NO: 100 - protein, 9 aa, Artificial Sequence
  Peptide
LECMTWNQM

SEQ ID NO: 101 - protein, 9 aa, Artificial Sequence
  Peptide
NQMNLGATL

SEQ ID NO: 102 - protein, 9 aa, Artificial Sequence
  Peptide
QMNLGATLK

SEQ ID NO: 103 - protein, 9 aa, Artificial Sequence
  Peptide
NLGATLKGV

SEQ ID NO: 104 - protein, 9 aa, Artificial Sequence
  Peptide
LGATLKGVA

SEQ ID NO: 105 - protein, 9 aa, Artificial Sequence
  Peptide
GATLKGVAA

SEQ ID NO: 106 - protein, 9 aa, Artificial Sequence
  Peptide
VAAGSSSSV

SEQ ID NO: 107 - protein, 9 aa, Artificial Sequence
  Peptide
AAGSSSSVK

SEQ ID NO: 108 - protein, 9 aa, Artificial Sequence
  Peptide
AGSSSSVKW

SEQ ID NO: 109 - protein, 9 aa, Artificial Sequence
  Peptide
WTEGQSNHS

SEQ ID NO: 110 - protein, 9 aa, Artificial Sequence
  Peptide
TEGQSNHST

SEQ ID NO: 111 - protein, 9 aa, Artificial Sequence
  Peptide
GQSNHSTGY

SEQ ID NO: 112 - protein, 9 aa, Artificial Sequence
  Peptide
TGYESDNHT

SEQ ID NO: 113 - protein, 9 aa, Artificial Sequence
  Peptide
GYESDNHTT

SEQ ID NO: 114 - protein, 9 aa, Artificial Sequence
  Peptide
ESDNHTTPI

SEQ ID NO: 115 - protein, 9 aa, Artificial Sequence
  Peptide
SDNHTTPIL

SEQ ID NO: 116 - protein, 9 aa, Artificial Sequence
  Peptide
HTTPILCGA

SEQ ID NO: 117 - protein, 9 aa, Artificial Sequence
  Peptide
TPILCGAQY

SEQ ID NO: 118 - protein, 9 aa, Artificial Sequence
  Peptide
PILCGAQYR

SEQ ID NO: 119 - protein, 9 aa, Artificial Sequence
  Peptide
ILCGAQYRI

SEQ ID NO: 120 - protein, 9 aa, Artificial Sequence
  Peptide
QYRIHTHGV

SEQ ID NO: 121 - protein, 9 aa, Artificial Sequence
  Peptide
YRIHTHGVF

SEQ ID NO: 122 - protein, 9 aa, Artificial Sequence
  Peptide
RIHTHGVFR

SEQ ID NO: 123 - protein, 9 aa, Artificial Sequence
  Peptide
HTHGVFRGI

SEQ ID NO: 124 - protein, 9 aa, Artificial Sequence
  Peptide
GVFRGIQDV

SEQ ID NO: 125 - protein, 9 aa, Artificial Sequence
  Peptide
VFRGIQDVR

SEQ ID NO: 126 - protein, 9 aa, Artificial Sequence
  Peptide
FRGIQDVRR

SEQ ID NO: 127 - protein, 9 aa, Artificial Sequence
  Peptide
RGIQDVRRV

SEQ ID NO: 128 - protein, 9 aa, Artificial Sequence
  Peptide
QDVRRVPGV

SEQ ID NO: 129 - protein, 9 aa, Artificial Sequence
  Peptide
DVRRVPGVA

SEQ ID NO: 130 - protein, 9 aa, Artificial Sequence
  Peptide
RRVPGVAPT

SEQ ID NO: 131 - protein, 9 aa, Artificial Sequence
  Peptide
VPGVAPTLV

SEQ ID NO: 132 - protein, 9 aa, Artificial Sequence
  Peptide
VAPTLVRSA

SEQ ID NO: 133 - protein, 9 aa, Artificial Sequence
  Peptide
TLVRSASET

SEQ ID NO: 134 - protein, 9 aa, Artificial Sequence
  Peptide
RSASETSEK

SEQ ID NO: 135 - protein, 9 aa, Artificial Sequence
  Peptide
SASETSEKR

SEQ ID NO: 136 - protein, 9 aa, Artificial Sequence
  Peptide
SETSEKRPF

SEQ ID NO: 137 - protein, 9 aa, Artificial Sequence
  Peptide
ETSEKRPFM

SEQ ID NO: 138 - protein, 9 aa, Artificial Sequence
  Peptide
TSEKRPFMC

SEQ ID NO: 139 - protein, 9 aa, Artificial Sequence
  Peptide
SEKRPFMCA

SEQ ID NO: 140 - protein, 9 aa, Artificial Sequence
  Peptide
EKRPFMCAY

SEQ ID NO: 141 - protein, 9 aa, Artificial Sequence
  Peptide
MCAYPGCNK

SEQ ID NO: 142 - protein, 9 aa, Artificial Sequence
  Peptide
CAYPGCNKR

SEQ ID NO: 143 - protein, 9 aa, Artificial Sequence
  Peptide
AYPGCNKRY

SEQ ID NO: 144 - protein, 9 aa, Artificial Sequence
  Peptide
YPGCNKRYF

SEQ ID NO: 145 - protein, 9 aa, Artificial Sequence
  Peptide
GCNKRYFKL

SEQ ID NO: 146 - protein, 9 aa, Artificial Sequence
  Peptide
KRYFKLSHL

SEQ ID NO: 147 - protein, 9 aa, Artificial Sequence
  Peptide
YFKLSHLQM

SEQ ID NO: 148 - protein, 9 aa, Artificial Sequence
  Peptide
LSHLQMHSR

SEQ ID NO: 149 - protein, 9 aa, Artificial Sequence
  Peptide
LQMHSRKHT

SEQ ID NO: 150 - protein, 9 aa, Artificial Sequence
  Peptide
HSRKHTGEK

SEQ ID NO: 151 - protein, 9 aa, Artificial Sequence
  Peptide
RKHTGEKPY

SEQ ID NO: 152 - protein, 9 aa, Artificial Sequence
  Peptide
HTGEKPYQC

SEQ ID NO: 153 - protein, 9 aa, Artificial Sequence
  Peptide
GEKPYQCDF

SEQ ID NO: 154 - protein, 9 aa, Artificial Sequence
  Peptide
KPYQCDFKD

SEQ ID NO: 155 - protein, 9 aa, Artificial Sequence
  Peptide
QCDFKDCER

SEQ ID NO: 156 - protein, 9 aa, Artificial Sequence
  Peptide
DFKDCERRF

SEQ ID NO: 157 - protein, 9 aa, Artificial Sequence
  Peptide
KDCERRFSR

SEQ ID NO: 158 - protein, 9 aa, Artificial Sequence
  Peptide
RRFSRSDQL

SEQ ID NO: 159 - protein, 9 aa, Artificial Sequence
  Peptide
RFSRSDQLK

SEQ ID NO: 160 - protein, 9 aa, Artificial Sequence
  Peptide
FSRSDQLKR

SEQ ID NO: 161 - protein, 9 aa, Artificial Sequence
  Peptide
RSDQLKRHQ

SEQ ID NO: 162 - protein, 9 aa, Artificial Sequence
  Peptide
DQLKRHQRR

SEQ ID NO: 163 - protein, 9 aa, Artificial Sequence
  Peptide
KRHQRRHTG

SEQ ID NO: 164 - protein, 9 aa, Artificial Sequence
  Peptide
RHQRRHTGV

SEQ ID NO: 165 - protein, 9 aa, Artificial Sequence
  Peptide
HQRRHTGVK

SEQ ID NO: 166 - protein, 9 aa, Artificial Sequence
  Peptide
RRHTGVKPF

SEQ ID NO: 167 - protein, 9 aa, Artificial Sequence
  Peptide
GVKPFQCKT

SEQ ID NO: 168 - protein, 9 aa, Artificial Sequence
  Peptide
FQCKTCQRK

SEQ ID NO: 169 - protein, 9 aa, Artificial Sequence
  Peptide
QCKTCQRKF

SEQ ID NO: 170 - protein, 9 aa, Artificial Sequence
  Peptide
KTCQRKFSR

SEQ ID NO: 171 - protein, 9 aa, Artificial Sequence
  Peptide
TCQRKFSRS

SEQ ID NO: 172 - protein, 9 aa, Artificial Sequence
  Peptide
QRKFSRSDH

SEQ ID NO: 173 - protein, 9 aa, Artificial Sequence
  Peptide
RKFSRSDHL

SEQ ID NO: 174 - protein, 9 aa, Artificial Sequence
  Peptide
KFSRSDHLK

SEQ ID NO: 175 - protein, 9 aa, Artificial Sequence
  Peptide
RSDHLKTHT

SEQ ID NO: 176 - protein, 9 aa, Artificial Sequence
  Peptide
DHLKTHTRT

SEQ ID NO: 177 - protein, 9 aa, Artificial Sequence
  Peptide
HTRTHTGKT

SEQ ID NO: 178 - protein, 9 aa, Artificial Sequence
  Peptide
TGKTSEKPF

SEQ ID NO: 179 - protein, 9 aa, Artificial Sequence
  Peptide
KTSEKPFSC

SEQ ID NO: 180 - protein, 9 aa, Artificial Sequence
  Peptide
TSEKPFSCR

SEQ ID NO: 181 - protein, 9 aa, Artificial Sequence
  Peptide
SEKPFSCRW

SEQ ID NO: 182 - protein, 9 aa, Artificial Sequence
  Peptide
KPFSCRWPS

SEQ ID NO: 183 - protein, 9 aa, Artificial Sequence
  Peptide
SCRWPSCQK

SEQ ID NO: 184 - protein, 9 aa, Artificial Sequence
  Peptide
CRWPSCQKK

SEQ ID NO: 185 - protein, 9 aa, Artificial Sequence
  Peptide
RWPSCQKKF

SEQ ID NO: 186 - protein, 9 aa, Artificial Sequence
  Peptide
WPSCQKKFA

SEQ ID NO: 187 - protein, 9 aa, Artificial Sequence
  Peptide
PSCQKKFAR

SEQ ID NO: 188 - protein, 9 aa, Artificial Sequence
  Peptide
SCQKKFARS

SEQ ID NO: 189 - protein, 9 aa, Artificial Sequence
  Peptide
KKFARSDEL

SEQ ID NO: 190 - protein, 9 aa, Artificial Sequence
  Peptide
KFARSDELV

SEQ ID NO: 191 - protein, 9 aa, Artificial Sequence
  Peptide
FARSDELVR

SEQ ID NO: 192 - protein, 9 aa, Artificial Sequence
  Peptide
ARSDELVRH

SEQ ID NO: 193 - protein, 9 aa, Artificial Sequence
  Peptide
RSDELVRHH

SEQ ID NO: 194 - protein, 9 aa, Artificial Sequence
  Peptide
SDELVRHHN

SEQ ID NO: 195 - protein, 9 aa, Artificial Sequence
  Peptide
DELVRHHNM

SEQ ID NO: 196 - protein, 9 aa, Artificial Sequence
  Peptide
VRHHNMHQR

SEQ ID NO: 197 - protein, 9 aa, Artificial Sequence
  Peptide
RHHNMHQRN

SEQ ID NO: 198 - protein, 9 aa, Artificial Sequence
  Peptide
HHNMHQRNM

SEQ ID NO: 199 - protein, 9 aa, Artificial Sequence
  Peptide
NMHQRNMTK

SEQ ID NO: 200 - protein, 9 aa, Artificial Sequence
  Peptide
MHQRNMTKL

SEQ ID NO: 201 - protein, 9 aa, Artificial Sequence
  Peptide
QRNMTKLQL

SEQ ID NO: 202 - protein, 9 aa, Artificial Sequence
  Peptide
RNMTKLQLA

SEQ ID NO: 203 - protein, 9 aa, Artificial Sequence
  Peptide
NMTKLQLAL

SEQ ID NO: 204 - protein, 9 aa, Artificial Sequence
  Peptide
PYFPNAPYL

SEQ ID NO: 205 - protein, 9 aa, Artificial Sequence
  Peptide
FMFPNAPYL

SEQ ID NO: 206 - protein, 9 aa, Artificial Sequence
  Peptide
RLFPNAPYL

SEQ ID NO: 207 - protein, 9 aa, Artificial Sequence
  Peptide
RMMPNAPYL

SEQ ID NO: 208 - protein, 9 aa, Artificial Sequence
  Peptide
RMFPNAPYV

SEQ ID NO: 209 - protein, 9 aa, Artificial Sequence
  Peptide
YMFPNAPYL

SEQ ID NO: 210 - protein, 9 aa, Artificial Sequence
  Peptide
  MISC_FEATURE (1)..(1): Xaa is Ser or Ala
XMTWNQMNL

SEQ ID NO: 211 - protein, 9 aa, Artificial Sequence
  Peptide
  MISC_FEATURE (1)..(1): Xaa is Ser, Ala, Abu, Arg, Lys, Orn, Cit, Leu, Phe, or Asn
XYTWNQMNL

SEQ ID NO: 212 - protein, 9 aa, Artificial Sequence
  Peptide
AYLPAVPSL

SEQ ID NO: 213 - protein, 9 aa, Artificial Sequence
  Peptide
FLGFQQYSV

SEQ ID NO: 214 - protein, 9 aa, Artificial Sequence
  Peptide
SMGEQQYSV

SEQ ID NO: 215 - protein, 9 aa, Artificial Sequence
  Peptide
SLMEQQYSV

SEQ ID NO: 216 - protein, 9 aa, Artificial Sequence
  Peptide
RYPGVAPTL

SEQ ID NO: 217 - protein, 16 aa, Artificial Sequence
  Peptide
SGQARMFPNAPYLPSC

SEQ ID NO: 218 - protein, 16 aa, Artificial Sequence
  Peptide
SGQAYMFPNAPYLPSC

SEQ ID NO: 219 - protein, 19 aa, Artificial Sequence
  Peptide
SGQARMFPNAPYLPSCLES

SEQ ID NO: 220 - protein, 19 aa, Artificial Sequence
  Peptide
SGQAYMFPNAPYLPSCLES

SEQ ID NO: 221 - protein, 15 aa, Artificial Sequence
  Peptide
AYPGCNKRYFKLSHL

SEQ ID NO: 222 - protein, 15 aa, Artificial Sequence
  Peptide
YPGCNKRYFKLSHLQ

SEQ ID NO: 223 - protein, 15 aa, Artificial Sequence
  Peptide
KRYFKLSHLQMHSRK

SEQ ID NO: 224 - protein, 15 aa, Artificial Sequence
  Peptide
RYFKLSHLQMHSRKH

SEQ ID NO: 225 - protein, 15 aa, Artificial Sequence
  Peptide
YFKLSHLQMHSRKHT

SEQ ID NO: 226 - protein, 15 aa, Artificial Sequence
  Peptide
FKLSHLQMHSRKHTG

SEQ ID NO: 227 - protein, 15 aa, Artificial Sequence
  Peptide
KLSHLQMHSRKHTGE

SEQ ID NO: 228 - protein, 16 aa, Artificial Sequence
  Peptide
NKRYFKLSHLQMHSRK

SEQ ID NO: 229 - protein, 16 aa, Artificial Sequence
  Peptide
KRYFKLSHLQMHSRKH

SEQ ID NO: 230 - protein, 18 aa, Artificial Sequence
  Peptide
GCNKRYFKLSHLQMHSRK

SEQ ID NO: 231 - protein, 19 aa, Artificial Sequence
  Peptide
PGCNKRYFKLSHLQMHSRK

SEQ ID NO: 232 - protein, 20 aa, Artificial Sequence
  Peptide
PGCNKRYFKLSHLQMHSRKH

SEQ ID NO: 233 - protein, 22 aa, Artificial Sequence
  Peptide
PGCNKRYFKLSHLQMHSRKHTG

SEQ ID NO: 234 - protein, 17 aa, Artificial Sequence
  Peptide
CNKRYFKLSHLQMHSRK

SEQ ID NO: 235 - protein, 18 aa, Artificial Sequence
  Peptide
CNKRYFKLSHLQMHSRKH

SEQ ID NO: 236 - protein, 20 aa, Artificial Sequence
  Peptide
CNKRYFKLSHLQMHSRKHTG

SEQ ID NO: 237 - protein, 18 aa, Artificial Sequence
  Peptide
WAPVLDFAPPGASAYGSL

SEQ ID NO: 238 - protein, 19 aa, Artificial Sequence
  Peptide
CWAPVLDFAPPGASAYGSL

SEQ ID NO: 239 - protein, 19 aa, Artificial Sequence
  Peptide
WAPVLDFAPPGASAYGSLC

SEQ ID NO: 240 - protein, 17 aa, Artificial Sequence
  Peptide
EQCLSAFTLHFSGQFTG

SEQ ID NO: 241 - protein, 19 aa, Artificial Sequence
  Peptide
FRGIQDVRRVSGVAPTLVR

SEQ ID NO: 242 - protein, 14 aa, Artificial Sequence
  Peptide
RYFKLSHLQMHSRK

SEQ ID NO: 243 - protein, 18 aa, Artificial Sequence
  Peptide
AYPGCNKRYFKLSHLQMH

SEQ ID NO: 244 - protein, 21 aa, Artificial Sequence
  Peptide
AYPGCNKRYFKLSHLQMHSRK

SEQ ID NO: 245 - protein, 11 aa, Artificial Sequence
  Peptide
RYFKLSHLQMH

SEQ ID NO: 246 - protein, 12 aa, Artificial Sequence
  Peptide
GCNKRYFKLSHL

SEQ ID NO: 247 - protein, 16 aa, Artificial Sequence
  Peptide
RYFKLSHLQMHSRKHT

SEQ ID NO: 248 - protein, 18 aa, Artificial Sequence
  Peptide
RYFKLSHLQMHSRKHTGE

SEQ ID NO: 249 - protein, 13 aa, Artificial Sequence
  Peptide
YFKLSHLQMHSRK

SEQ ID NO: 250 - protein, 12 aa, Artificial Sequence
  Peptide
FKLSHLQMHSRK

SEQ ID NO: 251 - protein, 11 aa, Artificial Sequence
  Peptide
KLSHLQMHSRK

SEQ ID NO: 252 - protein, 16 aa, Artificial Sequence
  Peptide
FKLSHLQMHSRKHTGE

SEQ ID NO: 253 - protein, 12 aa, Artificial Sequence
  Peptide
KLSHLQMHSRKH

SEQ ID NO: 254 - protein, 20 aa, Artificial Sequence
  Peptide
YPGCNKRYFKLSHLQMHSRK

SEQ ID NO: 255 - protein, 20 aa, Artificial Sequence
  Peptide
AYPGCNKRYFKLSHLQMHSR

SEQ ID NO: 256 - protein, 19 aa, Artificial Sequence
  Peptide
AYPGCNKRYFKLSHLQMHS

SEQ ID NO: 257 - protein, 17 aa, Artificial Sequence
  Peptide
AYPGCNKRYFKLSHLQM

SEQ ID NO: 258 - protein, 16 aa, Artificial Sequence
  Peptide
AYPGCNKRYFKLSHLQ

SEQ ID NO: 259 - protein, 17 aa, Artificial Sequence
  Peptide
YFKLSHLQMHSRKHTGE

SEQ ID NO: 260 - protein, 17 aa, Artificial Sequence
  Peptide
RYFKLSHLQMHSRKHTG

SEQ ID NO: 261 - protein, 13 aa, Artificial Sequence
  Peptide
RYFKLSHLQMHSR

SEQ ID NO: 262 - protein, 12 aa, Artificial Sequence
  Peptide
RYFKLSHLQMHS

SEQ ID NO: 263 - protein, 10 aa, Artificial Sequence
  Peptide
RYFKLSHLQM

SEQ ID NO: 264 - protein, 14 aa, Artificial Sequence
  Peptide
YPGCNKRYFKLSHL

SEQ ID NO: 265 - protein, 13 aa, Artificial Sequence
  Peptide
PGCNKRYFKLSHL

SEQ ID NO: 266 - protein, 11 aa, Artificial Sequence
  Peptide
CNKRYFKLSHL

SEQ ID NO: 267 - protein, 10 aa, Artificial Sequence
  Peptide
NKRYFKLSHL

SEQ ID NO: 268 - protein, 14 aa, Artificial Sequence
  Peptide
KLSHLQMHSRKHTG

SEQ ID NO: 269 - protein, 13 aa, Artificial Sequence
  Peptide
KLSHLQMHSRKHT

SEQ ID NO: 270 - protein, 11 aa, Artificial Sequence
  Peptide
KLSHLQMHSRK

SEQ ID NO: 271 - protein, 10 aa, Artificial Sequence
  Peptide
KLSHLQMHSR

SEQ ID NO: 272 - protein, 55 aa, Artificial Sequence
  Amino acid sequence at positions 311-365 of SEQ ID NO 1
VRSASETSEKRPFMCAYPGCNKRYFKLSHLQMHSRKHTGEKPYQCDFKDCERRFS